(19) United States
(12) Patent Application Publication
(10) Pub. No.: US 2025/0061972 A1
(43) Pub. Date: Feb. 20, 2025

(54) MOLECULAR RESPONSE AND PROGRESSION DETECTION FROM CIRCULATING CELL FREE DNA

(71) Applicant: Tempus AI, Inc., Chicago, IL (US)

(72) Inventors: Justin David Finkle, Chicago, IL (US); Christine Y. Lee, Menlo Park, CA (US); Jonathan Alexander Heiss, Chicago, IL (US); Robert Tell, Chicago, IL (US); Sun Hae Hong, Belmont, CA (US)

(21) Appl. No.: 18/938,173

(22) Filed: Nov. 5, 2024

Related U.S. Application Data
(63) Continuation of application No. 17/859,599, filed on Jul. 7, 2022.
Application No. 17/859,599 is a continuation of application No. 17/352,231, filed on Jun. 18, 2021, now abandoned.
(60) Provisional application No. 63/041,692, filed on Jun. 19, 2020.

(51) Int. Cl. G16B 40/00; G06F 30/27; G06F 111/08; G16B 5/20; G16B 20/20; G16B 30/00; G16B 40/20

(52) U.S. Cl. CPC G16B 40/00; G06F 30/27; G16B 5/20; G16B 20/20; G16B 30/00; G16B 40/20; G06F 2111/08

(57) ABSTRACT

Methods, systems, and software are provided for monitoring a cancer condition of a test subject. The method includes obtaining a liquid biopsy sample from the subject at a second time point, occurring after a first time point, containing cell-free DNA fragments. Low-pass whole genome methylation sequencing of the cell-free DNA fragments is performed to obtain nucleic acid sequences having a methylation pattern for a corresponding cell-free DNA fragment. The nucleic acid sequences are mapped to a location on a reference genome. Methylation metrics are determined based on the methylation patterns and mapped locations of the nucleic acid sequences. A circulating tumor fraction is estimated from the methylation metrics, and the estimate is compared to an estimate of the circulating tumor fraction for the test subject at the first time point.

### CROSS REFERENCE TO RELATED APPLICATIONS

This application is a continuation of U.S. patent application Ser. No. 17/859,599, filed Jul. 7, 2022, which is a continuation of U.S. patent application Ser. No. 17/352,231, filed Jun. 18, 2021, which claims priority to U.S. Provisional Patent Application No. 63/041,692, filed Jun. 19, 2020, the contents of which are hereby incorporated by reference, in their entireties, for all purposes.

### FIELD OF THE INVENTION

The present disclosure relates generally to the use of whole genome methylation sequencing, e.g., low-pass whole genome methylation sequencing, of liquid biopsy samples for early cancer detection and circulating tumor fraction estimation.

### BACKGROUND

Multiple lines of evidence suggest that early cancer detection results in significantly improved treatment outcomes. However, because different methodologies are used to screen for different types of cancers, conventional cancer detection is expensive, inefficient, invasive, and time consuming. For instance, mammography is conventionally used to screen for breast cancer, colonoscopies are used to screen for colon cancer, and Pap tests are used to screen for cervical cancer. These procedures are uncomfortable and/or significantly invasive to the patient, are commonly performed in separate visits to a clinical environment, and even when combined screen for only three types of cancer. Perhaps most troubling, the standard of care for cancer screening varies significantly in quality, funding, and recommendation between different types of cancers without a clear clinical rationale. Corley D A et al., JAMA, 315 (19): 2067-68 (2016). For instance, while the CDC offers free or low-cost breast and cervical cancer screening programs, they do not recommend screening for many types of cancers, such as ovarian, pancreatic, prostate, testicular, thyroid, bladder, oral, or skin cancer, at all.

Cell-free DNA (cfDNA) has been identified in various bodily fluids, e.g., blood serum, plasma, urine, etc. Chan et al., Ann. Clin. Biochem., 40 (Pt 2): 122-30 (2003). This cfDNA originates from necrotic or apoptotic cells of all types, including germline cells, hematopoietic cells, and diseased (e.g., cancerous) cells. Advantageously, genomic alterations in cancerous tissues can be identified from cfDNA isolated from cancer patients. Sec, e.g., Stroun et al., Oncology, 46 (5): 318-22 (1989); Goessl et al., Cancer Res., 60 (21): 5941-45 (2000); and Frenel et al., Clin. Cancer Res. 21 (20): 4586-96 (2015). Thus, one approach to overcoming the problems with conventional cancer screening methodologies described above is to analyze cell-free nucleic acids (e.g., cfDNA) and/or nucleic acids in circulating tumor cells present in biological fluids, e.g., via a liquid biopsy. However, the implementation of liquid biopsy assays in clinical practice has been hindered by the expense of high-depth next generation sequencing deemed necessary for precision oncology and cancer screening methodologies. For instance, an economic evaluation found it was not cost effective to use liquid biopsy assays to aid in clinical decision making during treatment of Her2-positive advanced breast cancer. Sánchez-Calderón D. et al., ClinicoEconomic and Outcomes Research, 12:115-22 (2020).

Precision oncology is the practice of tailoring cancer therapy to the unique genomic, epigenetic, and/or transcriptomic profile of an individual's cancer. Personalized cancer treatment builds upon conventional therapeutic regimens used to treat cancer based only on the gross classification of the cancer, e.g., treating all breast cancer patients with a first therapy and all lung cancer patients with a second therapy. This field was borne out of many observations that different patients diagnosed with the same type of cancer, e.g., breast cancer, responded very differently to common treatment regimens. Over time, researchers have identified genomic, epigenetic, and transcriptomic markers that improve predictions as to how an individual cancer will respond to a particular treatment modality.

There is growing evidence that cancer patients who receive therapy guided by their genetics have better outcomes. For example, studies have shown that targeted therapies result in significantly improved progression-free cancer survival. See, e.g., Radovich M. et al., Oncotarget, 7 (35): 56491-500 (2016). Similarly, reports from the IMPACT trial—a large (n=1307) retrospective analysis of consecutive, prospectively molecularly profiled patients with advanced cancer who participated in a large, personalized medicine trial—indicate that patients receiving targeted therapies matched to their tumor biology had a response rate of 16.2%, as opposed to a response rate of 5.2% for patients receiving non-matched therapy. Tsimberidou A M et al., ASCO 2018, Abstract LBA2553 (2018).

In fact, therapy targeted to specific genomic alterations is already the standard of care in several tumor types, e.g., as suggested in the National Comprehensive Cancer Network (NCCN) guidelines for melanoma, colorectal cancer, and non-small cell lung cancer. In practice, implementation of these targeted therapies requires determining the status of the diagnostic marker in each eligible cancer patient. While this can be accomplished for the few, well known mutations associated with treatment recommendations in the NCCN guidelines using individual assays or small next generation sequencing (NGS) panels, the growing number of actionable genomic alterations and increasing complexity of diagnostic classifiers necessitates a more comprehensive evaluation of each patient's cancer genome, epigenome, and/or transcriptome.

For instance, some evidence suggests that use of combination therapies where each component is matched to an actionable genomic alteration holds the greatest potential for treating individual cancers. To this point, a retroactive study of cancer patients treated with one or more therapeutic regimens revealed that patients who received therapies matched to a higher percentage of their genomic alterations experienced a greater frequency of stable disease (e.g., a longer time to recurrence), longer time to treatment failure, and greater overall survival. Wheeler J J et al., Cancer Res., 76:3690-701 (2016). Thus, comprehensive evaluation of each cancer patient's genome, epigenome, and/or transcriptome should maximize the benefits provided by precision oncology, by facilitating more fine-tuned combination therapies, use of novel off-label drug indications, and/or tissue agnostic immunotherapy. See, for example, Schwaederle M. et al., J Clin Oncol., 33 (32): 3817-25 (2015); Schwaederle M. et al., JAMA Oncol., 2 (11): 1452-59 (2016); and Wheler J J et al., Cancer Res., 76 (13): 3690-701 (2016). Further, the use of comprehensive next generation sequencing analysis of cancer genomes facilitates better access and a larger patient pool for clinical trial enrollment. Coyne G O et al., Curr. Probl. Cancer, 41 (3): 182-93 (2017); and Markman M., Oncology, 31(3):158, 168.

The use of large NGS genomic analysis is growing in order to address the need for more comprehensive characterization of an individual's cancer genome. See, for example, Fernandes G S et al., Clinics, 72(10):588-94. Recent studies indicate that of the patients for which large NGS genomic analysis is performed, 30-40% then receive clinical care based on the assay results, which is limited by at least the identification of actionable genomic alterations, the availability of medication for treatment of identified actionable genomic alterations, and the clinical condition of the subject. Sec, Ross J S et al., JAMA Oncol., 1(1):40-49 (2015); Ross J S et al., Arch. Pathol. Lab Med., 139:642-49 (2015); Hirshfield K M et al., Oncologist, 21(11):1315-25 (2016); and Groisberg R. et al., Oncotarget, 8:39254-67 (2017).

However, these large NGS genomic analyses are conventionally performed on solid tumor samples. For instance, each of the studies referenced in the paragraph above performed NGS analysis of FFPE tumor blocks from patients. Solid tissue biopsies remain the gold standard for diagnosis and identification of predictive biomarkers because they represent well-known and validated methodologies that provide a high degree of accuracy. Nevertheless, there are significant limitations to the use of solid tissue material for large NGS genomic analyses of cancers. For example, tumor biopsies are subject to sampling bias caused by spatial and/or temporal genetic heterogeneity, e.g., between two regions of a single tumor and/or between different cancerous tissues (such as between primary and metastatic tumor sites or between two different primary tumor sites). Such intertumor or intratumor heterogeneity can cause subclonal or emerging mutations to be overlooked when using localized tissue biopsies, with the potential for sampling bias to be exacerbated over time as subclonal populations further evolve and/or shift in predominance.

Additionally, the acquisition of solid tissue biopsies often requires invasive surgical procedures, e.g., when the primary tumor site is located at an internal organ. These procedures can be expensive, time consuming, and carry a significant risk to the patient, e.g., when the patient's health is poor and may not be able to tolerate invasive medical procedures and/or the tumor is located in a particularly sensitive or inoperable location, such as in the brain or heart. Further, the amount of tissue, if any, that can be procured depends on multiple factors, including the location of the tumor, the size of the tumor, the fragility of the patient, and the risk of comorbidities related to biopsies, such as bleeding and infections. For instance, recent studies report that tissue samples in a majority of advanced non-small cell lung cancer patients are limited to small biopsies, and cannot be obtained at all in up to 31% of patients. Ilie and Hofman, Transl. Lung Cancer Res., 5(4):420-23 (2016). Even when a tissue biopsy is obtained, the sample may be too scant for comprehensive testing.

Further, the method of tissue collection, preservation (e.g., formalin fixation), and/or storage of tissue biopsies can result in sample degradation and variable quality DNA. This, in turn, leads to inaccuracies in downstream assays and analysis, including next-generation sequencing (NGS) for the identification of biomarkers. Ilie and Hofman, Transl Lung Cancer Res., 5(4):420-23 (2016).

In addition, the invasive nature of the biopsy procedure, the time and cost associated with obtaining the sample, and the compromised state of cancer patients receiving therapy render repeat testing of cancerous tissues impracticable, if not impossible. As a result, solid tissue biopsy analysis is not amenable to many monitoring schemes that would benefit cancer patients, such as disease progression analysis, treatment efficacy evaluation, disease recurrence monitoring, and other techniques that require data from several time points.

Liquid biopsies offer several advantages over conventional solid tissue biopsy analysis for precision oncology. For instance, because bodily fluids can be collected in a minimally invasive or non-invasive fashion, sample collection is simpler, faster, safer, and less expensive than solid tumor biopsies. Such methods require only small amounts of sample (e.g., 10 mL or less of whole blood per biopsy) and reduce the discomfort and risk of complications experienced by patients during conventional tissue biopsies. In fact, liquid biological samples can be collected with limited or no assistance from medical professionals and can be performed at almost any location. Further, liquid biological samples can be collected from any patient, regardless of the location of their cancer, their overall health, and any previous biopsy collection. This allows for analysis of the cancer genome of patients from which a solid tumor sample cannot be easily and/or safely obtained. In addition, because cell-free DNA in the bodily fluids arise from many different types of tissues in the patient, the genomic alterations present in the pool of cell-free DNA are representative of various different clonal sub-populations of the cancerous tissue of the subject and of all tumors in a patient having more than one tumor, facilitating a more comprehensive analysis of the cancerous genome of the subject than is possible from one or more sections of a single solid tumor sample.

Liquid biopsies also enable serial genetic testing prior to cancer detection, during the early stages of cancer progression, throughout the course of treatment, and during remission, e.g., to monitor for disease recurrence. The ability to conduct serial testing via non-invasive liquid biopsies throughout the course of disease could prove beneficial for many patients, e.g., through monitoring patient response to therapies, the emergence of new actionable genomic alterations, and/or drug-resistance alterations. These types of information allow medical professionals to more quickly tailor and update therapeutic regimens, e.g., facilitating more timely intervention in the case of disease progression. See, e.g., Ilie and Hofman, Transl. Lung Cancer Res., 5(4):420-23 (2016).

Nevertheless, while liquid biopsies are promising tools for improving outcomes using precision oncology, there are significant challenges specific to the use of cell-free DNA for evaluation of a subject's cancer genome. For instance, there is a highly variable signal-to-noise ratio from one liquid biological sample to the next. This occurs because cfDNA originates from a variety of different cells in a subject, both healthy and diseased. Depending on the stage and type of cancer in any particular subject, the fraction of cfDNA fragments originating from cancerous cells (the “tumor fraction” or “ctDNA fraction” of the sample/subject) can range from almost 0% to well over 50%. Other factors, including tumor type and mutation profile, can also impact the amount of DNA released from cancerous tissues. For instance, cfDNA clearance through the liver and kidneys is affected by a variety of factors, including renal dysfunction or other tissue damaging factors (e.g., chemotherapy, surgery, and/or radiotherapy).

This, in turn, leads to problems detecting and/or validating cancer-specific genomic alterations in a liquid sample. This is particularly true during early stages of the disease-when cancer therapies have much higher success rates-because the tumor fraction in the patient is lowest at this point. Thus, early stage cancer patients can have ctDNA fractions below the limit of detection (LOD) for one or more informative genomic alterations, limiting clinical utility because of the risk of false negatives and/or providing an incomplete picture of the cancer genome of the patient. Further, because cancers, and even individual tumors, can be clonally diverse, actionable genomic alterations that arise in only a subset of clonal populations are diluted below the overall tumor fraction of the sample, further frustrating attempts to tailor combination therapies to the various actionable mutations in the patient's cancer genome. Consequently, most studies using liquid biopsy samples to date have focused on late stage patients for assay validation and research.

Another challenge associated with liquid biopsies is the accurate determination of tumor fraction in a sample. This difficulty arises from at least the heterogeneity of cancers and the increased frequency of large chromosomal duplications and deletions found in cancers. As a result, the frequency of genomic alterations from cancerous tissues varies from locus to locus based on at least (i) their prevalence in different subclonal populations of the subject's cancer, and (ii) their location within the genome, relative to large chromosomal copy number variations. The difficulty in accurately determining the tumor fraction of liquid biological samples affects accurate measurement of various cancer features shown to have diagnostic value for the analysis of solid tumor biopsies. These include allelic ratios, copy number variations, overall mutational burden, frequency of abnormal methylation patterns, etc., all of which are correlated with the percentage of DNA fragments that arise from cancerous tissue, as opposed to healthy tissue.

Altogether, these factors result in highly variable concentrations of ctDNA—from patient to patient and possibly from locus to locus—that confound accurate measurement of disease indicators and actionable genomic alterations. Further, the quantity and quality of cfDNA obtained from liquid biopsy samples are highly dependent on the particular methodology for collecting the samples, storing the samples, sequencing the samples, and standardizing the sequencing data.

While validation studies of existing liquid biopsy assays have shown high sensitivity and specificity, few studies have corroborated results with orthogonal methods, or between particular testing platforms, e.g., different NGS technologies and/or targeted panel sequencing versus whole genome/exome sequence. Reports of liquid biopsy-based studies are limited by comparison to non-comprehensive tissue testing algorithms including Sanger sequencing, small NGS hotspot panels, polymerase chain reaction (PCR), and fluorescent in situ hybridization (FISH), which may not contain all NCCN guideline genes in their reportable range, thus suffering in comparison to a more comprehensive liquid biopsy assay.

The information disclosed in this Background section is only for enhancement of understanding of the general background of the invention and should not be taken as an acknowledgement or any form of suggestion that this information forms the prior art already known to a person skilled in the art.

### SUMMARY

Given the above background, there is a need in the art for improved methods and systems for cancer screening. There is also a need in the art for improved methods and systems for estimating a circulating tumor fraction in a liquid biopsy sample. The present disclosure solves these and other needs in the art by providing methods for estimating circulating tumor fraction, cancer detection, and cancer monitoring using whole genome methylation sequencing.

For example, in one aspect, the present disclosure provides methods and systems for monitoring a cancer condition of a test subject. Briefly, in some embodiments, a liquid biopsy sample is obtained from the test subject at a second time point, occurring after a first time point. The liquid biopsy sample includes a plurality of cell-free DNA fragments. The plurality of cell-free DNA fragments are sequenced, in a whole genome methylation sequencing reaction (e.g., in a low-pass whole genome sequencing reaction at an average unique sequencing depth of less than 3× across the entire genome of the species of the test subject), thereby obtaining a set of nucleic acid sequences, where each respective nucleic acid sequence in the set of nucleic acid sequences includes a methylation pattern for a corresponding cell-free DNA fragment in the plurality of cell-free DNA fragments. Each respective nucleic acid sequence, in the set of nucleic acid sequences, is mapped to a location on a reference genome for the species of the subject. A plurality of methylation metrics for the liquid biopsy sample are determined based on at least (i) the methylation pattern of each respective nucleic acid sequence in the set of nucleic acid sequences, and (ii) the location in the reference genome that each respective nucleic acid sequence in the set of nucleic acid sequence was mapped to. A circulating tumor fraction of the test subject at the second time point is estimated using the plurality of methylation metrics for the liquid biopsy sample, and the estimate of the circulating tumor fraction of the test subject at the second time point is compared to an estimate of the circulating tumor fraction for the test subject at the first time point, thereby monitoring the cancer condition of the test subject.

In another aspect, the present disclosure provides methods and systems for characterizing a cancer condition of a test subject. A liquid biopsy sample is obtained from the test subject. The liquid biopsy sample includes a first and a second plurality of cell-free DNA fragments. The first plurality of cell-free DNA fragments is sequenced in a whole genome methylation sequencing reaction (e.g., in a low-pass whole genome sequencing reaction at an average unique sequencing depth of less than 3× across the entire genome of the species of the test subject), thereby obtaining a first set of nucleic acid sequences, where each respective nucleic acid sequence in the first set of nucleic acid sequences includes a methylation pattern for a corresponding cell-free DNA fragment in the first plurality of cell-free DNA fragments. The second plurality of the cell-free DNA fragments is sequenced, in a targeted sequencing reaction, at an average unique sequencing depth of at least 50× across the targeted panel, thereby obtaining a second set of sequences corresponding to the second plurality of cell-free DNA fragments. The circulating tumor fraction of the test subject is estimated based on the methylation pattern of nucleic acid sequences in the first set of nucleic acid sequences, and the circulating tumor fraction estimate for the test subject is used in the analysis of the second set of sequences to characterize the cancer condition in the test subject.

In another aspect, the present disclosure provides methods and systems for determining an extent of minimal residual disease (MRD) in a test subject following cancer therapy. A liquid biopsy sample is obtained from the test subject following the completion of a cancer therapy regimen. The liquid biopsy sample includes cell-free DNA fragments. A plurality of the cell-free DNA fragments are sequenced, in a whole genome methylation sequencing reaction (e.g., in a low-pass whole genome sequencing reaction at an average unique sequencing depth of less than 3× across the entire genome of the species of the test subject), thereby obtaining a set of nucleic acid sequences, where each respective nucleic acid sequence in the set of nucleic acid sequences includes a methylation pattern for a corresponding cell-free DNA fragment in the plurality of cell-free DNA fragments. Each respective nucleic acid sequence, in the set of nucleic acid sequences, is mapped to a location in a reference genome for the species of the subject. A plurality of methylation metrics are determined for the liquid biopsy sample based on at least (i) the methylation pattern of each respective nucleic acid sequence in the set of nucleic acid sequences, and (ii) the location in the reference genome that each respective nucleic acid sequence in the set of nucleic acid sequence was mapped to. A circulating tumor fraction of the test subject at the second time point is estimated using the plurality of methylation metrics for the liquid biopsy sample. The extent of MRD in the test subject is then determined based on the estimate of the circulating tumor fraction of the test subject.

In another aspect, the present disclosure provides methods and systems for monitoring the efficacy of a cancer treatment. A liquid biopsy sample is obtained from the test subject at one or more times during a cancer therapy regimen. The liquid biopsy sample(s) include cell-free DNA fragments. A plurality of the cell-free DNA fragments from a respective liquid biopsy sample obtained at a respective time in the one or more times are sequenced, in a whole genome methylation sequencing reaction (e.g., in a low-pass whole genome sequencing reaction at an average unique sequencing depth of less than 3× across the entire genome of the species of the test subject), where each respective nucleic acid sequence in the set of nucleic acid sequences includes a methylation pattern for a corresponding cell-free DNA fragment in the plurality of cell-free DNA fragments. Each respective nucleic acid sequence, in the set of nucleic acid sequences, is mapped to a location in a reference genome for the species of the subject. A plurality of methylation metrics are determined for the liquid biopsy sample based on at least (i) the methylation pattern of each respective nucleic acid sequence in the set of nucleic acid sequences, and (ii) the location in the reference genome that each respective nucleic acid sequence in the set of nucleic acid sequence was mapped to. A circulating tumor fraction of the test subject at the respective time point is estimated using the plurality of methylation metrics for the liquid biopsy sample. The efficacy of the cancer therapy regimen is then evaluated based on the estimate of the circulating tumor fraction of the test subject, e.g., by comparing it to an estimate of the circulating tumor fraction of the test subject at an earlier point in time during the cancer therapy regimen and/or prior to starting the cancer therapy regimen. Generally, a reduction in the circulating tumor fraction of the subject during the cancer therapy regimen is an indication that the cancer therapy regimen is effective.

In another aspect, the present disclosure provides methods and systems for estimating the circulating tumor fraction of a test subject. A dataset, in electronic form, is obtained, the data set including a set of nucleic acid sequences from a whole genome methylation sequencing of a plurality of cell-free DNA fragments from a liquid biopsy sample obtained from the test subject, where each respective nucleic acid sequence in the set of nucleic acid sequences includes a methylation pattern for a corresponding cell-free DNA fragment in the plurality of cell-free DNA fragments. Each respective nucleic acid sequence, in the set of nucleic acid sequences, is mapped to a location in a reference genome for the species of the subject. A plurality of methylation metrics is determined for the liquid biopsy sample based on at least (i) the methylation pattern of each respective nucleic acid sequence in the set of nucleic acid sequences, and (ii) the location in the reference genome that each respective nucleic acid sequence in the set of nucleic acid sequence was mapped to. A circulating tumor fraction of the test subject is then estimated using the plurality of methylation metrics for the liquid biopsy sample.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIGS. 1A, 1B, 1C, and 1D collectively illustrate a block diagram of an example computing device for early cancer detection, cancer monitoring, and circulating tumor fraction estimation using liquid biopsy whole genome methylation sequencing data, in accordance with some embodiments of the present disclosure.

FIG. 2A illustrates an example workflow for generating a clinical report based on information generated from analysis of one or more patient specimens, in accordance with some embodiments of the present disclosure.

FIG. 2B illustrates an example of a distributed diagnostic environment for collecting and evaluating patient data for the purpose of precision oncology, in accordance with some embodiments of the present disclosure.

FIG. 3 provides an example flow chart of processes and features for liquid biopsy sample collection and analysis for use in precision oncology, in accordance with some embodiments of the present disclosure.

FIGS. 4A and 4B, 4C, 4D, 4E, and 4F collectively illustrate an example bioinformatics pipeline for precision oncology. FIG. 4A provides an overview flow chart of processes and features in a bioinformatics pipeline, in accordance with some embodiments of the present disclosure. FIG. 4B provides an overview of a bioinformatics pipeline executed with either a liquid biopsy sample alone or a liquid biopsy sample and a matched normal sample. FIG. 4C illustrates that paired-end reads from tumor and normal isolates are zipped and stored separately under the same order identifier, in accordance with some embodiments of the present disclosure. FIG. 4D illustrates quality correction for FASTQ files, in accordance with some embodiments of the present disclosure. FIG. 4E illustrates steps for obtaining tumor and normal BAM alignment files, in accordance with some embodiments of the present disclosure. FIG. 4F provides an overview flow chart of methods for early cancer detection, cancer monitoring, and circulating tumor fraction estimation using liquid biopsy whole genome methylation sequencing data (e.g., low-pass whole genome methylation sequencing data), in accordance with some embodiments of the present disclosure.

FIG. 5 provides a flow chart of processes and features for early cancer detection and circulating tumor fraction estimation using liquid biopsy whole genome methylation sequencing data (e.g., low-pass whole genome methylation sequencing data), in accordance with some embodiments of the present disclosure.

FIG. 6 provides a flow chart of processes and features for characterizing a cancer condition using liquid biopsy sequencing data, in accordance with some embodiments of the present disclosure.

FIG. 7 provides a flow chart of processes and features for evaluating minimal residual disease (MRD) using liquid biopsy whole genome methylation sequencing data (e.g., low-pass whole genome methylation sequencing data), in accordance with some embodiments of the present disclosure.

FIG. 8 provides a flow chart of processes and features for early cancer detection and circulating tumor fraction estimation using liquid biopsy whole genome methylation sequencing data (e.g., low-pass whole genome methylation sequencing data), in accordance with some embodiments of the present disclosure.

FIG. 9 provides a flow chart of processes and features for estimating a circulating tumor fraction of a liquid biopsy assay using whole genome methylation sequencing data, in accordance with some embodiments of the present disclosure.

FIG. 10A illustrates a plot of the number of DNA fragment sequences determined to be significantly unlikely to be derived from non-cancerous tissue based on their methylation patterns in in silico samplings of 30,000 unique sequences that were sampled from either a mix of cancerous and non-cancerous samples (open circles) or non-cancerous samples only (closed circles).

FIG. 10B illustrates a plot of the number of DNA fragment sequences determined to be significantly unlikely to be derived from non-cancerous tissue based on their methylation patterns in in silico samplings of 150,000 unique sequences that were sampled from either a mix of cancerous and non-cancerous samples (open circles) or non-cancerous samples only (closed circles).

FIG. 11 illustrates a block diagram of an ensemble model for detecting cancer and/or estimating a circulating tumor fraction of a liquid biopsy sample based on whole genome methylation sequencing of cfDNA in the liquid biopsy sample, in accordance with various embodiments of the present disclosure.

FIG. 12 illustrates a comparison between (i) circulating tumor fraction estimations prepared by analysis of copy number variation (x-axis), and (ii) circulating tumor fraction estimations prepared by analysis of methylation patterns (y-axis), based on sequencing of cfDNA in liquid biopsy samples of subjects with and without cancer.

FIG. 13 illustrates an example matrix of normalized probabilities for a bivariate kernel density estimation (KDE), prepared as described in Example 4.

FIG. 14 illustrates a receiver operating characteristic curve for the performance of four component cancer classifiers, as described in Example 6.

Like reference numerals refer to corresponding parts throughout the several views of the drawings.

### DETAILED DESCRIPTION

### Introduction

As described above, conventional cancer screening methods are expensive, inefficient, invasive, and time consuming. For instance, breast cancer, colon cancer, and cervical cancer are each conventionally screened for using different, invasive screening methodologies that potentially require a patient to make three separate trips to a clinical environment for appointments in three separate departments. Advantageously, the present disclosure provides methods and systems for minimally invasive, liquid biopsy-based cancer screening methodologies capable of screening for many different cancer types in a single assay.

Moreover, in some embodiments, unlike conventional liquid biopsy assays that rely on high-depth whole genome, whole exome, or targeted panel sequencing, the methods and systems described herein use data from lower-cost low-pass whole genome methylation sequencing reactions. Specifically, low-pass whole genome methylation sequencing data can be used to classify a cancer status of a subject, monitor cancer therapy, or monitor for cancer recurrence, despite generating very few reads at each genomic locus.

Further, as described above, conventional liquid biopsy assays suffer from inaccurate determination of the tumor fraction of a liquid biopsy sample. As a result, these assays can perform poorly, particularly at lower tumor fractions characteristic of early stage cancers. Because early detection and treatment of cancer is associated with improved clinical outcomes, it is important to improve the performance of these assays at lower tumor fractions. Advantageously, the present disclosure provides methods and systems for improving the performance of liquid biopsy assays by facilitating more accurate determination of the tumor fraction of the sample using whole genome methylation sequencing data (e.g., low-pass whole genome sequencing data) generated from the sample. For instance, in some embodiments, both whole genome methylation sequencing and high pass sequencing (e.g., targeted-panel sequencing) are performed on aliquots of a sample, and data from the whole genome methylation sequencing reaction is used alone or in combination with sequencing data from the targeted panel high-pass sequencing reaction (e.g., a high-pass targeted panel sequencing reaction), e.g., to provide an improved estimate of the tumor fraction of the sample. In turn, identification of various genomic features from the high-pass whole genome sequencing data, such as somatic variants, variant allele fractions, and tumor heterogeneity, is improved. In some embodiments, both high-pass genomic sequencing (e.g., targeted panel sequencing) and low-pass methylation sequencing are performed on aliquots of a sample, and data from the low-pass methylation sequencing reaction is used alone or in combination with sequencing data from the high-pass sequencing reaction (e.g., a high-pass targeted panel sequencing reaction), e.g., to provide an improved estimate of the tumor fraction of the sample. In turn, identification of various genomic features from the high-pass whole genome sequencing data, such as somatic variants, variant allele fractions, and tumor heterogeneity, is improved.

The identification of actionable genomic alterations in a patient's cancer genome is a difficult and computationally demanding problem. For instance, the determination of various prognostic metrics useful for precision oncology, such as variant allelic ratio, copy number variation, tumor mutational burden, microsatellite instability status, etc., requires analysis of hundreds of millions to billions, of sequenced nucleic acid bases. An example of a typical bioinformatics pipeline established for this purpose includes at least five stages of analysis: assessment of the quality of raw next generation sequencing data, generation of collapsed nucleic acid fragment sequences and alignment of such sequences to a reference genome, detection of structural variants in the aligned sequence data, annotation of identified variants, and visualization of the data. Wadapurkar and Vyas, Informatics in Medicine Unlocked, 11:75-82 (2018), the content of which is hereby incorporated by reference, in its entirety, for all purposes. Each one of these steps is computationally taxing in its own right.

For instance, the overall temporal and spatial computational complexity of simple global and local pairwise sequence alignment algorithms are quadratic in nature (i.e., second order problems), that increase rapidly as a function of the size of the nucleic acid sequences (n and m) being compared. Specifically, the temporal and spatial complexities of these sequence alignment algorithms can be estimated as O(mn), where O is the upper bound on the asymptotic growth rate of the algorithm, n is the number of bases in the first nucleic acid sequence, and m is the number of bases in the second nucleic acid sequence. Baichoo and Ouzounis, BioSystems, 156-157:72-85 (2017), the content of which is hereby incorporated by reference, in its entirety, for all purposes. Given that the human genome contains more than 3 billion bases, these alignment algorithms are extremely computationally taxing, especially when used to analyze next generation sequencing (NGS) data, which can generate more than 3 billion sequence reads per reaction.

This is particularly true when performed in the context of a liquid biopsy assay, because liquid biological samples contain a complex mixture of short DNA fragments originating from many different germline (e.g., healthy) and diseased (e.g., cancerous) tissues. Thus, the cellular origins of the sequence reads are unknown, and the sequence signals originating from cancerous cells, which may constitute multiple sub-clonal populations, must be computationally deconvolved from signals originating from germline and hematopoietic origins, in order to provide relevant information about the subject's cancer. Thus, in addition to the computationally taxing processes required to align sequence reads to a human genome, there is a computation problem of determining whether a particular abnormal signal, e.g., one or more sequence reads corresponding to a genomic alteration, (i) is not an artifact, and (ii) originated from a cancerous source in the subject. This is increasingly difficult during the early stages of cancer—when treatment is presumably most effective—when only small amounts of ctDNA are diluted by germline and hematopoietic DNA.

### Definitions

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the terms “control,” “control sample,” “reference,” “reference sample,” “normal,” and “normal sample” describe a sample from a non-diseased tissue. In some embodiments, such a sample is from a subject that does not have a particular condition (e.g., cancer). In other embodiments, such a sample is an internal control from a subject, e.g., who may or may not have the particular disease (e.g., cancer), but is from a healthy tissue of the subject. For example, where a liquid or solid tumor sample is obtained from a subject with cancer, an internal control sample may be obtained from a healthy tissue of the subject, e.g., a white blood cell sample from a subject without a blood cancer or a solid germline tissue sample from the subject. Accordingly, a reference sample can be obtained from the subject or from a database, e.g., from a second subject who does not have the particular disease (e.g., cancer).

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

As used herein, the terms “cancer state” or “cancer condition” refer to a characteristic of a cancer patient's condition, e.g., a diagnostic status, a type of cancer, a location of cancer, a primary origin of a caner, a cancer stage, a cancer prognosis, and/or one or more additional characteristics of a cancer (e.g., tumor characteristics such as morphology, heterogeneity, size, etc.). In some embodiments, one or more additional personal characteristics of the subject are used further describe the cancer state or cancer condition of the subject, e.g., age, gender, weight, race, personal habits (e.g., smoking, drinking, diet), other pertinent medical conditions (e.g., high blood pressure, dry skin, other diseases), current medications, allergies, pertinent medical history, current side effects of cancer treatments and other medications, etc.

As used herein, the term “liquid biopsy” sample refers to any sample taken from a subject, which can reflect a biological state associated with the subject, and that includes cell-free DNA. Examples of liquid biopsy samples include, but are not limited to, blood, whole blood, plasma, serum, urine, cerebrospinal fluid, fecal, saliva, sweat, tears, pleural fluid, pericardial fluid, or peritoneal fluid of the subject. A liquid biopsy sample can include any tissue or material derived from a living or dead subject. A liquid biopsy sample can be a cell-free sample. A liquid biopsy sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof.

As used herein, the term “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope.

As used herein, the term “locus” refers to a position (e.g., a site) within a genome, e.g., on a particular chromosome. In some embodiments, a locus refers to a single nucleotide position, on a particular chromosome, within a genome. In some embodiments, a locus refers to a group of nucleotide positions within a genome. In some instances, a locus is defined by a mutation (e.g., substitution, insertion, deletion, inversion, or translocation) of consecutive nucleotides within a cancer genome. In some instances, a locus is defined by a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome.

As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

As used herein, the term “base pair” or “bp” refers to a unit consisting of two nucleobases bound to each other by hydrogen bonds. Generally, the size of an organism's genome is measured in base pairs because DNA is typically double stranded. However, some viruses have single-stranded DNA or RNA genomes.

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (CNV), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject. For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘mutations.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species.

As used herein, the term “variant allele frequency,” “VAF,” “allelic fraction,” or “AF” refers to the number of times a variant or mutant allele was observed (e.g., a number of reads supporting a candidate variant allele) divided by the total number of times the position was sequenced (e.g., a total number of reads covering a candidate locus).

As used herein, the term “germline variants” refers to genetic variants inherited from maternal and paternal DNA. Germline variants may be determined through a matched tumor-normal calling pipeline.

As used herein, the term “somatic variants” refers to variants arising as a result of dysregulated cellular processes associated with neoplastic cells, e.g., a mutation. Somatic variants may be detected via subtraction from a matched normal sample.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “insertions and deletions” or “indels” refers to a variant resulting from the gain or loss of DNA base pairs within an analyzed region.

As used herein, the term “copy number variation” or “CNV” refers to the process by which large structural changes in a genome associated with tumor aneuploidy and other dysregulated repair systems are detected. These processes are used to detect large scale insertions or deletions of entire genomic regions. CNV is defined as structural insertions or deletions greater than a certain base pair (“bp”) in size, such as 500 bp.

As used herein, the term “gene fusion” refers to the product of large scale chromosomal aberrations resulting in the creation of a chimeric protein. These expressed products can be non-functional, or they can be highly over or underactive. This can cause deleterious effects in cancer such as hyper-proliferative or anti-apoptotic phenotypes.

As used herein, the term “loss of heterozygosity” refers to the loss of one copy of a segment (e.g., including part or all of one or more genes) of the genome of a diploid subject (e.g., a human) or loss of one copy of a sequence encoding a functional gene product in the genome of the diploid subject, in a tissue, e.g., a cancerous tissue, of the subject. As used herein, when referring to a metric representing loss of heterozygosity across the entire genome of the subject, loss of heterozygosity is caused by the loss of one copy of various segments in the genome of the subject. Loss of heterozygosity across the entire genome may be estimated without sequencing the entire genome of a subject, and such methods for such estimations based on gene panel targeting-based sequencing methodologies are described in the art. Accordingly, in some embodiments, a metric representing loss of heterozygosity across the entire genome of a tissue of a subject is represented as a single value, e.g., a percentage or fraction of the genome. In some cases a tumor is composed of various sub-clonal populations, each of which may have a different degree of loss of heterozygosity across their respective genomes. Accordingly, in some embodiments, loss of heterozygosity across the entire genome of a cancerous tissue refers to an average loss of heterozygosity across a heterogeneous tumor population. As used herein, when referring to a metric for loss of heterozygosity in a particular gene, e.g., a DNA repair protein such as a protein involved in the homologous DNA recombination pathway (e.g., BRCA1 or BRCA2), loss of heterozygosity refers to complete or partial loss of one copy of the gene encoding the protein in the genome of the tissue and/or a mutation in one copy of the gene that prevents translation of a full-length gene product, e.g., a frameshift or truncating (creating a premature stop codon in the gene) mutation in the gene of interest. In some cases a tumor is composed of various sub-clonal populations, each of which may have a different mutational status in a gene of interest. Accordingly, in some embodiments, loss of heterozygosity for a particular gene of interest is represented by an average value for loss of heterozygosity for the gene across all sequenced sub-clonal populations of the cancerous tissue. In other embodiments, loss of heterozygosity for a particular gene of interest is represented by a count of the number of unique incidences of loss of heterozygosity in the gene of interest across all sequenced sub-clonal populations of the cancerous tissue (e.g., the number of unique frame-shift and/or truncating mutations in the gene identified in the sequencing data).

As used herein, the term “microsatellites” refers to short, repeated sequences of DNA. The smallest nucleotide repeated unit of a microsatellite is referred to as the “repeated unit” or “repeat unit.” In some embodiments, the stability of a microsatellite locus is evaluated by comparing some metric of the distribution of the number of repeated units at a microsatellite locus to a reference number or distribution.

As used herein, the term “microsatellite instability” or “MSI” refers to a genetic hypermutability condition associated with various cancers that results from impaired DNA mismatch repair (MMR) in a subject. Among other phenotypes, MSI causes changes in the size of microsatellite loci, i.e., a change in the number of repeated units at microsatellite loci, during DNA replication. Accordingly, the size of microsatellite repeats is varied in MSI cancers as compared to the size of the corresponding microsatellite repeats in the germline of a cancer subject. The term “Microsatellite Instability-High” or “MSI-H” refers to a state of a cancer (e.g., a tumor) that has a significant MMR defect, resulting in microsatellite loci with significantly different lengths than the corresponding microsatellite loci in normal cells of the same individual. The term “Microsatellite Stable” or “MSS” refers to a state of a cancer (e.g., a tumor) without significant MMR defects, such that there is no significant difference between the lengths of the microsatellite loci in cancerous cells and the lengths of the corresponding microsatellite loci in normal (e.g., non-cancerous) cells in the same individual. The term “Microsatellite Equivocal” or “MSE” refers to a state of a cancer (e.g., a tumor) having an intermediate microsatellite length phenotype, that cannot be clearly classified as MSI-H or MSS based on statistical cutoffs used to define those two categories.

As used herein, the term “gene product” refers to an RNA (e.g., mRNA or miRNA) or protein molecule transcribed or translated from a particular genomic locus, e.g., a particular gene. The genomic locus can be identified using a gene name, a chromosomal location, or any other genetic mapping metric.

As used herein, the terms “expression level,” “abundance level,” or simply “abundance” refers to an amount of a gene product, (an RNA species, e.g., mRNA or miRNA, or protein molecule) transcribed or translated by a cell, or an average amount of a gene product transcribed or translated across multiple cells. When referring to mRNA or protein expression, the term generally refers to the amount of any RNA or protein species corresponding to a particular genomic locus, e.g., a particular gene. However, in some embodiments, an expression level can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used herein, the term “relative abundance” refers to a ratio of a first amount of a compound measured in a sample, e.g., a gene product (an RNA species, e.g., mRNA or miRNA, or protein molecule) or nucleic acid fragments having a particular characteristic (e.g., aligning to a particular locus or encompassing a particular allele), to a second amount of a compound measured in a second sample. In some embodiments, relative abundance refers to a ratio of an amount of species of a compound to a total amount of the compound in the same sample. For instance, a ratio of the amount of mRNA transcripts encoding a particular gene in a sample (e.g., aligning to a particular region of the exome) to the total amount of mRNA transcripts in the sample. In other embodiments, relative abundance refers to a ratio of an amount of a compound or species of a compound in a first sample to an amount of the compound of the species of the compound in a second sample. For instance, a ratio of a normalized amount of mRNA transcripts encoding a particular gene in a first sample to a normalized amount of mRNA transcripts encoding the particular gene in a second and/or reference sample.

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “genetic sequence” refers to a recordation of a series of nucleotides present in a subject's RNA or DNA as determined by sequencing of nucleic acids from the subject.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “read segment” refers to any form of nucleotide sequence read including the raw sequence reads obtained directly from a nucleic acid sequencing technique or from a sequence derived therefrom, e.g., an aligned sequence read, a collapsed sequence read, or a stitched sequence read.

As used herein, the term “read count” refers to the total number of nucleic acid reads generated, which may or may not be equivalent to the number of nucleic acid molecules generated, during a nucleic acid sequencing reaction.

As used herein, the term “read-depth,” “sequencing depth,” or “depth” can refer to a total number of unique nucleic acid fragments encompassing a particular locus or region of the genome of a subject that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is necessarily an integer, because it represents the actual sequencing depth for a particular locus. Alternatively, read-depth, sequencing depth, or depth can refer to a measure of central tendency (e.g., a mean or mode) of the number of unique nucleic acid fragments that encompass one of a plurality of loci or regions of the genome of a subject that are sequenced in a particular sequencing reaction. For example, in some embodiments, sequencing depth refers to the average depth of every locus across an arm of a chromosome, a targeted sequencing panel, an exome, or an entire genome. In such case, Y may be expressed as a fraction or a decimal, because it refers to an average coverage across a plurality of loci. When a mean depth is recited, the actual depth for any particular locus may be different than the overall recited depth. Metrics can be determined that provide a range of sequencing depths in which a defined percentage of the total number of locus fall. For instance, a range of sequencing depths within which 90% or 95%, or 99% of the loci fall.

As used herein, “whole genome sequencing,” including whole genome methylation sequencing, refers to a sequencing reaction performed on DNA derived from a genomic source (e.g., whether isolated directly from the cells, typically referred to as genomic DNA (“gDNA”), or from a liquid biopsy sample, typically referred to as cell-free DNA (“cfDNA”)) without enriching for any particular sequence(s). In contrast to targeted-panel sequencing, in which a set of target probes is used to enrich nucleic acids based on their sequence (e.g., to enrich for genomic regions of interest), whole genome sequencing does not use target probes. As understood by the skilled artisan, different sequencing technologies provide different sequencing depths. For instance, low-pass whole genome sequencing, including low-pass whole genome methylation sequencing, can refer to technologies that provide a sequencing depth of less than 5×, less than 4×, less than 3×, less than 2×, less than 1×, less than 0.75×, less than 0.5×, less than 0.25×, e.g., from about 0.1× to about 5×, from about 0.1× to about 4×, from about 0.1× to about 3×, from about 0.1× to about 2×, from about 0.25× to about 5×, from about 0.25× to about 4×, from about 0.25× to about 3×, from about 0.25× to about 2×, from about 0.5× to about 5×, from about 0.5× to about 4×, from about 0.5× to about 3×, or from about 0.5× to about 2×. Thus, while whole genome sequencing is performed without selecting for any particular genomic sequences, the sequencing results of such reactions do not necessarily include sequencing data for all portions of the genome. For instance, when a whole genome sequencing reaction is performed at an average sequencing depth of 0.75×, the entire genome is sequenced at an average depth of less than 1. Accounting for some overlap in such a sequencing reaction, less than 75% of the genome collectively is sequenced is a reaction performed at 0.75×. In fact, when whole genome sequencing is performed at very low sequencing depths, as little as 1 MB of the genome is collectively sequenced in the reaction. Accordingly, as used herein, whole genome sequencing relates to sequencing reactions that collectively sequence at least 1 MB of the genome of a subject. In some embodiments, whole genome sequencing relates to sequencing reactions that collectively sequence at least 2.5 MB, 5 MB, 10 MB, 15 MB, 20 MB, 25 MB, 30 MB, 40 MB, 50 MB, 100 MB, 250 MB, 500 MB, 750 MB, 1000 MB, 1500 MB, 2000 MB, or more of the genome of a subject.

As used herein, the term “sequencing breadth” refers to what fraction of a particular reference exome (e.g., human reference exome), a particular reference genome (e.g., human reference genome), or part of the exome or genome has been analyzed. Sequencing breadth can be expressed as a fraction, a decimal, or a percentage, and is generally calculated as (the number of loci analyzed/the total number of loci in a reference exome or reference genome). The denominator of the fraction can be a repeat-masked genome, and thus 100% can correspond to all of the reference genome minus the masked parts. A repeat-masked exome or genome can refer to an exome or genome in which sequence repeats are masked (e.g., sequence reads align to unmasked portions of the exome or genome). In some embodiments, any part of an exome or genome can be masked and, thus, sequencing breadth can be evaluated for any desired portion of a reference exome or genome. In some embodiments, “broad sequencing” refers to sequencing/analysis of at least 0.1% of an exome or genome.

As used herein, the term “sequencing probe” refers to a molecule that binds to a nucleic acid with affinity that is based on the expected nucleotide sequence of the RNA or DNA present at that locus.

As used herein, the term “targeted panel” or “targeted gene panel” refers to a combination of probes for sequencing (e.g., by next-generation sequencing) nucleic acids present in a biological sample from a subject (e.g., a tumor sample, liquid biopsy sample, germline tissue sample, white blood cell sample, or tumor or tissue organoid sample), selected to map to one or more loci of interest on one or more chromosomes. An example set of loci/genes useful for precision oncology, e.g., via solid or liquid biopsy assay, that can be analyzed using a targeted panel is described in Table 1. In some embodiments, in addition to loci that are informative for precision oncology, a targeted panel includes one or more probes for sequencing one or more of a locus associated with a different medical condition, a locus used for internal control purposes, or a locus from a pathogenic organism (e.g., an oncogenic pathogen).

As used herein, the term, “reference exome” refers to any sequenced or otherwise characterized exome, whether partial or complete, of any tissue from any organism or pathogen that may be used to reference identified sequences from a subject. Typically, a reference exome will be derived from a subject of the same species as the subject whose sequences are being evaluated. Example reference exomes used for human subjects as well as many other organisms are provided in the on-line genome browser hosted by the National Center for Biotechnology Information (“NCBI”). An “exome” refers to the complete transcriptional profile of an organism or pathogen, expressed in nucleic acid sequences. As used herein, a reference sequence or reference exome often is an assembled or partially assembled exomic sequence from an individual or multiple individuals. In some embodiments, a reference exome is an assembled or partially assembled exomic sequence from one or more human individuals. The reference exome can be viewed as a representative example of a species' set of expressed genes. In some embodiments, a reference exome comprises sequences assigned to chromosomes.

As used herein, the term “reference genome” refers to any sequenced or otherwise characterized genome, whether partial or complete, of any organism or pathogen that may be used to reference identified sequences from a subject. Typically, a reference genome will be derived from a subject of the same species as the subject whose sequences are being evaluated. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or pathogen, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). For a haploid genome, there can be only one nucleotide at each locus. For a diploid genome, heterozygous loci can be identified; each heterozygous locus can have two alleles, where either allele can allow a match for alignment to the locus.

As used herein, the term “bioinformatics pipeline” refers to a series of processing stages used to determine characteristics of a subject's genome or exome based on sequencing data of the subject's genome or exome. A bioinformatics pipeline may be used to determine characteristics of a germline genome or exome of a subject and/or a cancer genome or exome of a subject. In some embodiments, the pipeline extracts information related to genomic alterations in the cancer genome of a subject, which is useful for guiding clinical decisions for precision oncology, from sequencing results of a biological sample, e.g., a tumor sample, liquid biopsy sample, reference normal sample, etc., from the subject. Certain processing stages in a bioinformatics may be ‘connected,’ meaning that the results of a first respective processing stage is informative and/or essential for execution of a step in a second, downstream processing stage. For instance, in some embodiments, a bioinformatics pipeline includes a first respective processing stage for identifying genomic alterations that are unique to the cancer genome of a subject and a second respective processing stage that uses the quantity and/or identity of the identified genomic alterations to determine a metric that is informative for precision oncology, e.g., a tumor mutational burden. In some embodiments, the bioinformatics pipeline includes a reporting stage that generates a report of relevant and/or actionable information identified by upstream stages of the pipeline, which may or may not further include recommendations for aiding clinical therapy decisions.

As used herein, the term “limit of detection” or “LOD” refers to the minimal quantity of a feature that can be identified with a particular level of confidence. Accordingly, level of detection can be used to describe an amount of a substance that must be present in order for a particular assay to reliably detect the substance. A level of detection can also be used to describe a level of support needed for an algorithm to reliably identify a genomic alteration based on sequencing data. For example, a minimal number of unique sequence reads to support identification of a sequence variant such as a SNV.

As used herein, the term “BAM File” or “Binary file containing Alignment Maps” refers to a file storing sequencing data aligned to a reference sequence (e.g., a reference genome or exome). In some embodiments, a BAM file is a compressed binary version of a SAM (Sequence Alignment Map) file, which includes, for each of a plurality of unique sequence reads, an identifier for the sequence read, information about the nucleotide sequence, information about the alignment of the sequence to a reference sequence, and optionally metrics relating to the quality of the sequence read and/or the quality of the sequence alignment. While BAM files generally relate to files having a particular format, for simplicity they are used herein to simply refer to a file, of any format, containing information about a sequence alignment, unless specifically stated otherwise.

As used herein, the term “measure of central tendency” refers to a central or representative value for a distribution of values. Non-limiting examples of measures of central tendency include an arithmetic mean, weighted mean, midrange, midhinge, trimcan, geometric mean, geometric median, Winsorized mean, median, and mode of the distribution of values.

As used herein, the term “Positive Predictive Value” or “PPV” means the likelihood that a variant is properly called given that a variant has been called by an assay. PPV can be expressed as (number of true positives)/(number of false positives+ number of true positives).

As used herein, “data binning” refers to a data processing technique used to reduce the effects of minor observation errors. The original data values which fall into a given small interval, a bin, are replaced by a value representative of that interval. As used herein, the interval of a genomic bin represents a portion of a reference genome for the species of the subject, e.g., a portion of a human genome. In some embodiments, each bin in a plurality of bins represents a unique portion of the reference genome, although a plurality of bins need not cover the entire genome for the species of the subject. The size of a bins is application dependent. In some embodiments, each of the bins have equal sizes. That is, they each represent portions of the reference genome that are equal sizes. In alternative embodiments, each of the bins have independent sizes. That is, they each represent portions of the reference genome that are the same or different sizes. In some embodiments, a plurality of bins representing a human reference genome includes at least 23 bins, at least 50 bins, at least 100 bins, at least 1000 bins, at least 5000 bins, at least 10,000 bins, at least 50,000 bins, at least 100,000 bins or more. In some embodiments, a bin represents at least 50 bp, at least 100 bp, at least 500 bp, at least 1000 by, at least 25000 bp, at least 5000 bp, at least 10,000 bp, at least 50,000 bp, at least 100,000 bp, at least 250,000 bp, at least 500,000 bp, at least 1 MB, at least 2.5 MB, at least 5 MB, at least 10 MB, at least 25 MB, or more of a genome for the species of a subject, e.g., a human. In some embodiments, a bin represents no more than 50 MB, no more than 25 MB, no more than 10 MB, no more than 5 MB, no more than 2.5 MB, no more than 1 MB, no more than 0.5 MB, no more than 0.1 MB, no more than 50,000 bp, no more than 25,000 bp, no more than 10,000 bp, or less of a genome for the species of a subject, e.g., a human.

As used herein, a “bin-level” metric refers to a value representative of a plurality of characteristic values (e.g., methylation characteristics, fragment length characteristics, etc.) for nucleic acid sequences assigned to a respective bin.

As used herein, a “fragment-level” metric refers to a value for a characteristic of a unique sequence read; that is, a de-duplicated sequence read corresponding to a single nucleic acid fragment sequenced in a nucleic acid sequencing reaction.

In some embodiments, the human genome is divided into between 50 and 10,000 bins, with each bin representing an independent portion of the reference genome. Then, each respective bin is associated with a count of the variants mapping to the portion of reference genome the respective bin represents.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some instances, a parameter is used to increase or decrease the influence of a node (e.g., of a neural network), where the node comprises one or more activation functions. Assignment of parameters to specific inputs, outputs, and/or functions is not limited to any one paradigm for a given algorithm, model, regressor, and/or classifier but can be used in any suitable an algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods, as described elsewhere herein).

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As used herein, the term “classification” can refer to any number(s) or other characters(s) that are associated with a particular property of a sample. For example, in some embodiments, the term “classification” can refer to a type of cancer in a subject, a stage of cancer in a subject, a prognosis for a cancer in a subject, a tumor load, a presence of tumor metastasis in a subject, and the like. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the term “untrained classifier” refers to a classifier that has not been trained on a training dataset.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, an “actionable genomic alteration” or “actionable variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to be associated with a therapeutic course of action that is more likely to produce a positive effect in a cancer patient that has the actionable variant than in a similarly situated cancer patient that does not have the actionable variant. For instance, administration of EGFR inhibitors (e.g., afatinib, erlotinib, gefitinib) is more effective for treating non-small cell lung cancer in patients with an EGFR mutation in exons 19/21 than for treating non-small cell lung cancer in patients that do not have an EGFR mutations in exons 19/21. Accordingly, an EGFR mutation in exon 19/21 is an actionable variant. In some instances, an actionable variant is only associated with an improved treatment outcome in one or a group of specific cancer types. In other instances, an actionable variant is associated with an improved treatment outcome in substantially all cancer types.

As used herein, a “variant of uncertain significance” or “VUS” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), whose impact on disease development/progression is unknown.

As used herein, a “benign variant” or “likely benign variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to not contribute to disease development/progression.

As used herein, a “pathogenic variant” or “likely pathogenic variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to contribute to disease development/progression.

As used herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the therapeutic agent being administered.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

Reference will now be made in detail to embodiments, examples of which are illustrated in the accompanying drawings. In the following detailed description, numerous specific details are set forth in order to provide a thorough understanding of the present disclosure, including example systems, methods, techniques, instruction sequences, and computing machine program products that embody illustrative implementations. However, the illustrative discussions below are not intended to be exhaustive or to limit the implementations to the precise forms disclosed. Many modifications and variations are possible in view of the above teachings. The features described herein are not limited by the illustrated ordering of acts or events, as some acts can occur in different orders and/or concurrently with other acts or events.

The implementations provided herein are chosen and described in order to best explain the principles and their practical applications, to thereby enable others skilled in the art to best utilize the various embodiments with various modifications as are suited to the particular use contemplated. In some instances, well-known methods, procedures, components, circuits, and networks have not been described in detail so as not to unnecessarily obscure aspects of the embodiments. In other instances, it will be apparent to one of ordinary skill in the art that the present disclosure may be practiced without one or more of the specific details.

It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that though such a design effort might be complex and time-consuming, it will nevertheless be a routine undertaking of engineering for those of ordering skill in the art having the benefit of the present disclosure.

### Example System Embodiments

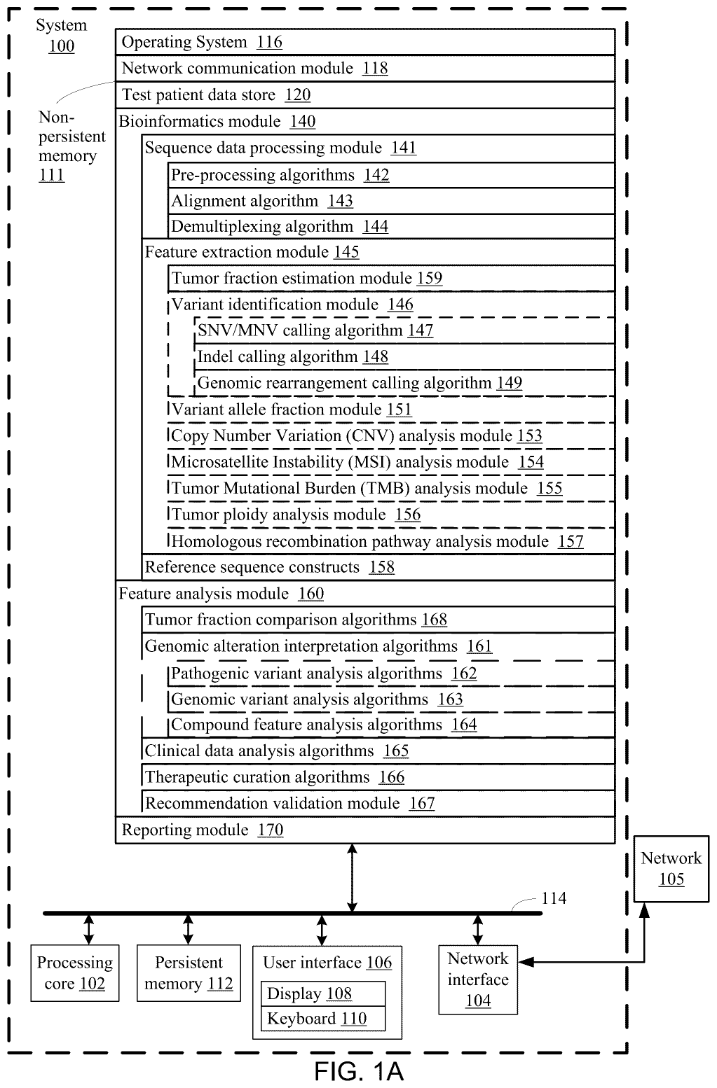

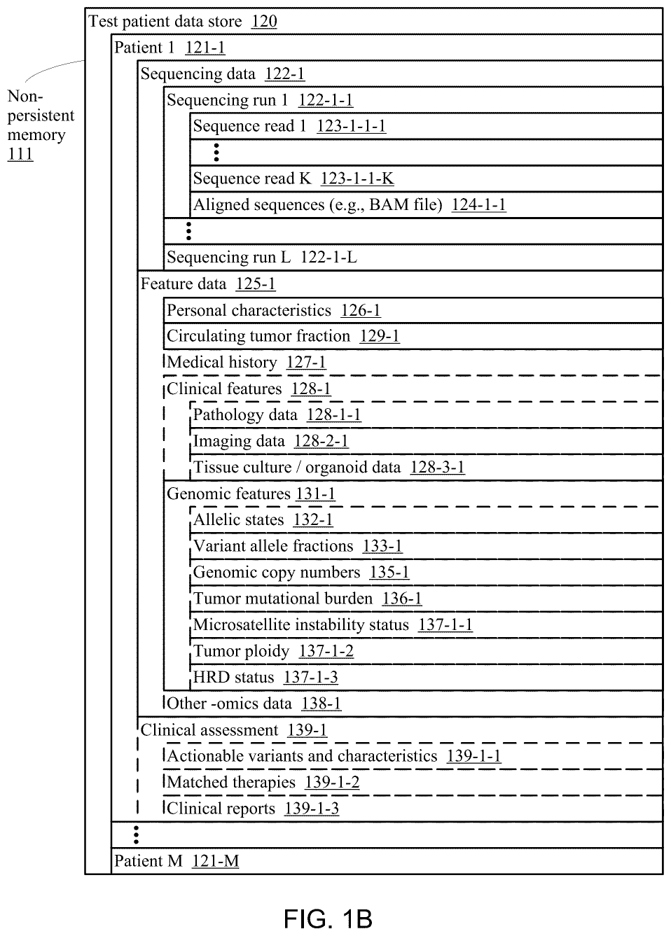

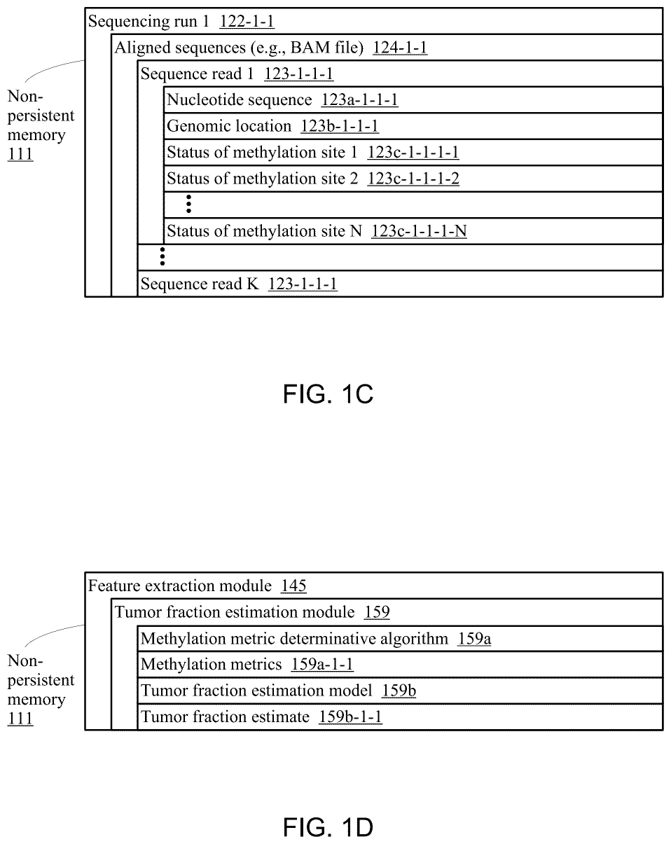

Now that an overview of some aspects of the present disclosure and some definitions used in the present disclosure have been provided, details of an exemplary system for providing clinical support for personalized cancer therapy using a liquid biopsy assay are now described in conjunction with FIGS. 1A-ID. FIGS. 1A-1D collectively illustrate the topology of an example system for providing clinical support for personalized cancer therapy using a liquid biopsy assay, in accordance with some embodiments of the present disclosure. Advantageously, the example system illustrated in FIGS. 1A-1D improves upon conventional methods for providing clinical support for personalized cancer therapy by providing methods of early cancer detection and circulating tumor fraction estimation.

FIG. 1A is a block diagram illustrating a system in accordance with some implementations. The device 100 in some implementations includes one or more processing units CPU(s) 102 (also referred to as processors), one or more network interfaces 104, a user interface 106, e.g., including a display 108 and/or an input 110 (e.g., a mouse, touchpad, keyboard, etc.), a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an operating system 116, which includes procedures for handling various basic system services and for performing hardware dependent tasks;
a network communication module (or instructions) 118 for connecting the system 100 with other devices and/or a communication network 105;
a test patient data store 120 for storing one or more collections of features from patients (e.g., subjects);
a bioinformatics module 140 for processing sequencing data and extracting features from sequencing data, e.g., from liquid biopsy sequencing assays;
a feature analysis module 160 for evaluating patient features, e.g., genomic alterations, compound genomic features, and clinical features; and
a reporting module 180 for generating and transmitting reports that provide clinical support for personalized cancer therapy.

Although FIGS. 1A-1D depict a “system 100,” the figures are intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112. For example, in various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations.

In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of system 100, that is addressable by system 100 so that system 100 may retrieve all or a portion of such data when needed.

For purposes of illustration in FIG. 1A, system 100 is represented as a single computer that includes all of the functionality for providing clinical support for personalized cancer therapy. However, while a single machine is illustrated, the term “system” shall also be taken to include any collection of machines that individually or jointly execute a set (or multiple sets) of instructions to perform any one or more of the methodologies discussed herein.

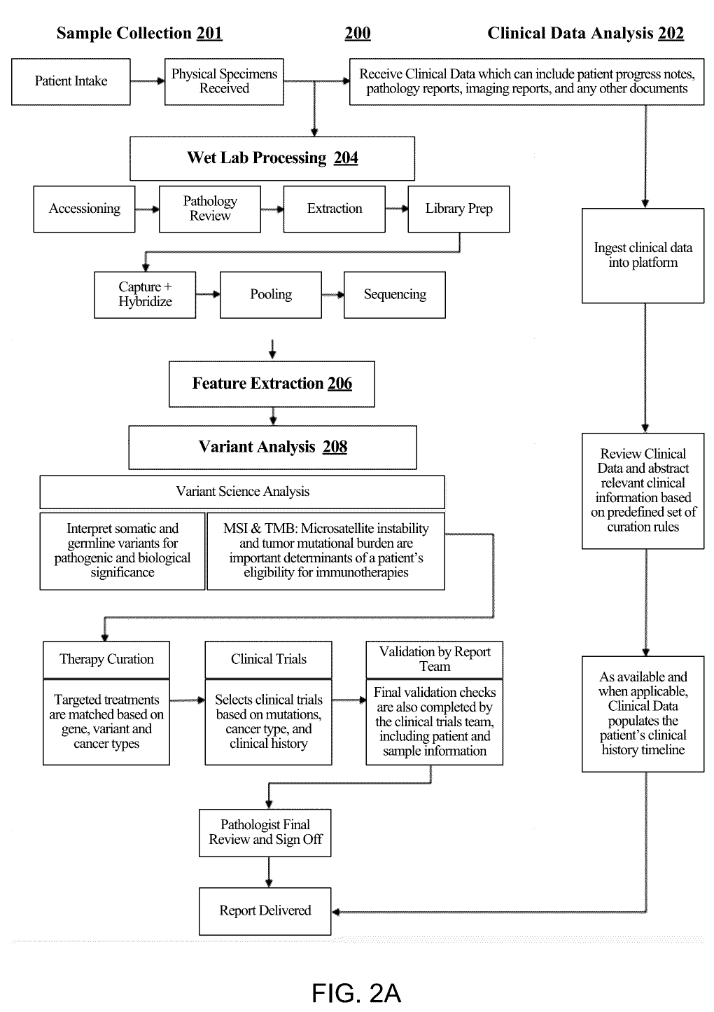

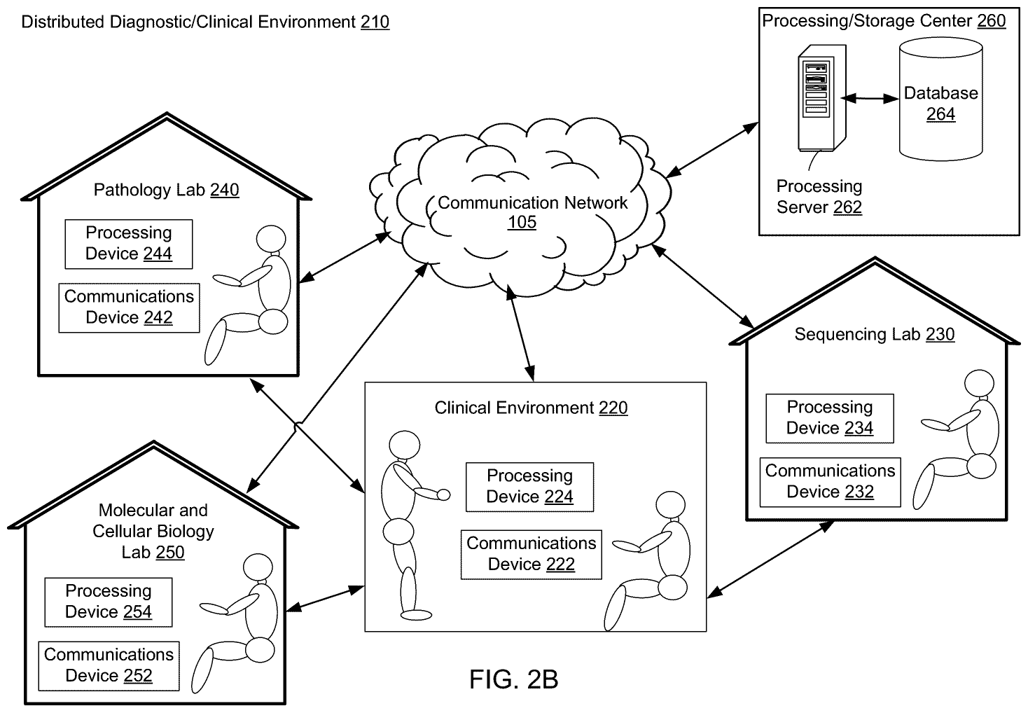

For example, in some embodiments, system 100 includes one or more computers. In some embodiments, the functionality for providing clinical support for personalized cancer therapy is spread across any number of networked computers and/or resides on each of several networked computers and/or is hosted on one or more virtual machines at a remote location accessible across the communications network 105. For example, different portions of the various modules and data stores illustrated in FIGS. 1A-1D can be stored and/or executed on the various instances of a processing device and/or processing server/database in the distributed diagnostic environment 210 illustrated in FIG. 2B (e.g., processing devices 224, 234, 244, and 254, processing server 262, and database 264).

The system may operate in the capacity of a server or a client machine in client-server network environment, as a peer machine in a peer-to-peer (or distributed) network environment, or as a server or a client machine in a cloud computing infrastructure or environment. The system may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine.

In another implementation, the system comprises a virtual machine that includes a module for executing instructions for performing any one or more of the methodologies disclosed herein. In computing, a virtual machine (VM) is an emulation of a computer system that is based on computer architectures and provides functionality of a physical computer. Some such implementations may involve specialized hardware, software, or a combination of hardware and software.

One of skill in the art will appreciate that any of a wide array of different computer topologies are used for the application and all such topologies are within the scope of the present disclosure.

### Test Patient Data Store (120)

Referring to FIG. 1B, in some embodiments, the system (e.g., system 100) includes a patient data store 120 that stores data for patients 121-1 to 121-M (e.g., cancer patients or patients being tested for cancer) including one or more sequencing data 122, feature data 125, and clinical assessments 139. These data are used and/or generated by the various processes stored in the bioinformatics module 140 and feature analysis module 160 of system 100, to ultimately generate a report providing clinical support for personalized cancer therapy of a patient. While the feature scope of patient data 121 across all patients may be informationally dense, an individual patient's feature set may be sparsely populated across the entirety of the collective feature scope of all features across all patients. That is to say, the data stored for one patient may include a different set of features that the data stored for another patient. Further, while illustrated as a single data construct in FIG. 1B, different sets of patient data may be stored in different databases or modules spread across one or more system memories.

In some embodiments, sequencing data 122 from one or more sequencing reactions 122-i, including a plurality of sequence reads 123-i-1 to 123-i-K, is stored in the test patient data store 120. The data store may include different sets of sequencing data from a single subject, corresponding to different samples from the patient, e.g., a tumor sample, liquid biopsy sample, tumor organoid derived from a patient tumor, and/or a normal sample, and/or to samples acquired at different times, e.g., while monitoring the progression, regression, remission, and/or recurrence of a cancer in a subject. The sequence reads may be in any suitable file format, e.g., BCL, FASTA, FASTQ, etc. In some embodiments, sequencing data 122 is accessed by a sequencing data processing module 141, which performs various pre-processing, genome alignment, and demultiplexing operations, as described in detail below with reference to bioinformatics module 140. In some embodiments, sequence data that has been aligned to a reference construct, e.g., BAM file 124, is stored in test patient data store 120.

In some embodiments, the test patient data store 120 includes feature data 125, e.g., that is useful for identifying clinical support for personalized cancer therapy. In some embodiments, the feature data 125 includes personal characteristics 126 of the patient, such as patient name, date of birth, gender, ethnicity, physical address, smoking status, alcohol consumption characteristic, anthropomorphic data, etc.

In some embodiments, the feature data 125 includes medical history data 127 for the patient, such as cancer diagnosis information (e.g., date of initial diagnosis, date of metastatic diagnosis, cancer staging, tumor characterization, tissue of origin, previous treatments and outcomes, adverse effects of therapy, therapy group history, clinical trial history, previous and current medications, surgical history, etc.), previous or current symptoms, previous or current therapies, previous treatment outcomes, previous disease diagnoses, diabetes status, diagnoses of depression, diagnoses of other physical or mental maladies, and family medical history. In some embodiments, the feature data 125 includes clinical features 128, such as pathology data 128-1, medical imaging data 128-2, and tissue culture and/or tissue organoid culture data 128-3.

In some embodiments, yet other clinical features, such as previous laboratory testing results, are stored in the test patient data store 120. Medical history data 127 and clinical features may be collected from various sources, including at intake directly from the patient, from an electronic medical record (EMR) or electronic health record (EHR) for the patient, or curated from other sources, such as fields from various testing records (e.g., genetic sequencing reports).

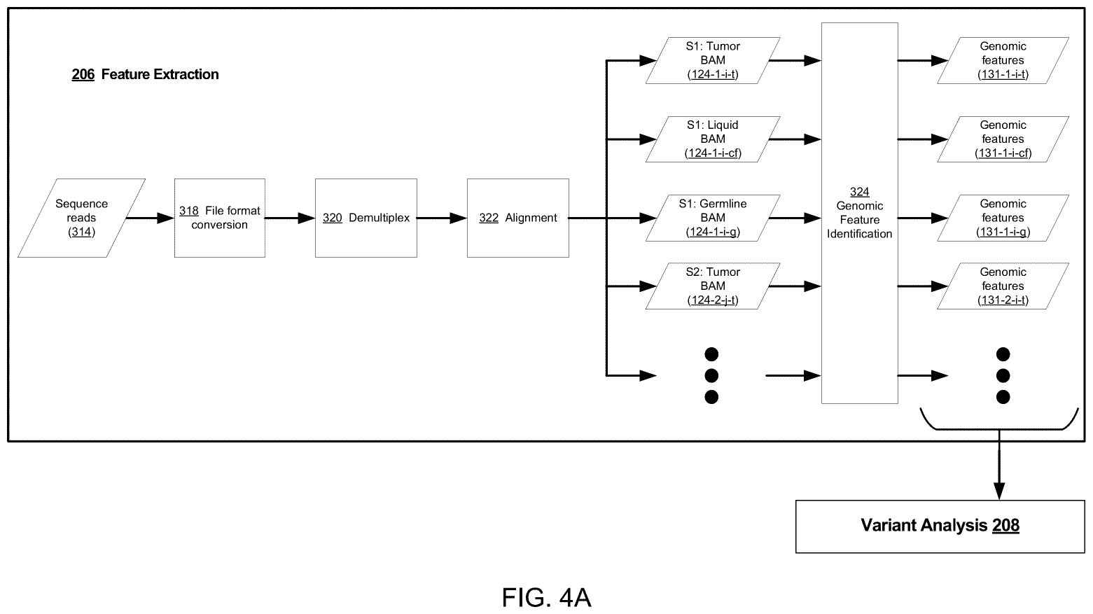

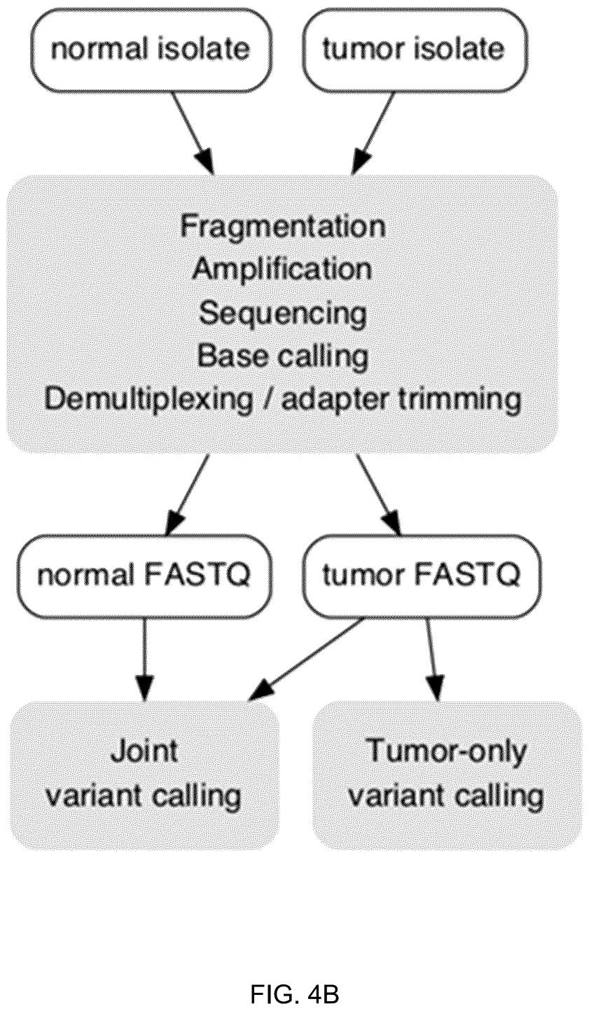

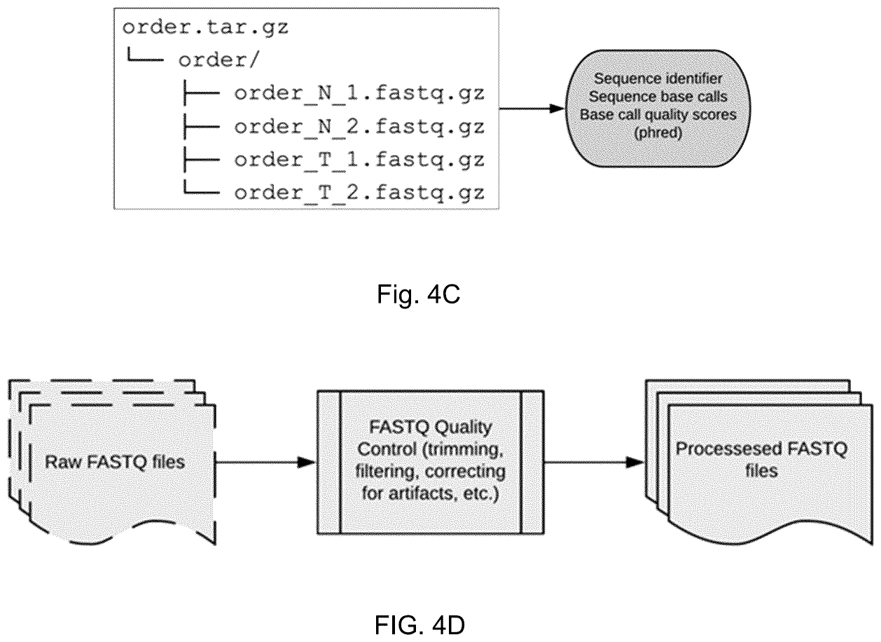

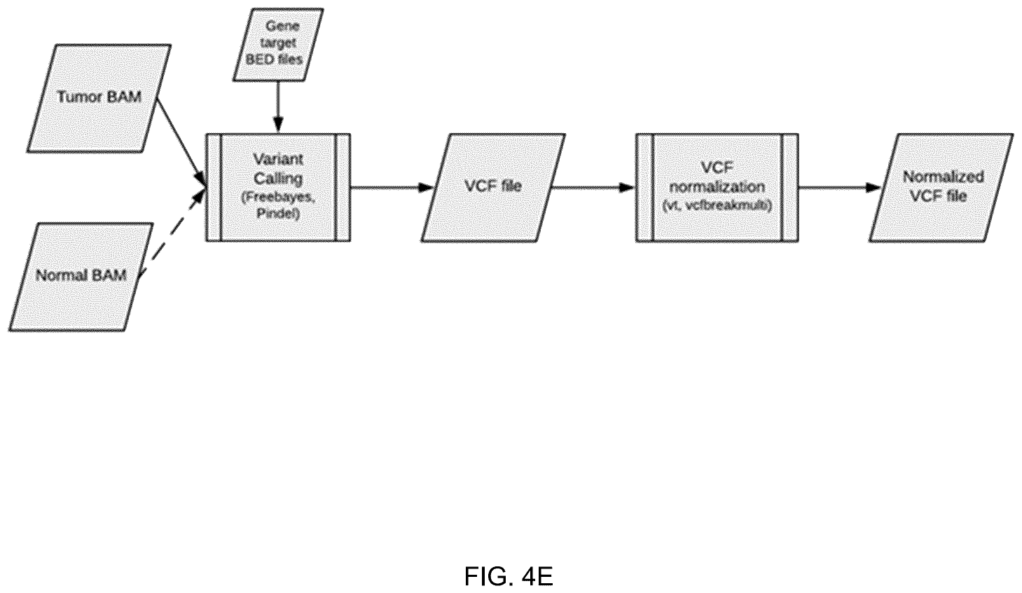

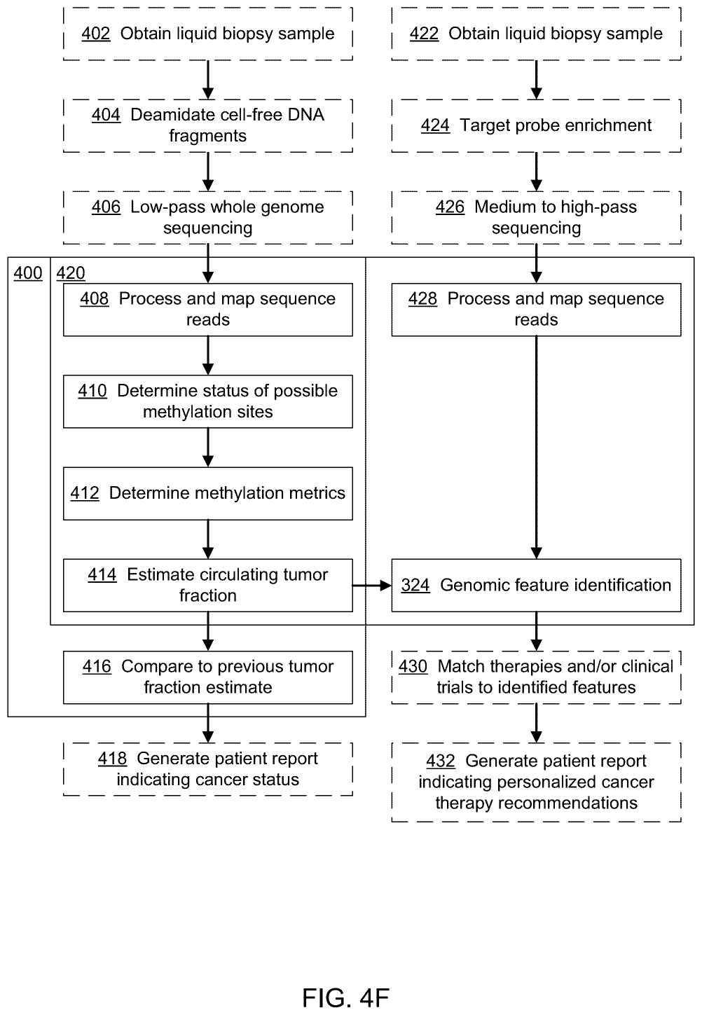

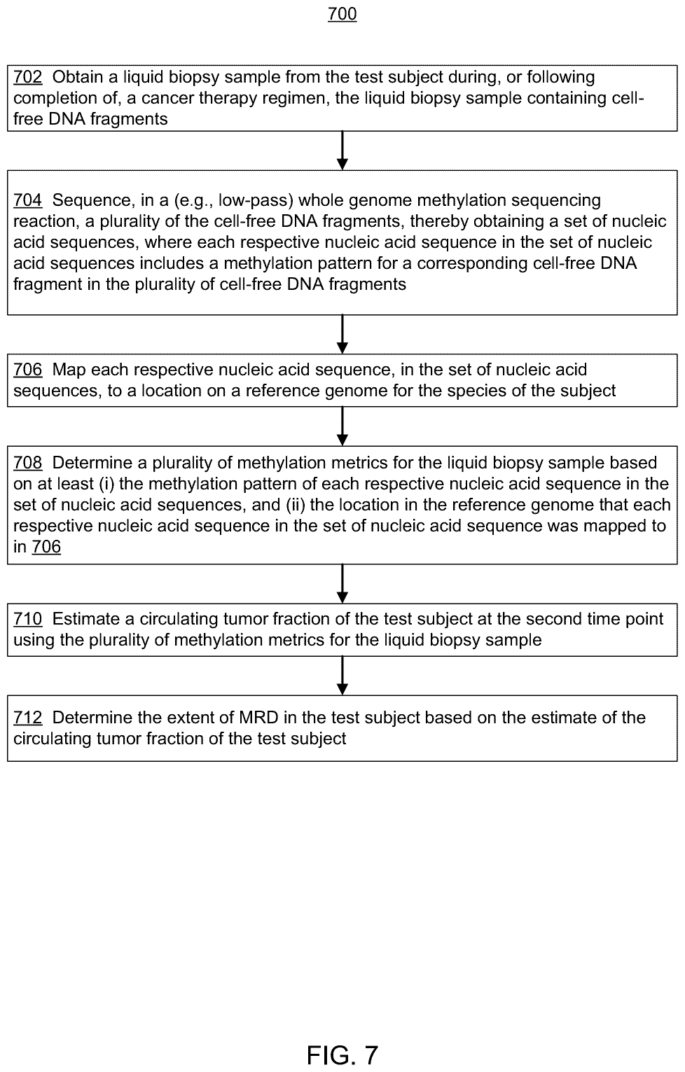

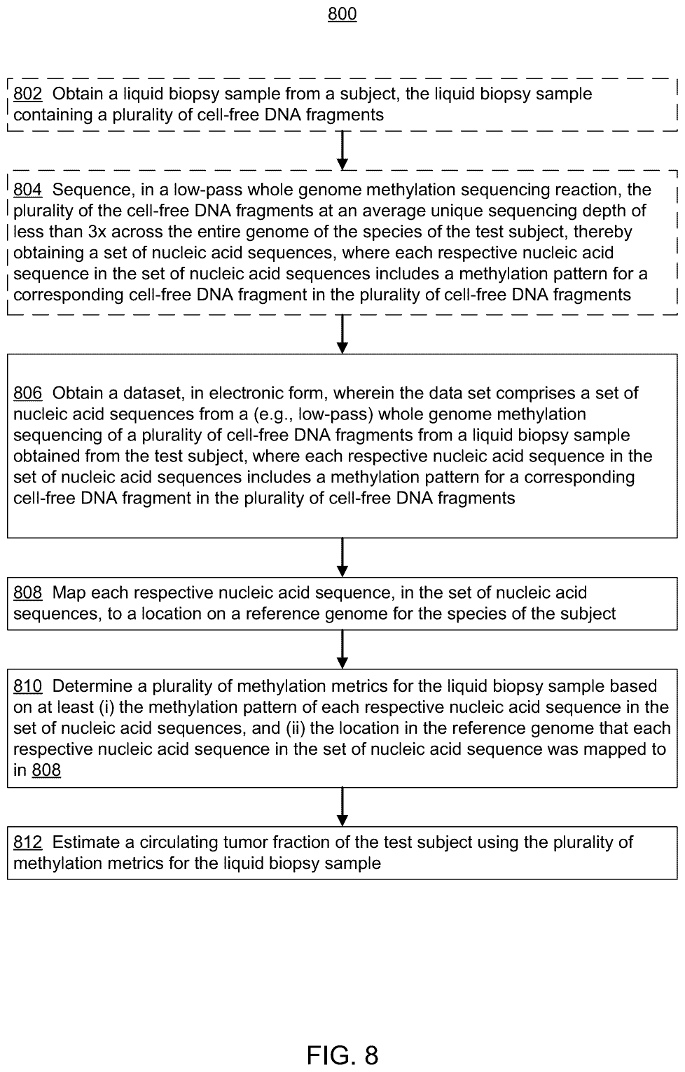

In some embodiments, the feature data 125 includes genomic features 131 for the patient. Non-limiting examples of genomic features include allelic states 132 (e.g., the identity of alleles at one or more loci, support for wild type or variant alleles at one or more loci, support for SNVs/MNVs at one or more loci, support for indels at one or more loci, and/or support for gene rearrangements at one or more loci), allelic fractions 133 (e.g., ratios of variant to reference alleles (or vice versa), methylation states 132 (e.g., a distribution of methylation patterns at one or more loci and/or support for aberrant methylation patterns at one or more loci), genomic copy numbers 135 (e.g., a copy number value at one or more loci and/or support for an aberrant (increased or decreased) copy number at one or more loci), tumor mutational burden 136 (e.g., a measure of the number of mutations in the cancer genome of the subject), and microsatellite instability status 137 (e.g., a measure of the repeated unit length at one or more microsatellite loci and/or a classification of the MSI status for the patient's cancer). In some embodiments, one or more of the genomic features 131 are determined by a nucleic acid bioinformatics pipeline, e.g., as described in detail below with reference to FIG. 4. In particular, in some embodiments, the feature data 125 include sequence data 122 from a whole genome methylation sequencing reaction (e.g., a low-pass whole genome methylation sequencing reaction). In some embodiments, aligned sequence files 124, e.g., BAM files, contain information extracted from sequence reads of unique DNA fragments, such as the nucleotide sequence 123a of the fragment, the genomic location 123b to which the DNA fragment maps, and the methylation status 123c of a plurality of possible methylation sites, e.g., CpG dinucleotides, as determined using the improved methods for using whole genome methylation sequencing reaction (e.g., a low-pass whole genome methylation sequencing reaction), as described in further detail below with reference to FIGS. 1C, 1D, 4F, and 5-9. In some embodiments, one or more of the genomic features 131 are obtained from an external testing source, e.g., not connected to the bioinformatics pipeline as described below.

In some embodiments, the feature data 125 further includes data 138 from other-omics fields of study. Non-limiting examples of -omics fields of study that may yield feature data useful for providing clinical support for personalized cancer therapy include transcriptomics, epigenomics, proteomics, metabolomics, metabonomics, microbiomics, lipodomics, glycomics, cellomics, and organoidomics.

In some embodiments, yet other features may include features derived from machine learning approaches, e.g., based at least in part on evaluation of any relevant molecular or clinical features, considered alone or in combination, not limited to those listed above. For instance, in some embodiments, one or more latent features learned from evaluation of cancer patient training datasets improve the diagnostic and prognostic power of the various analysis algorithms in the feature analysis module 160.

The skilled artisan will know of other types of features useful for providing clinical support for personalized cancer therapy. The listing of features above is merely representative and should not be construed to be limiting.

In some embodiments, a test patient data store 120 includes clinical assessment data 139 for patients, e.g., based off the feature data 125 collected for the subject. In some embodiments, the clinical assessment data 139 includes a catalogue of actionable variants and characteristics 139-1 (e.g., genomic alterations and compound metrics based on genomic features known or believed to be targetable by one or more specific cancer therapies), matched therapies 139-2 (e.g., the therapies known or believed to be particularly beneficial for treatment of subjects having actionable variants), and/or clinical reports 139-3 generated for the subject, e.g., based on identified actionable variants and characteristics 139-1 and/or matched therapies 139-2.

In some embodiments, clinical assessment data 139 is generated by analysis of feature data 125 using the various algorithms of feature analysis module 160, as described in further detail below. In some embodiments, clinical assessment data 139 is generated, modified, and/or validated by evaluation of feature data 125 by a clinician, e.g., an oncologist. For instance, in some embodiments, a clinician (e.g., at clinical environment 220) uses feature analysis module 160, or accesses test patient data store 120 directly, to evaluate feature data 125 to make recommendations for personalized cancer treatment of a patient. Similarly, in some embodiments, a clinician (e.g., at clinical environment 220) reviews recommendations determined using feature analysis module 160 and approves, rejects, or modifies the recommendations, e.g., prior to the recommendations being sent to a medical professional treating the cancer patient.

### Bioinformatics Module (140)

Referring again to FIG. 1A, the system (e.g., system 100) includes a bioinformatics module 140 that includes a feature extraction module 145 and optional ancillary data processing constructs, such as a sequence data processing module 141 and/or one or more reference sequence constructs 158 (e.g., a reference genome, exome, or targeted-panel construct that includes reference sequences for a plurality of loci targeted by a sequencing panel).

In some embodiments, bioinformatics module 140 includes a sequence data processing module 141 that includes instructions for processing sequence reads, e.g., raw sequence reads 123 from one or more sequencing reactions 122, prior to analysis by the various feature extraction algorithms, as described in detail below. In some embodiments, sequence data processing module 141 includes one or more pre-processing algorithms 142 that prepare the data for analysis. In some embodiments, the pre-processing algorithms 142 include instructions for converting the file format of the sequence reads from the output of the sequencer (e.g., a BCL file format) into a file format compatible with downstream analysis of the sequences (e.g., a FASTQ or FASTA file format). In some embodiments, the pre-processing algorithms 142 include instructions for evaluating the quality of the sequence reads (e.g., by interrogating quality metrics like Phred score, base-calling error probabilities, Quality (Q) scores, and the like) and/or removing sequence reads that do not satisfy a threshold quality (e.g., an inferred base call accuracy of at least 80%, at least 90%, at least 95%, at least 99%, at least 99.5%, at least 99.9%, or higher). In some embodiments, the pre-processing algorithms 142 include instructions for filtering the sequence reads for one or more properties, e.g., removing sequences failing to satisfy a lower or upper size threshold or removing duplicate sequence reads.

In some embodiments, sequence data processing module 141 includes one or more alignment algorithms 143, for aligning pre-processed sequence reads 123 to a reference sequence construct 158, e.g., a reference genome, exome, or targeted-panel construct. Many algorithms for aligning sequencing data to a reference construct are known in the art, for example, BWA, Blat, SHRIMP, LastZ, and MAQ. One example of a sequence read alignment package is the Burrows-Wheeler Alignment tool (BWA), which uses a Burrows-Wheeler Transform (BWT) to align short sequence reads against a large reference construct, allowing for mismatches and gaps. Li and Durbin, Bioinformatics, 25(14):1754-60 (2009), the content of which is incorporated herein by reference, in its entirety, for all purposes. Sequence read alignment packages import raw or pre-processed sequence reads 122, e.g., in BCL, FASTA, or FASTQ file formats, and output aligned sequence reads 124, e.g., in SAM or BAM file formats.

In some embodiments, sequence data processing module 141 includes one or more demultiplexing algorithms 144, for dividing sequence read or sequence alignment files generated from sequencing reactions of pooled nucleic acids into separate sequence read or sequence alignment files, each of which corresponds to a different source of nucleic acids in the nucleic acid sequencing pool. For instance, because of the cost of sequencing reactions, it is common practice to pool nucleic acids from a plurality of samples into a single sequencing reaction. The nucleic acids from each sample are tagged with a sample-specific and/or molecule-specific sequence tag (e.g., a UMI), which is sequenced along with the molecule. In some embodiments, demultiplexing algorithms 144 sort these sequence tags in the sequence read or sequence alignment files to demultiplex the sequencing data into separate files for each of the samples included in the sequencing reaction.

Bioinformatics module 140 includes a feature extraction module 145, which includes instructions for identifying diagnostic features, e.g., genomic features 131, from sequencing data 122 of biological samples from a subject, e.g., one or more of a solid tumor sample, a liquid biopsy sample, or a normal tissue (e.g., control) sample. For instance, in some embodiments, a feature extraction algorithm compares the identity of one or more nucleotides at a locus from the sequencing data 122 to the identity of the nucleotides at that locus in a reference sequence construct (e.g., a reference genome, exome, or targeted-panel construct) to determine whether the subject has a variant at that locus. In some embodiments, a feature extraction algorithm evaluates data other than the raw sequence, to identify a genomic alteration in the subject, e.g., an allelic ratio, a relative copy number, a repeat unit distribution, etc.

For instance, in some embodiments, feature extraction module 145 includes one or more variant identification modules that include instructions for various variant calling processes. In some embodiments, variants in the germline of the subject are identified, e.g., using a germline variant identification module 146. In some embodiments, variants in the cancer genome, e.g., somatic variants, are identified, e.g., using a somatic variant identification module 150. While separate germline and somatic variant identification modules are illustrated in FIG. 1A, in some embodiments they are integrated into a single module. In some embodiments, the variant identification module includes instructions for identifying one or more of nucleotide variants (e.g., single nucleotide variants (SNV) and multi-nucleotide variants (MNV)) using one or more SNV/MNV calling algorithms (e.g., algorithms 147 and/or 151), indels (e.g., insertions or deletions of nucleotides) using one or more indel calling algorithms (e.g., algorithms 148 and/or 152), and genomic rearrangements (e.g., inversions, translocation, and fusions of nucleotide sequences) using one or more genomic rearrangement calling algorithms (e.g., algorithms 149 and/or 153).

A SNV/MNV algorithm 147 may identify a substitution of a single nucleotide that occurs at a specific position in the genome. For example, at a specific base position, or locus, in the human genome, the C nucleotide may appear in most individuals, but in a minority of individuals, the position is occupied by an A. This means that there is a SNP at this specific position and the two possible nucleotide variations, C or A, are said to be alleles for this position. SNPs underlie differences in human susceptibility to a wide range of diseases (e.g.—sickle-cell anemia, β-thalassemia and cystic fibrosis result from SNPs). The severity of illness and the way the body responds to treatments are also manifestations of genetic variations. For example, a single-base mutation in the APOE (apolipoprotein E) gene is associated with a lower risk for Alzheimer's disease. A single-nucleotide variant (SNV) is a variation in a single nucleotide without any limitations of frequency and may arise in somatic cells. A somatic single-nucleotide variation (e.g., caused by cancer) may also be called a single-nucleotide alteration. An MNP (Multiple-nucleotide polymorphisms) module may identify the substitution of consecutive nucleotides at a specific position in the genome.

An indel calling algorithm 148 may identify an insertion or deletion of bases in the genome of an organism classified among small genetic variations. While indels usually measure from 1 to 10 000 base pairs in length, a microindel is defined as an indel that results in a net change of 1 to 50 nucleotides. Indels can be contrasted with a SNP or point mutation. An indel inserts and/or deletes nucleotides from a sequence, while a point mutation is a form of substitution that replaces one of the nucleotides without changing the overall number in the DNA. Indels, being insertions and/or deletions, can be used as genetic markers in natural populations, especially in phylogenetic studies. Indel frequency tends to be markedly lower than that of single nucleotide polymorphisms (SNP), except near highly repetitive regions, including homopolymers and microsatellites.

A genomic rearrangement algorithm 149 may identify hybrid genes formed from two previously separate genes. It can occur as a result of: translocation, interstitial deletion, or chromosomal inversion. Gene fusion can play an important role in tumorigenesis. Fusion genes can contribute to tumor formation because fusion genes can produce much more active abnormal protein than non-fusion genes. Often, fusion genes are oncogenes that cause cancer; these include BCR-ABL, TEL-AML1 (ALL with t(12; 21)), AML1-ETO (M2 AML with t(8; 21)), and TMPRSS2-ERG with an interstitial deletion on chromosome 21, often occurring in prostate cancer. In the case of TMPRSS2-ERG, by disrupting androgen receptor (AR) signaling and inhibiting AR expression by oncogenic ETS transcription factor, the fusion product regulates prostate cancer. Most fusion genes are found from hematological cancers, sarcomas, and prostate cancer. BCAM-AKT2 is a fusion gene that is specific and unique to high-grade serous ovarian cancer. Oncogenic fusion genes may lead to a gene product with a new or different function from the two fusion partners. Alternatively, a proto-oncogene is fused to a strong promoter, and thereby the oncogenic function is set to function by an upregulation caused by the strong promoter of the upstream fusion partner. The latter is common in lymphomas, where oncogenes are juxtaposed to the promoters of the immunoglobulin genes. Oncogenic fusion transcripts may also be caused by trans-splicing or read-through events. Since chromosomal translocations play such a significant role in neoplasia, a specialized database of chromosomal aberrations and gene fusions in cancer has been created. This database is called Mitelman Database of Chromosome Aberrations and Gene Fusions in Cancer.

In some embodiments, feature extraction module 145 includes instructions for identifying one or more complex genomic alterations (e.g., features that incorporate more than a change in the primary sequence of the genome) in the cancer genome of the subject. For instance, in some embodiments, feature extraction module 145 includes modules for identifying one or more of copy number variation (e.g., copy number variation analysis module 153), microsatellite instability status (e.g., microsatellite instability analysis module 154), tumor mutational burden (e.g., tumor mutational burden analysis module 155), tumor ploidy (e.g., tumor ploidy analysis module 156), and homologous recombination pathway deficiencies (e.g., homologous recombination pathway analysis module 157).

### Feature Analysis Module (160)

Referring again to FIG. 1A, the system (e.g., system 100) includes a feature analysis module 160 that includes one or more genomic alteration interpretation algorithms 161, one or more optional clinical data analysis algorithms 165, an optional therapeutic curation algorithm 165, and an optional recommendation validation module 167. In some embodiments, feature analysis module 160 identifies actionable variants and characteristics 139-1 and corresponding matched therapies 139-2 and/or clinical trials using one or more analysis algorithms (e.g., algorithms 162, 163, 164, and 165) to evaluate feature data 125. The identified actionable variants and characteristics 139-1 and corresponding matched therapies 139-2, which are optionally stored in test patient data store 120, are then curated by feature analysis module 160 to generate a clinical report 139-3, which is optionally validated by a user, e.g., a clinician, before being transmitted to a medical professional, e.g., an oncologist, treating the patient.

In some embodiments, the genomic alteration interpretation algorithms 161 include instructions for evaluating the effect that one or more genomic features 131 of the subject, e.g., as identified by feature extraction module 145, have on the characteristics of the patient's cancer and/or whether one or more targeted cancer therapies may improve the clinical outcome for the patient. For example, in some embodiments, one or more genomic variant analysis algorithms 163 evaluate various genomic features 131 by querying a database, e.g., a look-up-table (“LUT”) of actionable genomic alterations, targeted therapies associated with the actionable genomic alterations, and any other conditions that should be met before administering the targeted therapy to a subject having the actionable genomic alteration. For instance, evidence suggests that depatuxizumab mafodotin (an anti-EGFR mAb conjugated to monomchyl auristatin F) has improved efficacy for the treatment of recurrent glioblastomas having EGFR focal amplifications. van den Bent M. et al., Cancer Chemother Pharmacol., 80(6):1209-17 (2017). Accordingly, the actionable genomic alteration LUT would have an entry for the focal amplification of the EGFR gene indicating that depatuxizumab mafodotin is a targeted therapy for glioblastomas (e.g., recurrent glioblastomas) having a focal gene amplification. In some instances, the LUT may also include counter indications for the associated targeted therapy, e.g., adverse drug interactions or personal characteristics that are counter-indicated for administration of the particular targeted therapy.

In some embodiments, a genomic alteration interpretation algorithm 161 determines whether a particular genomic feature 131 should be reported to a medical professional treating the cancer patient. In some embodiments, genomic features 131 (e.g., genomic alterations and compound features) are reported when there is clinical evidence that the feature significantly impacts the biology of the cancer, impacts the prognosis for the cancer, and/or impacts pharmacogenomics, e.g., by indicating or counter-indicating particular therapeutic approaches. For instance, a genomic alteration interpretation algorithm 161 may classify a particular CNV feature 135 as “Reportable,” e.g., meaning that the CNV has been identified as influencing the character of the cancer, the overall disease state, and/or pharmacogenomics, as “Not Reportable,” e.g., meaning that the CNV has not been identified as influencing the character of the cancer, the overall disease state, and/or pharmacogenomics, as “No Evidence,” e.g., meaning that no evidence exists supporting that the CNV is “Reportable” or “Not Reportable,” or as “Conflicting Evidence,” e.g., meaning that evidence exists supporting both that the CNV is “Reportable” and that the CNV is “Not Reportable.”

In some embodiments, the genomic alteration interpretation algorithms 161 include one or more pathogenic variant analysis algorithms 162, which evaluate various genomic features to identify the presence of an oncogenic pathogen associated with the patient's cancer and/or targeted therapies associated with an oncogenic pathogen infection in the cancer. For instance, RNA expression patterns of some cancers are associated with the presence of an oncogenic pathogen that is helping to drive the cancer. See, for example, U.S. patent application Ser. No. 16/802,126, filed Feb. 26, 2020, the content of which is hereby incorporated by reference, in its entirety, for all purposes. In some instances, the recommended therapy for the cancer is different when the cancer is associated with the oncogenic pathogen infection than when it is not. Accordingly, in some embodiments, e.g., where feature data 125 includes RNA abundance data for the cancer of the patient, one or more pathogenic variant analysis algorithms 162 evaluate the RNA abundance data for the patient's cancer to determine whether a signature exists in the data that indicates the presence of the oncogenic pathogen in the cancer. Similarly, in some embodiments, bioinformatics module 140 includes an algorithm that searches for the presence of pathogenic nucleic acid sequences in sequencing data 122. See, for example, U.S. Provisional Patent Application Ser. No. 62/978,067, filed Feb. 18, 2020, the content of which is hereby incorporated by reference, in its entirety, for all purposes. Accordingly, in some embodiments, one or more pathogenic variant analysis algorithms 162 evaluates whether the presence of an oncogenic pathogen in a subject is associated with an actionable therapy for the infection. In some embodiments, system 100 queries a database, e.g., a look-up-table (“LUT”), of actionable oncogenic pathogen infections, targeted therapies associated with the actionable infections, and any other conditions that should be met before administering the targeted therapy to a subject that is infected with the oncogenic pathogen. In some instances, the LUT may also include counter indications for the associated targeted therapy, e.g., adverse drug interactions or personal characteristics that are counter-indicated for administration of the particular targeted therapy.

In some embodiments, the genomic alteration interpretation algorithms 161 include one or more multi-feature analysis algorithms 164 that evaluate a plurality of features to classify a cancer with respect to the effects of one or more targeted therapies. For instance, in some embodiments, feature analysis module 160 includes one or more classifiers trained against feature data, one or more clinical therapies, and their associated clinical outcomes for a plurality of training subjects to classify cancers based on their predicted clinical outcomes following one or more therapies.

In some embodiments, the classifier is implemented as an artificial intelligence engine and may include gradient boosting models, random forest models, neural networks (NN), regression models, Naive Bayes models, and/or machine learning algorithms (MLA). A MLA or a NN may be trained from a training data set that includes one or more features 125, including personal characteristics 126, medical history 127, clinical features 128, genomic features 131, and/or other -omic features 138. MLAs include supervised algorithms (such as algorithms where the features/classifications in the data set are annotated) using linear regression, logistic regression, decision trees, classification and regression trees, naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

NNs include conditional random fields, convolutional neural networks, attention based neural networks, deep learning, long short term memory networks, or other neural models where the training data set includes a plurality of tumor samples, RNA expression data for each sample, and pathology reports covering imaging data for each sample.

While MLA and neural networks identify distinct approaches to machine learning, the terms may be used interchangeably herein. Thus, a mention of MLA may include a corresponding NN or a mention of NN may include a corresponding MLA unless explicitly stated otherwise. Training may include providing optimized datasets, labeling these traits as they occur in patient records, and training the MLA to predict or classify based on new inputs. Artificial NNs are efficient computing models which have shown their strengths in solving hard problems in artificial intelligence. They have also been shown to be universal approximators, that is, they can represent a wide variety of functions when given appropriate parameters.

In some embodiments, system 100 includes a classifier training module that includes instructions for training one or more untrained or partially trained classifiers based on feature data from a training dataset. In some embodiments, system 100 also includes a database of training data for use in training the one or more classifiers. In other embodiments, the classifier training module accesses a remote storage device hosting training data. In some embodiments, the training data includes a set of training features, including but not limited to, various types of the feature data 125 illustrated in FIG. 1B. In some embodiments, the classifier training module uses patient data 121, e.g., when test patient data store 120 also stores a record of treatments administered to the patient and patient outcomes following therapy. Additional details relating to the training and implementation of multi-feature classifiers is provided below.

In some embodiments, feature analysis module 160 includes one or more clinical data analysis algorithms 165, which evaluate clinical features 128 of a cancer to identify targeted therapies which may benefit the subject. For example, in some embodiments, e.g., where feature data 125 includes pathology data 128-1, one or more clinical data analysis algorithms 165 evaluate the data to determine whether an actionable therapy is indicated based on the histopathology of a tumor biopsy from the subject, e.g., which is indicative of a particular cancer type and/or stage of cancer. In some embodiments, system 100 queries a database, e.g., a look-up-table (“LUT”), of actionable clinical features (e.g., pathology features), targeted therapies associated with the actionable features, and any other conditions that should be met before administering the targeted therapy to a subject associated with the actionable clinical features 128 (e.g., pathology features 128-1). In some embodiments, system 100 evaluates the clinical features 128 (e.g., pathology features 128-1) directly to determine whether the patient's cancer is sensitive to a particular therapeutic agent. Further details on example methods, systems, and algorithms for classifying cancer and identifying targeted therapies based on clinical data, such as pathology data 128-1, imaging data 138-2, and/or tissue culture/organoid data 128-3 are discussed, for example, in U.S. patent application Ser. No. 16/830,186, filed on Mar. 25, 2020, U.S. patent application Ser. No. 16/789,363, filed on Feb. 12, 2020, and U.S. Provisional Application No. 63/007,874, filed on Apr. 9, 2020, the contents of which are hereby incorporated by reference, in their entireties, for all purposes.

In some embodiments, feature analysis module 160 includes a clinical trials module that evaluates test patient data 121 to determine whether the patient is eligible for inclusion in a clinical trial for a cancer therapy, e.g., a clinical trial that is currently recruiting patients, a clinical trial that has not yet begun recruiting patients, and/or an ongoing clinical trial that may recruit additional patients in the future. In some embodiments, a clinical trial module evaluates test patient data 121 to determine whether the results of a clinical trial are relevant for the patient, e.g., the results of an ongoing clinical trial and/or the results of a completed clinical trial. For instance, in some embodiments, system 100 queries a database, e.g., a look-up-table (“LUT”) of clinical trials, e.g., active and/or completed clinical trials, and compares patient data 121 with inclusion criteria for the clinical trials, stored in the database, to identify clinical trials with inclusion criteria that closely match and/or exactly match the patient's data 121. In some embodiments, a record of matching clinical trials, e.g., those clinical trials that the patient may be eligible for and/or that may inform personalized treatment decisions for the patient, are stored in clinical assessment database 139.

In some embodiments, feature analysis module 160 includes a therapeutic curation algorithm 166 that assembles actionable variants and characteristics 139-1, matched therapies 139-2, and/or relevant clinical trials identified for the patient, as described above. In some embodiments, a therapeutic curation algorithm 166 evaluates certain criteria related to which actionable variants and characteristics 139-1, matched therapies 139-2, and/or relevant clinical trials should be reported and/or whether certain matched therapies, considered alone or in combination, may be counter-indicated for the patient, e.g., based on personal characteristics 126 of the patient and/or known drug-drug interactions. In some embodiments, the therapeutic curation algorithm then generates one or more clinical reports 139-3 for the patient. In some embodiments, the therapeutic curation algorithm generates a first clinical report 139-3-1 that is to be reported to a medical professional treating the patient and a second clinical report 139-3-2 that will not be communicated to the medical professional, but may be used to improve various algorithms within the system.

In some embodiments, feature analysis module 160 includes a recommendation validation module 167, that includes an interface allowing a clinician to review, modify, and approve a clinical report 139-3 prior to the report being sent to a medical professional, e.g., an oncologist, treating the patient.

In some embodiments, each of the one or more feature collections, sequencing modules, bioinformatics modules (including, e.g., alteration module(s), structural variant calling and data processing modules), classification modules and outcome modules are communicatively coupled to a data bus to transfer data between each module for processing and/or storage. In some alternative embodiments, each of the feature collection, alteration module(s), structural variant and feature store are communicatively coupled to each other for independent communication without sharing the data bus.

Further details on systems and exemplary embodiments of modules and feature collections are discussed in PCT Application PCT/US19/69149, titled “A METHOD AND PROCESS FOR PREDICTING AND ANALYZING PATIENT COHORT RESPONSE, PROGRESSION, AND SURVIVAL,” filed Dec. 31, 2019, which is hereby incorporated herein by reference in its entirety.

### Multi-Feature Classifiers and Machine Learning:

In some embodiments, the methods and systems described herein train and/or employ multi-feature classifiers (e.g., ensemble models and classifiers) and/or machine learning strategies to improve characterization of a patient's cancer and/or improve clinical outcomes by improving clinical support for personalized cancer therapies.

In some embodiments, one or more results obtained from a bioinformatics analysis pipeline for a respective biological sample (e.g., a liquid biopsy sample) are used as features to train a classifier, e.g., to improve methods of early cancer detection, circulating tumor fraction estimation, to classify a cancer condition of a patient, to identify personalized therapeutic strategies for a cancer patient, and/or to identify clinical trials relevant to a cancer patient. In some embodiments, a classifier is trained against data from a plurality of patients, where each respective patient in the plurality of patients has the same cancer condition (e.g., a presence or absence of cancer, a type of cancer, a stage of cancer, and/or a tissue-of-origin). In some alternative embodiments, a first one or more patients (or a first subset of patients) in the plurality of patients has a cancer condition that is different from a second one or more patients (or a second subset of patients) in the plurality of training patients. In some embodiments, to improve the training of the classifier, the classifier is trained against data from a plurality of patients, where one or more patients in the plurality of patients have two or more cancer conditions (e.g., 2, 3, 4, 5, 6, 7, 8, 9, 10, or more than 10 cancer conditions).

In some embodiments, features used to train a classifier include any of the features described above with respect to patient data store 120. For example, in some embodiments, a classifier is trained against the status of one or more variant alleles in the cancer of each training subject. In some such embodiments, the classifier is trained against the methylation status of nucleic acids, e.g., cfDNA from a liquid biopsy sample (e.g., a blood sample). In some embodiments, the features used to train the classifier include the methylation status of one or more genes listed in Table 1. In some embodiments, the classifier is trained against features of at least five of the genes listed in Table 1. In some embodiments, the classifier is trained against features of at least 10, at least 15, at least 20, at least 30, at least 40, at least 50, at least 60, at least 80, at least 100, or all of the genes listed in Table 1. In some embodiments, the classifier is trained against the methylation status of one or more genes not listed in Table 1. In some alternative embodiments, the classifier is trained against the methylation status of at least 5, at least 10, at least 15, at least 20, at least 30, at least 40, at least 50, at least 60, at least 80, or at least 100 genes not listed in Table 1.

In yet other embodiments, the classifier is trained against the methylation status of a large number of possible methylation sites across the entire genome, regardless of whether those sites fall within a gene sequence, a gene regulatory sequence (e.g., a promoter, enhancer, silencer, etc.), or merely in intra-genic regions for which no functionality has yet been elucidated. In this regard, in some embodiments, selection of particular features is performed stochastically, that is without biasing for a particular sequence context. In other embodiments, feature selection is performed around predetermined parameters, e.g., where the putative methylation site is within a particular type of sequence context, e.g., a gene, exon, intron, promoter region, enhancer region, silencer region, etc.

In some embodiments, methylation features are combined with other features derivable from whole genome methylation sequencing (e.g., low-pass whole genome methylation sequencing), low-pass whole genome sequencing, medium or high-pass whole genome sequencing, and/or target-panel sequencing reaction of a biological sample, e.g., a liquid biopsy sample.

In some embodiments, a methylation feature is combined with a sequence read-level feature, such as a genomic position of the sequence read, a length of a cell-free DNA fragment (e.g., producing a paired end read), the methylation pattern of any cytosine and/or any cytosine in a particular sequence context, the presence of a variant allele (e.g., a germline variant, a somatic variant, and/or a variant arising from clonal hematopoiesis), a sequence read quality score, and the like.

In some embodiments, a methylation feature is combined with a bin-level feature. For instance, in some embodiments, nucleic acid sequences determined from a methylation sequencing reaction (e.g., nucleic acid sequences representing unique cell-free DNA fragments in a liquid biopsy sample after collapsing redundant sequence reads, e.g., using UMI sequences and bagging methods as described herein) are binned according to a particular property of the nucleic acid sequence. For instance, in some embodiments, nucleic acid sequences are assigned to a respective bin, in a plurality of bins, according to the position within a reference sequence (e.g., the human genome) to which the sequence maps.

In some embodiments, the plurality of bins includes at least 10, 25, 50, 100, 250, 500, 1000, 2500, 5000, 10,000, 25,000, 50,000, 10,000, 250,000, 500,000, 1,00,000, 2,000,000, or more bins distributed across the reference sequence (e.g., the genome) for the species of the subject. In some embodiments, the bins are distributed relatively uniformly across the reference sequence, e.g., such that the each encompasses a similar number of bases, e.g., about 0.5 kb, 1 kb, 2 kb, 5 kb, 10 kb, 25 kb, 50 kb, 100 kb or more bases. Each respective bin in the plurality of bins represents a corresponding region of a reference sequence (e.g., genome) for the species of the subject. In some embodiments, the bins are distributed relatively uniformly across the reference sequence, e.g., such that the each encompasses a similar number of bases, e.g., about 0.5 kb, 1 kb, 2 kb, 5 kb, 10 kb, 25 kb, 50 kb, 100 kb or more bases. In some embodiments, the bin size is fixed, e.g., across the entire genome or across a particular chromosome. In other embodiments, the bin sizes are variable, e.g., according to some property of the genome of the species of the subject, e.g., the density of possible methylation sites.

In one embodiment, a binned methylation feature is used as an input (a feature) for a model (e.g., a component model) used by the methods and systems described herein. In some embodiments, determining the binned methylation feature includes binning nucleic acid sequences from a whole genome methylation sequencing reaction, e.g., a low-pass whole genome sequencing reaction), according to a fixed or variable binning pattern, as described herein, and determining a metric (e.g., a percentage, average, ratio relative to non-methylated sites, etc.) for the methylation pattern of one or more putative methylation sites (e.g., a cytosine residue, such as in a CpG dinucleotide) encompassed by the sequence reads assigned to a respective bin. When referring to binned methylation features—as opposed to fragment-level methylation features—the metric relates to one or more aggregate values for the methylation pattern determined across the nucleic acid sequences assigned to a respective bin, e.g., a distribution (or summary statistic thereof) of a methylation characteristic determined across the assigned sequence reads, a measure of central tendency for a methylation characteristic determined across the assigned sequence reads, etc. Such a bin-level feature can then be used as a feature in a model (e.g., a classifier or estimation model) trained to classify a cancer condition or provide an estimate of a circulating tumor fraction, according to the various embodiments described in the present disclosure.

In some embodiments, a bin-level methylation feature includes a metric for a methylation pattern at one or more putative methylation sites in the sequence reads assigned to a respective bin. For instance, one example of a bin-level methylation feature is a proportion of all putative methylation sites, present in the sequence reads assigned to a respective bin, that are methylated. Another example of a bin-level feature is a proportion of a subset of putative methylation sites (e.g., a subset of putative methylation sites that are differentially methylated in one or more types of cancerous tissue relative to a non-cancerous tissue or one or more different types of cancerous tissue), present in the sequence reads assigned to a respective bin, that are methylated. Another example of a bin-level feature is a measure of central tendency for a metric of the methylation patterns of respective nucleic acid sequences assigned to a respective bin (e.g., an average proportion of putative methylation sites, e.g., of all putative methylation sites or of a subset of putative methylation sites such as those that are differentially methylated in one or more types of cancerous tissue relative to a noncancerous tissue or one or more different types of cancerous tissue, that are methylated in respective nucleic acid sequences). Another example of a bin-level feature is a proportion of sequence reads assigned to a respective bin that have a particular methylation pattern, e.g., that have at least a threshold amount of methylation (e.g., where at least 10%, 15%, 20%, 25%, 30%, 35%, 40%, 45%, 50%, 55%, 60%, 65%, 70%, 75%, 80%, 85%, 90%, 95%, etc., of the putative methylation sites are methylated). Another example of a bin-level feature is a distribution of corresponding probabilities that respective nucleic acid sequences assigned to a respective bin are derived from a cancerous tissue. Another example of a bin-level feature is a summary statistic for the distribution of corresponding probabilities that respective nucleic acid sequences assigned to a respective bin are derived from a cancerous tissue, e.g., a measure of central tendency or a measure of dispersion of the distribution.

In some embodiments, a bin-level methylation ratio refers to a metric (e.g., a proportion, ratio, distribution, measure of central tendency, etc.) for all putative CpG sites that are methylated in the nucleic acid sequences assigned to a respective bin. In some embodiments, a bin-level methylation ratio refers to a metric (e.g., a proportion, ratio, distribution, measure of central tendency, etc.) for a subset of all putative CpG sites that are methylated in the nucleic acid sequences assigned to a respective bin, e.g., a subset of CpG sites that are differentially methylated in one or more types of cancerous tissue relative to a non-cancerous tissue and/or relative to one or more different types of cancerous tissue.

In some embodiments, a bin-level methylation ratio refers to a metric (e.g., a proportion, ratio, distribution, measure of central tendency, etc.) for all cytosine nucleotides that are methylated in the nucleic acid sequences assigned to a respective bin. In some embodiments, a bin-level methylation ratio refers to a metric (e.g., a proportion, ratio, distribution, measure of central tendency, etc.) for a subset of cytosine nucleotides that are methylated in the nucleic acid sequences assigned to a respective bin, e.g., a subset of cytosine nucleotides that are differentially methylated in one or more types of cancerous tissue relative to a non-cancerous tissue and/or relative to one or more different types of cancerous tissue.

In some embodiments, a bin-level methylation ratio refers to a metric (e.g., a proportion, ratio, distribution, measure of central tendency, etc.) of the methylation status for all CHG trinucleotides, where the C is a putative methylation site and H is an A, T, or C nucleotide in the nucleic acid sequences assigned to a respective bin. In some embodiments, a bin-level methylation ratio refers to a metric (e.g., a proportion, ratio, distribution, measure of central tendency, etc.) of the methylation status for a subset of all CHG trinucleotides, where the C is a putative methylation site and H is an A, T, or C nucleotide, in the nucleic acid sequences assigned to a respective bin, e.g., a subset of CHG trinucleotides, where the C is a putative methylation site and H is an A, T, or C nucleotide, that are differentially methylated in one or more types of cancerous tissue relative to a non-cancerous tissue and/or relative to one or more different types of cancerous tissue.

In some embodiments, a bin-level methylation ratio refers to a metric (e.g., a proportion, ratio, distribution, measure of central tendency, etc.) of the methylation status for all CHH trinucleotides, where the C is a putative methylation site and H is an A, T, or C nucleotide, in the nucleic acid sequences assigned to a respective bin. In some embodiments, a bin-level methylation ratio refers to a metric (e.g., a proportion, ratio, distribution, measure of central tendency, etc.) of the methylation status for a subset of all CHH trinucleotides, where the C is a putative methylation site and H is an A, T, or C nucleotide, in the nucleic acid sequences assigned to a respective bin, e.g., a subset of CHH trinucleotides, where the C is a putative methylation site and H is an A, T, or C nucleotide, that are differentially methylated in one or more types of cancerous tissue relative to a non-cancerous tissue and/or relative to one or more different types of cancerous tissue.

In some embodiments, two or more methylation patterns relating to specific cancer states, e.g., particular types of cancer, particular stages of cancer, particular subclonal populations of a cancer, and the like, are deconvolved from methylation sequencing data. In some embodiments, methylation sequencing data is compared to reference methylation patterns, e.g., from training data, and individual signatures are deconvolved from the sequencing data, e.g., by modeling the data in a mixture model, maximum likelihood algorithm, or other trained model of the reference methylation patterns. In some embodiments, individual methylation patterns are learned from specific cell types, e.g., cells having a particular cancer state (e.g., type, stage, mutational profile, etc.). In some embodiments, individual methylation patterns are learned from tissue samples, e.g., from a solid tumor having a particular cancer status (e.g., type, stage, mutational profile, etc.). In some embodiments, patterns from specific cell types work better as training data, e.g., because cancerous tissues display various degrees of heterogeneity.

In some embodiments, methylation patterns and/or classification algorithms are learned from sequencing data from cultured cell lines. In other embodiments, sequencing data from cultured cell lines is excluded because the methylation patterns of cultured cells may not faithfully reflect the methylation profiles of corresponding cells in vivo.

In some embodiments, the methylation pattern used for training a classifier and/or deconvolution algorithm is derived from process-matched sequencing data. For instance, in some embodiments, nucleic acids are prepared from the training tissue sample in the same fashion as nucleic acids from a test sample will be prepared. Similarly, in some embodiments, the sequencing of training samples is performed using the same technique as is used to generate the sequencing data for the test sample. For instance, in some embodiments, both the training data and the test data are prepared using a whole genome enzymatic methylation sequencing methodology, e.g., as compared to a chemical methylation sequencing methodology, such as bisulfite sequencing.

In some embodiments, a bin-level feature relating to the fragmentation pattern of the cell-free DNA is used to train a classification algorithm. For instance, in some embodiments, unique sequence reads are binned based on their position within a reference genome for the subject, as described herein. A metric relating to the distribution of fragment lengths of the sequence reads is then calculated for the bin. For instance, in some embodiments, the length of each fragment sequence is compared to a predetermined threshold length, and the fragment is classified as either a long fragment or a short fragment. A comparison of the number of short fragments to the number of long fragments within the bin (e.g., a ratio of short to long fragments or vice versa) is made to prepare a fragment length metric, e.g., a fragment length ratio, for the bin. Such a fragment-length metric can be used alone or in combination with other metrics, e.g., a methylation feature, a genomic location, etc., to train a classification algorithm. In some embodiments, a fragment-length metric used for training a classification algorithm is processed-matched with a test sample, as differences in fragment length distributions can be attributable to the methodology used to prepare and/or sequence the sample. In some embodiments, a probabilistic, deep learning, and/or admixture model is prepared based on the fragment length distribution metric alone or in combination with any other feature described herein.

In some embodiments, a bin-level feature relating to the coverage ratio of sequences falling within the bin is used to train a model described herein (e.g., a circulating tumor fraction estimation model, a cancer classification model, or, when using an ensemble model, a component model thereof of). For instance, in some embodiments, sequence reads (e.g., raw sequence reads or nucleic acid sequences representing unique DNA fragments after de-duplication) are binned based on their position within a reference genome for the subject, as described herein. A metric relating to the coverage of all of the sequence reads across the bin is then calculated. For example, in some embodiments, a comparison (e.g., a ratio, such as a log 2 ratio) is made between (i) the sequence coverage within the bin by the sequence reads from the test sample, and (ii) the sequence coverage within the bin by sequence reads from one or more (e.g., processed-matched) reference samples. In some embodiments, the algorithm is based on a log ratio, e.g., a log 2 ratio, of average coverage across the bin, e.g., relative to one or more process-matched samples. Such a coverage-based feature can be used alone or in combination with other features, e.g., a methylation feature, a genomic location, fragment-length feature, etc., to train a model described herein. In some embodiments, a probabilistic, deep learning, and/or admixture model is prepared based on such a coverage-based feature.

In yet other embodiments, a methylation feature as described herein is combined with a genomic feature, in order to train a classification algorithm. In some embodiments, a methylation ratio in one or more predetermined promoter regions, one or more enhancer regions, and/or one or more other biologically defined regions is used as a feature for training a classifier, according to the present disclosure. In some embodiments, other epigenomic features are used alone, or in combination with one or more methylation features, in order to train a classification algorithm.

In some embodiments, feature selection is used to identify informative subsets of feature types. For example, in some embodiments, a subset of bins in a plurality of bins, e.g., spanning all, or a majority, of a reference genome for a species of a subject, are identified as particularly informative. For instance, in some embodiments, bins having a defined size, e.g., as described herein, are established and various feature selection methods are used to identify individual bins that are informative of a particular cancer characteristic, e.g., a cancer type, stage, mutational profile, metastatic status, etc. These identified subsets of features are then used to train a classification algorithm, e.g., a probabilistic, deep learning, and/or admixture model. In some embodiments, the feature selection is further biased, and/or limited to, particular biological contexts, e.g., biased or limited to one or more of promoter sequences, enhancer sequences, exons, introns, silencers, intragenic regions with particular properties, etc. Similarly, in some embodiments, particular regions can be excluded from the feature selection process, e.g., telomeric regions, centromeric regions, or other regions believed to not be biologically relevant for the cancer characteristic of interest.

Various methods for feature selection are known in the art. For instance, in some embodiments, features are selected by identifying a statistical difference between a clinical sample, e.g., from a subject having a particular cancer status, and normal sample, e.g., from a subject that does not have that particular cancer status. One example of such a statistical method is the use of Z-scores to identify statistical differences. For example, in one embodiment, a Z-score is determined between the methylation ratio, e.g., as described above, for each bin in a plurality of bins across the genome of the species of the subject. In some embodiments, the selection process is then based upon, at least in part, the difference between samples in the same cohort (e.g., samples with the smallest difference within a cohort are more likely informative than those with larger differences) and/or the difference between samples in different cohorts (e.g., samples with the largest difference between cohorts are more likely informative than those with smaller differences). In some embodiments, PCA analysis is used to identify useful features. For example, in some embodiments PCA analysis is performed on binned methylation ratios, as described herein, and one or more principal components that explain a large amount of the variance across the data set are identified as useful features for a classification algorithm.

In some embodiments, down-sampling is used to identify features with the largest effect on the signature of the data. For instance, starting with a maximal sequence coverage across a region of the reference genome of the subject (e.g., 5×, 10×, 20×, etc.), the coverage can be downsampled by removing sequence reads of the BAMs from the data set in silico. The effect of the downsampling can then be determined for different features and/or across the entire signature. For instance, the effect of data downsampling on signal deconvolution can be used to identify features that are particularly important for deconvolution.

Similarly, in some embodiments, manipulating the tumor fraction of a data set in silico can be used to model a particular cancer signature across different stages of the disease. In this fashion, features that are particularly informative at different tumor fractions can be identified. For instance, if features that are highly informative at high tumor fractions are not informative at lower tumor fractions, a classifier trained only on data from training samples with high tumor fractions may perform poorly on test samples from patients with lower tumor fractions. Accordingly, by considering the cancer state signature across a range of tumor fractions, a classifier that is more robust at all tumor fractions can be trained.

In some embodiments of the component and ensemble models described herein, features (e.g., differentially methylated regions and/or differentially methylated CpG dinucleotides) are identified using a Hidden Markov Model (HMM). For example, stretches of genomic regions that have similar methylation patterns (e.g., methylation levels) can be determined using an HMM. Briefly, HMM models with two or more hidden states can be trained. The emission probability of an HMM describes the probability of an observed methylation pattern (e.g., a methylation level of a genomic region or single CpG site) given a state. Generally, the emission probability of the HMM can be a Gaussian, Beta, or any other probability density function describing localized probability. Likewise, the transition matrix for the HMM can be any matrix having the properties of a stochastic matrix. A transition matrix can be either trained or be posited based on prior expectations. For instance, in some embodiments, a posited transition matrix can be optimized or characterized by a hyperparameter sweep.

In some embodiments, one or more states with low methylation levels may be heuristically combined to represent a “hypomethylated state.” Similarly one or more states with high methylation levels may be heuristically combined to represent a “hypermethylated state.”

Generally, regions where normal or clinical samples share the same HMM hidden state can be identified heuristically. The heuristics may require a certain percentage of samples of the same sample type to have identical HMM state. Further, the heuristics may include rules for i) the range of distance between neighboring CpGs, ii) maximum or minimum number of CpGs within a region, and/or iii) maximum or minimum size of the region in base pairs.

An example of the use of a Hidden Markov Model for the identification of differentially methylated sites in methylation sequencing data is described in Shokoohi F, et al., “A hidden markov model for identifying differentially methylated sites in bisulfite sequencing data,” Biometrics, 75(1):210-21 (2019), which is incorporated by reference herein in its entirety for all purposes.

In some embodiments of the component and ensemble models described herein, features (e.g., differentially methylated regions and/or differentially methylated CpG dinucleotides) are identified using epigenome-wide association analysis (EWAS). In some embodiments of EWAS-mediated feature selection, every feature is tested using logistic regression for association with tumor fraction estimates (e.g., determined using ichorCNA) or known tumor fraction labels. In some embodiments, the dependent variables in the analysis include observed counts of methylated and/or non-methylated cytosines, and the independent variables include tumor fractions (e.g., ichorCNA tumor fraction estimates, in silico simulated tumor fractions, etc.). In some embodiments, where the model accounts for methylation degradation and/or incomplete nucleotide conversion during methylation sequencing, the analysis accounts for one or both of an estimate of the degree of DNA methylation degradation and an estimate of the degree of incomplete nucleotide conversion (e.g., as represented by parameters u and vi, described below). In some embodiments, the tumor fractions (e.g., ichorCNA tumor fraction estimates, in silico simulated tumor fractions, etc.) are logit-transformed to retain the linear relationship between dependent and independent variables.

From the results of the EWAS, a set of methylation features (e.g., differentially methylated CpG dinucleotides and/or differentially methylated genomic regions) are selected for use as markers to estimate tumor fraction. In some embodiments, the selected features should be significantly associated with the tumor fraction labels, e.g., ichorCNA tumor fraction estimates. In some embodiments, the selected features should explain observed variability in methylation levels well, e.g., as assessed by high values for McFadden's R². In some embodiments, the selected features should be roughly balanced between CpG sites that are hypo/hyper-methylated in cancerous tissues.

In some embodiments, methylation data used in the component models described herein, e.g., for estimating circulating tumor fraction, is corrected to account for DNA methylation (DNAm) degradation and/or incomplete nucleotide conversion prior to methylation sequencing. In some embodiments, the correction is performed against a set of control features that include CpG dinucleotides and/or genomic regions that are invariantly methylated in cancerous and non-cancerous tissues. Generally, any of the feature selection methodologies described herein can be used to select biologically invariant features. However, rather than selecting for features (e.g., CpG dinucleotides and/or genomic regions) that are differentially methylated in cancerous tissues and non-cancerous tissues, features that are similarly methylated in cancerous and non-cancerous tissues are selected. That is, the features have the same methylation level across all tissues present in the cancerous and non-cancerous training samples. These features will help estimate the degree of degradation and batch effects independently of tumor fraction because all observed variability at these features would either come from DNAm degradation (presumably through the loss of methyl-groups) or incomplete enzymatic methyl-conversion. In some embodiments, the control features are roughly balanced between invariant hypo- and hyper-methylated CpG dinucleotides and/or genomic regions.

In some embodiments, feature set used for a component model described herein contains at least 50, at least 100, at least 150, at least 200, at least 250, at least 300, at least 400, at least 500, at least 600, at least 700, at least 800, at least 900, at least 1000, at least 2500, at least 5000, at least 10,000, at least 15,000, at least 20,000, at least 25,000, at least 30,000, at least 40,000, at least 50,000, or more features. In some embodiments, the selected feature set should contain no more than 1,000,000, 750,000, 500,000, 250,000, 100,000, 75,000, 50,000, 40,000, 30,000, 25,000, 20,000, 15,000, 10,000, 7500, 5000, 2500, 2000, 1500, 1000, 900, 800, 750, or fewer features.

The skilled artisan will appreciate that, in some instances, the use of different training data sets may yield different results. These differences may arise, for example, when different criteria are used to select the training population, e.g., different inclusion and/or exclusion criteria such as cancer type, personal characteristics (e.g., age, gender, ethnicity, family history, smoking status, etc.), or simply by using a smaller or larger data set.

The skilled artisan will also appreciate that some features will be more informative than other features in a particular classifier. One measure of the predictive power of respective features in a classifier based on multiple features is the regression coefficient calculated for the features during training of the model. Regression coefficients describe the relationship between each feature and the response of the model. The coefficient value represents the mean change in the response given a one-unit increase in the feature value. As such, at least for variables of the same type, the magnitude, e.g., absolute value, of a regression coefficient is correlated with the importance of the feature in the model. That is, the higher the magnitude of the regression coefficient, the more important the variable is to the model. As such, in some embodiments, a feature set is selected based, at least in part, upon the importance of the respective features in one or more classification models. For instance, in some embodiments, one or more genes with lower predictive power in a classification model may be left out during classifier training.

In some embodiments, the size of the feature set may be affected by which features are included and/or excluded. For instance, in some embodiments, if particular features having high predictive power are included in a classification model, fewer total features may be included in the model. Similarly, in some embodiments, if features having high predictive power are excluded from the classification model, more of the other features may be included in the model. In some embodiments, other metrics are also available for evaluating the importance of a feature in a model, such as standardized regression coefficients and change in R-squared when the feature is added to the model last.

When selecting a feature set, the skilled artisan will also consider the degree to which features are correlated to each other. Correlation is a statistical measure of how linearly dependent two variables are upon each other. As such, two correlated features provide duplicative information to a predictive model, which can be detrimental to a classifier. As such, there are several reasons why a correlated feature may be excluded from a model. For instance, removing a correlated feature will make the algorithm faster, as the larger the number of features in a classifier the more computations that need to be made. Removing a correlated feature may also remove harmful bias, arising from the correlation, from a model. Finally, removing a correlated feature may make the model more interpretable. In some embodiments, the selection to remove one or the other feature of a correlated feature set is informed by predictive powers of the two features, e.g., their respective regression coefficients.

Some MLA may identify features of importance and identify a coefficient, or weight, to them. The coefficient may be multiplied with the occurrence frequency of the feature to generate a score, and once the scores of one or more features exceed a threshold, certain classifications may be predicted by the MLA. A coefficient schema may be combined with a rule-based schema to generate more complicated predictions, such as predictions based upon multiple features. For example, ten key features may be identified across different classifications. A list of coefficients may exist for the key features, and a rule set may exist for the classification. A rule set may be based upon the number of occurrences of the feature, the scaled weights of the features, or other qualitative and quantitative assessments of features encoded in logic known to those of ordinary skill in the art.

In other MLA, features may be organized in a binary tree structure. For example, key features which distinguish between the most classifications may exist as the root of the binary tree and each subsequent branch in the tree until a classification may be awarded based upon reaching a terminal node of the tree. For example, a binary tree may have a root node which tests for a first feature. The occurrence or non-occurrence of this feature must exist (the binary decision), and the logic may traverse the branch which is true for the item being classified.

Additional rules may be based upon thresholds, ranges, or other qualitative and quantitative tests. While supervised methods are useful when the training dataset has many known values or annotations, some datasets (e.g., EMR/EHR documents) may not include annotations. When exploring large amounts of unlabeled data, unsupervised methods are useful for binning/bucketing instances in the data set. A single instance of the above models, or two or more such instances in combination, may constitute a model for the purposes of models, artificial intelligence, neural networks, or machine learning algorithms, herein.

In some embodiments, a classifier used in the methods described herein is a logistic regression algorithm, a neural network algorithm, a convolutional neural network algorithm, a support vector machine (SVM) algorithm, a Naive Bayes algorithm, a nearest neighbor algorithm, a boosted trees algorithm, a random forest algorithm, a decision tree algorithm, a clustering algorithm, or a combination thereof.

In some embodiments, one or more of the component models described herein is trained using a Markov Chain Monte Carlo (MCMC) methodology.

In one embodiment, one or more of the component models described herein is a probabilistic model using methylation features that accounts for DNA methylation degradation and/or incomplete nucleotide conversion prior to methylation sequencing. In one embodiment, the probabilistic model models counts of methylated Mᵢⱼ and unmethylated Uᵢⱼ cytosines for feature i in sample j in a mixture of two binomial distributions using a mixture proportion (a weight) wᵢ, according to the relationship:

$$P ( {U}_{ij} , {M}_{ij} ) = {w}_{i} \cdot Binomial ( {U}_{ij} , {M}_{ij} \vert {p}_{i j} ) + {( 1 - w )}_{i} \cdot Binomial ( {U}_{ij} , {M}_{ij} \vert {q}_{i j} ) .$$

Briefly, the assumptions of the model are that:
μⱼ represents the rate of originally methylated cytosines that are observed as unmethylated, either because of DNAm degradation or incomplete protection by TET2. This rate varies from sample to sample.
vⱼ represents the rate of originally unmethylated cytosines that are observed as methylated as the result of incomplete nucleotide conversion (e.g., by APOBEC). This rate varies from sample to sample.
parameters μⱼ and vⱼ, the rate of unwanted methylated-to-unmethylated transition and unmethylated-to-methylated transition for sample j, respectively, can be estimated based on features (e.g., CpG dinucleotides and/or genomic regions) that were identified to be similarly methylated in cancerous and non-cancerous tissues, e.g., as described above in the section titled “Multi-feature Classifiers and Machine Learning.”
Counts of (un)methylated cytosines, Uᵢⱼ and Mᵢⱼ for feature i and sample j, are generated by a Binomial distribution.
The success probability pᵢⱼ of the Binomial distribution (counting methylated cytosines as successes) depends on several factors.
   For “invariant” features, the success probability depends on the feature-specific methylation level invariantᵢ and the degree of DNAm degradation and methyl-conversion

$${p}_{i j} = {invariant}_{i} \cdot ( 1 - {μ}_{j} ) + ( 1 - {invariant}_{i} ) \cdot {v}_{j}$$

$$P ( {U}_{ij} , {M}_{ij} ) = Binomial ( {U}_{ij} , {M}_{ij} \vert {p}_{i j} )$$

For the other features, the success probability is a mixture of DNA from tumor cells with a methylation level tumor and from normal cells with methylation level normal; and mixture proportions tfᵢ.

$${{p}^{′}}_{i j} = {tumor}_{i} \cdot {tf}_{j} + {normal}_{i} \cdot ( 1 - {tf}_{j} )$$

$${p}_{i j} = {{p}^{′}}_{i j} \cdot ( 1 - {μ}_{j} ) + ( 1 - {{p}^{′}}_{i j} ) \cdot {v}_{j}$$

But a feature may not be informative in all samples. If it is not, its success probability is qᵢⱼ=normalᵢ·(1−μⱼ)+ (1−normalᵢ)·vⱼ

The data-generating process outlined above is described using the Stan framework, a probabilistic programming language and a program that generates a Hamiltonian Monte Carlo sampler in C++ from a model described in Stan. Stan can be used via R or Python bindings. For example, in some embodiments, the model is trained using Stan by:
1. Running a Hamiltonian Monte Carlo sampler on a training dataset, e.g., containing methylation feature data from liquid biopsy samples from training subjects with cancer and training subjects without cancer for (i) a set of differentially methylated features (e.g., differentially methylated CpG dinucleotides and/or differentially methylated genomic regions, for example as identified using one or more feature selection methodologies described above in the section titled “Multi-feature Classifiers and Machine Learning”), and (ii) a set of invariantly methylated features (e.g., CpG dinucleotides and/or genomic regions that are similarly methylated in cancerous and non-cancerous tissues, for example as identified using one or more feature selection methodologies described above in the section titled “Multi-feature Classifiers and Machine Learning”).
2. Starting several Markov chains and checking for convergence.
3. Extracting the mean posterior estimates for the following parameters: normalᵢ, tumorᵢ, wᵢ, invariantᵢ

Accordingly, in some embodiments, circulating tumor fraction estimates can be generated for test samples by: (i) using a set of invariantly methylated features (e.g., CpG dinucleotides and/or genomic regions that are similarly methylated in cancerous and non-cancerous tissues, for example as identified using one or more feature selection methodologies described above in the section titled “Multi-feature Classifiers and Machine Learning”) to estimate sample-specific parameters, μⱼ and vⱼ, and (ii) estimating the tumor fraction of the sample (tfᵢ) by choosing the value that maximizes the likelihood function:

$$\begin{matrix}L = {\prod }_{i} & Binomial ( {U}_{ij} , {M}_{ij} \vert {p}_{i j} )\end{matrix} ,$$

$$where :$$

$${{p}^{′}}_{i j} = {tumor}_{i} \cdot {tf}_{j} + {normal}_{i} \cdot ( 1 - {tf}_{j} ) , and$$

$${p}_{i j} = {{p}^{′}}_{i j} \cdot ( 1 - {μ}_{j} ) + ( 1 - {{p}^{′}}_{i j} ) \cdot {v}_{j} .$$

In some embodiments, a probabilistic model is used in the methods and systems described herein, e.g., as a component model of an ensemble classifier or circulating tumor fraction estimation model. Probabilistic models employ random variables and probability distributions to a model for a phenomenon, e.g., the presence of a cancer state, circulating tumor fraction, etc. Probabilistic models provide a probability distribution as a solution. Generally, probabilistic models can be classified as either graphical models (such as Bayesian networks, causal inference models, and Markov networks) or Stochastic models.

Probabilistic graphical models (PGMs) are probabilistic models for which a graph expresses a conditional dependence structure between random variables, encoding a distribution over a multi-dimensional space. One type of PGM is a Bayesian network, which is probabilistic graphical model that represents a set of variables and their conditional dependencies via a directed acyclic graph (DAG), according to Bayesian analysis. Briefly, given data x and parameter θ, Bayesian analysis uses a prior probability (a prior) p(θ) and a likelihood p(x|θ) to compute a posterior probability p(θ|x)∝p(x|θ)p(θ). Methods for learning Bayesian Networks are described, for example, in Castillo E, et al., “Learning Bayesian Networks,” Expert Systems and Probabilistic Network Models, Monographs in computer science, New York: Springer-Verlag, pp. 481-528, ISBN 978-0-387-94858-4, which is incorporated herein by reference, in its entirety, for all purposes. Another type of PGM is a Markov network, which is a set of random variables having a Markov property described by an undirected graph. Markov properties include pairwise Markov properties, in which any two non-adjacent variables are conditionally independent given all other variables, local Markov properties, in which a variable is conditionally independent of all other variables given its neighbors, and global Markov properties, in which any two subsets of variables are conditionally independent given a separating subset.

Stochastic probabilistic models model pseudo-randomly changing systems, assuming that future states depend only on a current state, not the events that occurred before the current state, otherwise known as the Markov property. Stochastic probabilistic models include Markov chains and Hidden Markov models (HMM). Markov chains are models describing a sequence of possible events in which the probability of each event depends only on the state attained in the previous event. For information on learning and application of Markov chains see, for example, Gagniuc, Paul A. (2017). Markov Chains: From Theory to Implementation and Experimentation. USA, NJ: John Wiley & Sons. pp. 1-235. ISBN 978-1-119-38755-8, which is incorporated herein by reference, in its entirety, for all purposes. Hidden Markov models (HMM) assume that a property X is dependent upon an unobservable (“hidden”) state Y that can be learned based on observation of the property. For review of Hidden Markov models see, for example, Rabiner and Juang, “An introduction to hidden Markov models,” IEEE ASSP Magazine, 3(1):4-16 (1986), which is incorporated herein by reference, in its entirety, for all purposes.

In some embodiments, a deep learning model, is used in the methods and systems described herein, e.g., as a component model of an ensemble classifier or circulating tumor fraction estimation model. Deep learning models use multiple layers to extract higher-level features from input data.

Neural networks. In some embodiments, the deep learning model is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in analyzing the methylation, copy number state, and/or fragment length metrics from a liquid biopsy sample to inform identification of a circulating tumor fraction and/or a cancer status for the subject. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing methylation, copy number state, and/or fragment length metrics from a liquid biopsy sample to inform identification of a circulating tumor fraction and/or a cancer status for the subject.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,”’ CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

In some embodiments, a mixture model, also referred to herein as an admixture model, is used in the methods and systems described herein, e.g., as a component model of an ensemble classifier or circulating tumor fraction estimation model. Mixture models are probabilistic model for representing the presence of subpopulations within an overall population, without requiring that an observed data set should identify the sub-population to which an individual observation belongs. Given a sampling of parameter data from a mixture of distributions, e.g., fragment lengths, copy number states, and/or methylation states for cfDNA fragments derived from either cancerous cells of non-cancerous cells, and model distributions of the parameters over each distribution separately, several techniques can be used to determine the parameters of the particular mixture of distributions. These techniques include maximum likelihood estimation (e.g., vial expectation maximization), application of Bayes' theorem on posterior sampling of the mixture of distributions (e.g., via a Markov chain Monte Carlo algorithm such as Gibbs sampling), moment matching, and several graphical methodologies. For a review of the use of mixture models see, for example, Titterington, D et al., “Statistical Analysis of Finite Mixture Distributions,” Wiley ISBN 978-0-471-90763-3 (1985), which is incorporated herein by reference, in its entirety, for all purposes.

Logistic regression algorithms suitable for use as classifiers are disclosed, for example, in Agresti, An Introduction to Categorical Data Analysis, 1996, Chapter 5, pp. 103-144, John Wiley & Son, New York, which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. A neural network has a layered structure that includes a layer of input units (and the bias) connected by a layer of weights to a layer of output units. For regression, the layer of output units typically includes just one output unit. However, neural networks can handle multiple quantitative responses in a seamless fashion. In multilayer neural networks, there are input units (input layer), hidden units (hidden layer), and output units (output layer). There is, furthermore, a single bias unit that is connected to each unit other than the input units. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training set (e.g., a first and second cancer condition of each respective subject in a plurality of subjects) with a hyperplane that is maximally distant from the labeled data. For cases in which no linear separation is possible, SVMs can work in combination with the technique of ‘kernels, which automatically realize a non-linear mapping to a feature space. The hyperplane found by the SVM in feature space corresponds to a non-linear decision boundary in the input space.

Naïve Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. Decision trees algorithms suitable for use as classifiers are described in, for example, Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used as a classifier is a classification and regression tree (CART). Other examples of specific decision tree algorithms that can be used as classifiers include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As set forth in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined. Similarity measures are discussed in Section 6.7 of Duda 1973, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda 1973, clustering does not require the use of a distance metric. For example, a nonmetric similarity function s (x, x′) can be used to compare two vectors x and x′. Conventionally, s (x, x′) is a symmetric function whose value is large when x and x′ are somehow “similar.” An example of a nonmetric similarity function s (x, x′) is provided on page 216 of Duda 1973.

Once a method for measuring “similarity” or “dissimilarity” between points in a dataset has been selected, clustering makes use of a criterion function that measures the clustering quality of any partition of the data. Partitions of the dataset that extremize the criterion function are used to cluster the data. See page 217 of Duda 1973. Criterion functions are discussed in Section 6.8 of Duda 1973. More recently, Duda et al., Pattern Classification, 2^{nd} edition, John Wiley & Sons, Inc. New York, has been published. Pages 537-563 describe clustering in detail. More information on clustering techniques suitable for use as classifiers are disclosed in Kaufman and Roussecuw, 1990, Finding Groups in Data: An Introduction to Cluster Analysis, Wiley, New York, N. Y.; Everitt, 1993, Cluster analysis (3 d ed.), Wiley, New York, N.Y.; and Backer, 1995, Computer-Assisted Reasoning in Cluster Analysis, Prentice Hall, Upper Saddle River, N.J. Particular exemplary clustering techniques that can be used as classifiers include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

In some embodiments, a classifier is a nearest neighbor algorithm. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

Further details on using analysis results from sequencing data to classify a cancer condition of a biological sample and/or a subject are disclosed in U.S. Patent Application No. 62/810,849, titled “SYSTEMS AND METHODS FOR USING SEQUENCING DATA FOR PATHOGEN DETECTION,” filed Feb. 26, 2019, and U.S. patent application Ser. No. 16/789,363 (PCT/US20/180002), titled “AN INTEGRATED MACHINE-LEARNING FRAMEWORK TO ESTIMATE HOMOLOGOUS RECOMBINATION DEFICIENCY,” filed Feb. 12, 2020, each of which is hereby incorporated herein by reference in its entirety.

### Cohorts and Clinical Trials:

In some embodiments, a plurality of biological subjects is a clinical cohort, e.g., a group of participants in a clinical trial or study. In some embodiments, the plurality of subjects in the cohort comprises at least 10, at least 20, at least 30, at least 40, at least 50, at least 100, at least 200, at least 500, at least 1000, at least 2000, or at least 5000 subjects.

In some embodiments, each of the subjects in the plurality of biological subjects (e.g., cohort) has a same type of cancer (e.g., a type of cancer originating from a same site within the body, e.g., NSCLC). In some embodiments, each of the subjects in the plurality of biological subjects has a cancer that is associated with a set of cancers.

In some embodiments, the plurality of biological subjects comprises one or more subsets of subjects with a respective cancer condition, where the respective cancer condition is not represented in any other subset of subjects in the plurality of subjects. For example, in some embodiments, a first subject (and/or a subset of subjects) in a plurality of subjects has a first cancer condition, and a second subject (and/or a subset of subjects) in a plurality of subjects has a second cancer condition.

In some embodiments, the first subset of subjects and the second subset of subjects each comprise the same number of subjects. For example, in some such embodiments, the plurality of subjects comprises one hundred subjects, the first subset of subjects (e.g., with the first cancer condition) comprises twenty subjects, and the second subset of subjects (e.g., with the second cancer condition) comprises twenty subjects. In other embodiments, the plurality of subjects comprises one thousand subjects, the first subset of subjects comprises one hundred subjects, and the second subset of subjects comprises one hundred subjects.

In some alternative embodiments, the first subset of subjects and the second subset of subjects comprise different numbers of subjects (e.g., a greater number of subjects in the plurality of subjects have the first cancer condition than the second cancer condition). For instance, in some embodiments, more than ten percent, more than twenty percent, more than thirty percent, more than forty percent, more than fifty percent, more than sixty percent, more than seventy percent, more than eighty percent, or more than ninety percent of the subjects in the plurality of subjects have the first cancer condition while the remainder have the second cancer condition.

### Training and Test Subjects:

In some embodiments, a plurality of subjects (e.g., a cohort) is of sufficient size to develop a classifier that has suitable performance for screening subjects to ascertain whether they have a first or second cancer condition (see Cohorts, above).

In some embodiments, a plurality of subjects comprises training subjects (e.g., subjects for which the cancer condition is known). In some embodiments, training subjects can be used to train a classifier to detect or distinguish a cancer condition.

In some embodiments, a plurality of subjects comprises test subjects (e.g., subjects for which the cancer condition is unknown). For example, in typical instances, a test subject is a subject for which it has not been confirmed whether the subject has a first or second cancer condition.

In some such embodiments, a trained classifier is used to classify a test subject (e.g., by detecting or distinguishing the cancer condition of the test subject). In some such embodiments, a test subject is a subject that was not used to train the classifier.

### Example Methods

Now that details of a system 100 for providing clinical support for personalized cancer therapy, e.g., with improved methods for estimating circulating tumor fraction and cancer monitoring using low-pass whole genome methylation sequencing, have been disclosed, details regarding processes and features of the system, in accordance with various embodiments of the present disclosure, are disclosed below. Specifically, example processes are described below with reference to FIGS. 2A, 3, 4A-4C, 5, 6, 7, 8, and 9. In some embodiments, such processes and features of the system are carried out by modules 118, 120, 140, 160, and/or 170, as illustrated in FIG. 1A. Referring to these methods, the systems described herein (e.g., system 100) include instructions for determining for estimating circulating tumor fraction and cancer monitoring using low-pass whole genome methylation sequencing that are improved compared to conventional methods for estimating circulating tumor fraction and cancer monitoring.

### FIG. 2B: Distributed Diagnostic and Clinical Environment

In some aspects, the methods described herein for providing clinical support for personalized cancer therapy are performed across a distributed diagnostic/clinical environment, e.g., as illustrated in FIG. 2B. However, in some embodiments, the improved methods described herein for estimating circulating tumor fraction and cancer monitoring using low-pass whole genome methylation sequencing, are performed at a single location, e.g., at a single computing system or environment, although ancillary procedures supporting the methods described herein, and/or procedures that make further use of the results of the methods described herein, may be performed across a distributed diagnostic/clinical environment.

FIG. 2B illustrates an example of a distributed diagnostic/clinical environment 210. In some embodiments, the distributed diagnostic/clinical environment is connected via communication network 105. In some embodiments, one or more biological samples, e.g., one or more liquid biopsy samples, solid tumor biopsy, normal tissue samples, and/or control samples, are collected from a subject in clinical environment 220, e.g., a doctor's office, hospital, or medical clinic, or at a home health care environment (not depicted). Advantageously, while solid tumor samples should be collected within a clinical setting, liquid biopsy samples can be acquired in a less invasive fashion and are more easily collected outside of a traditional clinical setting. In some embodiments, one or more biological samples, or portions thereof, are processed within the clinical environment 220 where collection occurred, using a processing device 224, e.g., a nucleic acid sequencer for obtaining sequencing data, a microscope for obtaining pathology data, a mass spectrometer for obtaining proteomic data, etc. In some embodiments, one or more biological samples, or portions thereof are sent to one or more external environments, e.g., sequencing lab 230, pathology lab 240, and/or molecular biology lab 250, each of which includes a processing device 234, 244, and 254, respectively, to generate biological data 121 for the subject. Each environment includes a communications device 222, 232, 242, and 252, respectively, for communicating biological data 121 about the subject to a processing server 262 and/or database 264, which may be located in yet another environment, e.g., processing/storage center 260. Thus, in some embodiments, different portions of the systems and methods described herein are fulfilled by different processing devices located in different physical environments.

Accordingly, in some embodiments, a method for providing clinical support for personalized cancer therapy, e.g., with improved methods for estimating circulating tumor fraction and cancer monitoring using low-pass whole genome methylation sequencing, is performed across one or more environments, as illustrated in FIG. 2B. For instance, in some such embodiments, a liquid biopsy sample is collected at clinical environment 220 or in a home healthcare environment. The sample, or a portion thereof, is sent to sequencing lab 230 where raw sequence reads 123 of nucleic acids in the sample are generated by sequencer 234. The raw sequencing data 123 is communicated, e.g., from communications device 232, to database 264 at processing/storage center 260, where processing server 262 extracts features from the sequence reads by executing one or more of the processes in bioinformatics module 140, thereby generating genomic features 131 for the sample. Processing server 262 may then analyze the identified features by executing one or more of the processes in feature analysis module 160, thereby generating clinical assessment 139, including a clinical report 139-3. A clinician may access clinical report 139-3, e.g., at processing/storage center 260 or through communications network 105, via recommendation validation module 167. After final approval, clinical report 139-3 is transmitted to a medical professional, e.g., an oncologist, at clinical environment 220, who uses the report to support clinical decision making for personalized treatment of the patient's cancer.

### FIG. 2A: Example Workflow for Precision Oncology

FIG. 2A is a flowchart of an example workflow 200 for collecting and analyzing data in order to generate a clinical report 139 to support clinical decision making in precision oncology. Advantageously, the methods described herein improve this process, for example, by improving various steps implemented during feature extraction 206, including estimating circulating tumor fraction and cancer monitoring using low-pass whole genome methylation sequencing.

Briefly, the workflow begins with patient intake and sample collection 201, where one or more liquid biopsy samples, one or more tumor biopsy, and one or more normal and/or control tissue samples are collected from the patient (e.g., at a clinical environment 220 or home healthcare environment, as illustrated in FIG. 2B). In some embodiments, personal data 126 corresponding to the patient and a record of the one or more biological samples obtained (e.g., patient identifiers, patient clinical data, sample type, sample identifiers, cancer conditions, etc.) are entered into a data analysis platform, e.g., test patient data store 120. Accordingly, in some embodiments, the methods disclosed herein include obtaining one or more biological samples from one or more subjects, e.g., cancer patients. In some embodiments, the subject is a human, e.g., a human cancer patient.

In some embodiments, one or more of the biological samples obtained from the patient are a biological liquid sample, also referred to as a liquid biopsy sample. In some embodiments, one or more of the biological samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, blood samples are collected from patients in commercial blood collection containers, e.g., using a PAXgene® Blood DNA Tubes. In some embodiments, saliva samples are collected from patients in commercial saliva collection containers, e.g., using an Oragene® DNA Saliva Kit.

In some embodiments, the liquid biopsy sample has a volume of from about 1 mL to about 50 mL. For example, in some embodiments, the liquid biopsy sample has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, or greater.

Liquid biopsy samples include cell free nucleic acids, including cell-free DNA (cfDNA). As described above, cfDNA isolated from cancer patients includes DNA originating from cancerous cells, also referred to as circulating tumor DNA (ctDNA), cfDNA originating from germline (e.g., healthy or non-cancerous) cells, and cfDNA originating from hematopoietic cells (e.g., white blood cells). The relative proportions of cancerous and non-cancerous cfDNA present in a liquid biopsy sample varies depending on the characteristics (e.g., the type, stage, lineage, genomic profile, etc.) of the patient's cancer. As used herein, the ‘tumor burden’ of the subject refers to the percentage cfDNA that originated from cancerous cells.

As described herein, cfDNA is a particularly useful source of biological data for various implementations of the methods and systems described herein, because it is readily obtained from various body fluids. Advantageously, use of bodily fluids facilitates serial monitoring because of the case of collection, as these fluids are collectable by non-invasive or minimally-invasive methodologies. This is in contrast to methods that rely upon solid tissue samples, such as biopsies, which often times require invasive surgical procedures. Further, because bodily fluids, such as blood, circulate throughout the body, the cfDNA population represents a sampling of many different tissue types from many different locations.

In some embodiments, a liquid biopsy sample is separated into two different samples. For example in some embodiments, a blood sample is separated into a blood plasma sample, containing cfDNA, and a buffy coat preparation, containing white blood cells.

In some embodiments, a plurality of liquid biopsy samples is obtained from a respective subject at intervals over a period of time (e.g., using serial testing). For example, in some such embodiments, the time between obtaining liquid biopsy samples from a respective subject is at least 1 day, at least 2 days, at least 1 week, at least 2 weeks, at least 1 month, at least 2 months, at least 3 months, at least 4 months, at least 6 months, or at least 1 year.

In some embodiments, one or more biological samples collected from the patient is a solid tissue sample, e.g., a solid tumor sample or a solid normal tissue sample. Methods for obtaining solid tissue samples, e.g., of cancerous and/or normal tissue are known in the art, and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, e.g., a swab, in the subjects mouth and rubbing it against their check, e.g., for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

The biological samples collected from the patient are, optionally, sent to various analytical environments (e.g., sequencing lab 230, pathology lab 240, and/or molecular biology lab 250) for processing (e.g., data collection) and/or analysis (e.g., feature extraction). Wet lab processing 204 may include a steps of cataloguing samples (e.g., accessioning), examining clinical features of one or more samples (e.g., pathology review), and nucleic acid sequence analysis (e.g., extraction, library prep, capture+hybridize, pooling, and sequencing). In some embodiments, the workflow includes clinical analysis of one or more biological samples collected from the subject, e.g., at a pathology lab 240 and/or a molecular and cellular biology lab 250, to generate clinical features such as pathology features 128-3, imaging data 128-3, and/or tissue culture/organoid data 128-3.

In some embodiments, the pathology data 128-1 collected during clinical evaluation includes visual features identified by a pathologist's inspection of a specimen (e.g., a solid tumor biopsy), e.g., of stained H&E or IHC slides. In some embodiments, the sample is a solid tissue biopsy sample. In some embodiments, the tissue biopsy sample is a formalin-fixed tissue (FFT), e.g., a formalin-fixed paraffin-embedded (FFPE) tissue. In some embodiments, the tissue biopsy sample is an FFPE or FFT block. In some embodiments, the tissue biopsy sample is a fresh-frozen tissue biopsy. The tissue biopsy sample can be prepared in thin sections (e.g., by cutting and/or affixing to a slide), to facilitate pathology review (e.g., by staining with immunohistochemistry stain for IHC review and/or with hematoxylin and cosin stain for H&E pathology review). For instance, analysis of slides for H&E staining or IHC staining may reveal features such as tumor infiltration, programmed death-ligand 1 (PD-L1) status, human leukocyte antigen (HLA) status, or other immunological features.

In some embodiments, a liquid sample (e.g., blood) collected from the patient (e.g., in EDTA-containing collection tubes) is prepared on a slide (e.g., by smearing) for pathology review. In some embodiments, macrodissected FFPE tissue sections, which may be mounted on a histopathology slide, from solid tissue samples (e.g., tumor or normal tissue) are analyzed by pathologists. In some embodiments, tumor samples are evaluated to determine, e.g., the tumor purity of the sample, the percent tumor cellularity as a ratio of tumor to normal nuclei, etc. For each section, background tissue may be excluded or removed such that the section meets a tumor purity threshold, e.g., where at least 20% of the nuclei in the section are tumor nuclei, or where at least 25%, 30%, 40%, 50%, 60%, 70%, 80%, 90%, or more of the nuclei in the section are tumor nuclei.

In some embodiments, pathology data 128-1 is extracted, in addition to or instead of visual inspection, using computational approaches to digital pathology, e.g., providing morphometric features extracted from digital images of stained tissue samples. A review of digital pathology methods is provided in Bera, K. et al., Nat. Rev. Clin. Oncol., 16:703-15 (2019), the content of which is hereby incorporated by reference, in its entirety, for all purposes. In some embodiments, pathology data 128-1 includes features determined using machine learning algorithms to evaluate pathology data collected as described above.

Further details on methods, systems, and algorithms for using pathology data to classify cancer and identify targeted therapies are discussed, for example, in are discussed, for example, in U.S. patent application Ser. No. 16/830,186, filed on Mar. 25, 2020, and U.S. Provisional Application No. 63/007,874, filed on Apr. 9, 2020, the contents of which are hereby incorporated by reference, in their entireties, for all purposes.

In some embodiments, imaging data 128-2 collected during clinical evaluation includes features identified by review of in-vitro and/or in-vivo imaging results (e.g., of a tumor site), for example a size of a tumor, tumor size differentials over time (such as during treatment or during other periods of change). In some embodiments, imaging data 128-2 includes features determined using machine learning algorithms to evaluate imaging data collected as described above.

Further details on methods, systems, and algorithms for using medical imaging to classify cancer and identify targeted therapies are discussed, for example, in are discussed, for example, in U.S. patent application Ser. No. 16/830,186, filed on Mar. 25, 2020, and U.S. Provisional Application No. 63/007,874, filed on Apr. 9, 2020, the contents of which are hereby incorporated by reference, in their entireties, for all purposes.

In some embodiments, tissue culture/organoid data 128-3 collected during clinical evaluation includes features identified by evaluation of cultured tissue from the subject. For instance, in some embodiments, tissue samples obtained from the patients (e.g., tumor tissue, normal tissue, or both) are cultured (e.g., in liquid culture, solid-phase culture, and/or organoid culture) and various features, such as cell morphology, growth characteristics, genomic alterations, and/or drug sensitivity, are evaluated. In some embodiments, tissue culture/organoid data 128-3 includes features determined using machine learning algorithms to evaluate tissue culture/organoid data collected as described above. Examples of tissue organoid (e.g., personal tumor organoid) culturing and feature extractions thereof are described in U.S. Provisional Application Ser. No. 62/924,621, filed on Oct. 22, 2019, and U.S. patent application Ser. No. 16/693,117, filed on Nov. 22, 2019, the contents of which are hereby incorporated by reference, in their entireties, for all purposes.

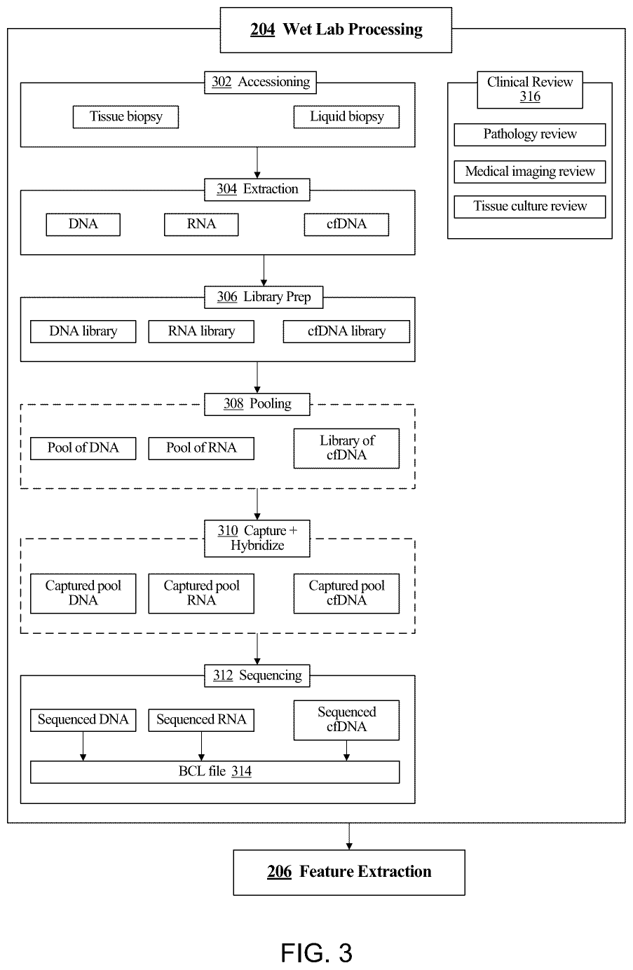

Nucleic acid sequencing of one or more samples collected from the subject is performed, e.g., at sequencing lab 230, during wet lab processing 204. An example workflow for nucleic acid sequencing is illustrated in FIG. 3. In some embodiments, the one or more biological samples obtained at the sequencing lab 230 are accessioned (302), to track the sample and data through the sequencing process.

Next, nucleic acids, e.g., RNA and/or DNA are extracted (304) from the one or more biological samples. Methods for isolating nucleic acids from biological samples are known in the art, and are dependent upon the type of nucleic acid being isolated (e.g., cfDNA, DNA, and/or RNA) and the type of sample from which the nucleic acids are being isolated (e.g., liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced and the sequencing technology being used.

For instance, many techniques for DNA isolation, e.g., genomic DNA isolation, from a tissue sample are known in the art, such as organic extraction, silica adsorption, and anion exchange chromatography. Likewise, many techniques for RNA isolation, e.g., mRNA isolation, from a tissue sample are known in the art. For example, acid guanidinium thiocyanate-phenol-chloroform extraction (see, for example, Chomczynski and Sacchi, 2006, Nat Protoc, 1 (2): 581-85, which is hereby incorporated by reference herein), and silica bead/glass fiber adsorption (see, for example, Pocckh, T. et al., 2008, Anal Biochem., 373 (2): 253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, e.g., fresh, frozen, formalin-fixed, paraffin-embedded (FFPE), and the type of nucleic acid analysis that is to be performed.

In some embodiments where the biological sample is a liquid biopsy sample, e.g., a blood or blood plasma sample, cfDNA is isolated from blood samples using commercially available reagents, including proteinase K, to generate a liquid solution of cfDNA.

In some embodiments, isolated DNA molecules are mechanically sheared to an average length using an ultrasonicator (for example, a Covaris ultrasonicator). In some embodiments, isolated nucleic acid molecules are analyzed to determine their fragment size, e.g., through gel electrophoresis techniques and/or the use of a device such as a LabChip GX Touch. The skilled artisan will know of an appropriate range of fragment sizes, based on the sequencing technique being employed, as different sequencing techniques have differing fragment size requirements for robust sequencing. In some embodiments, quality control testing is performed on the extracted nucleic acids (e.g., DNA and/or RNA), e.g., to assess the nucleic acid concentration and/or fragment size. For example, sizing of DNA fragments provides valuable information used for downstream processing, such as determining whether DNA fragments require additional shearing prior to sequencing.

Wet lab processing 204 then includes preparing a nucleic acid library from the isolated nucleic acids (e.g., cfDNA, DNA, and/or RNA). For example, in some embodiments, DNA libraries (e.g., gDNA and/or cfDNA libraries) are prepared from isolated DNA from the one or more biological samples. In some embodiments, the DNA libraries are prepared using a commercial library preparation kit, e.g., the KAPA Hyper Prep Kit, a New England Biolabs (NEB) kit, or a similar kit.

In some embodiments, during library preparation, adapters (e.g., UDI adapters, such as Roche SeqCap dual end adapters, or UMI adapters such as full length or stubby Y adapters) are ligated onto the nucleic acid molecules. In some embodiments, the adapters include unique molecular identifiers (UMIs), which are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, e.g., when multiplex sequencing will be used to sequence DNA from a plurality of samples (e.g., from the same or different subjects) in a single sequencing reaction, a patient-specific index is also added to the nucleic acid molecules. In some embodiments, the patient specific index is a short nucleic acid sequence (e.g., 3-20 nucleotides) that are added to ends of DNA fragments during library construction, that serve as a unique tag that can be used to identify sequence reads originating from a specific patient sample. Examples of identifier sequences are described, for example, in Kivioja et al., Nat. Methods 9 (1): 72-74 (2011) and Islam et al., Nat. Methods 11 (2): 163-66 (2014), the contents of which are hereby incorporated by reference, in their entireties, for all purposes.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the DNA molecule to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached DNA fragment. This provides a way to identify sequence reads that came from the same original fragment in downstream analysis.

In some embodiments, DNA libraries are amplified and purified using commercial reagents, (e.g., Axygen MAG PCR clean up beads). In some such embodiments, the concentration and/or quantity of the DNA molecules are then quantified using a fluorescent dye and a fluorescence microplate reader, standard spectrofluorometer, or filter fluorometer. In some embodiments, library amplification is performed on a device (e.g., an Illumina C-Bot2) and the resulting flow cell containing amplified target-captured DNA libraries is sequenced on a next generation sequencer (e.g., an Illumina HiSeq 4000 or an Illumina NovaSeq 6000) to a unique on-target depth selected by the user. In some embodiments, DNA library preparation is performed with an automated system, using a liquid handling robot (e.g., a SciClone NGSx).

In some embodiments, where feature data 125 includes methylation states 132 for one or more genomic locations, nucleic acids isolated from the biological sample (e.g., cfDNA) are treated to convert unmethylated cytosines to uracils, e.g., prior to generating the sequencing library. Accordingly, when the nucleic acids are sequenced, all cytosines called in the sequencing reaction were necessarily methylated, since the unmethylated cytosines were converted to uracils and accordingly would have been called as thymidines, rather than cytosines, in the sequencing reaction. Commercial kits are available for bisulfite-mediated conversion of methylated cytosines to uracils, for instance, the EZ DNA Methylation™-Gold, EZ DNA Methylation™-Direct, and EZ DNA Methylation™-Lightning kit (available from Zymo Research Corp (Irvine, CA)).

Commercial kits are also available for enzymatic conversion of methylated cytosines to uracils, for example, the APOBEC-Seq kit (available from NEBiolabs, Ipswich, MA). The Enzymatic methyl-seq conversion is a two-step enzymatic conversion process to detect modified cytosines. The first step uses TET2 and an oxidation enhancer to protect modified cytosines from downstream deamination. TET2 enzymatically oxidizes 5 mC and 5 hmC through a cascade reaction into 5-carboxycytosine [5-methylcytosine (5 mC) P5-hydroxymethylcytosine (5 hmC) [symbol] 5-formylcytosine (5 fC) [symbol] 5-carboxycytosine (5 caC)]. This protects 5 mC and 5 hmC from deamination. 5 hmC can also be protected from deamination by glucosylation to form 5 ghmc using the oxidation enhancer. The second enzymatic step uses APOBEC to deaminate cytosine but does not convert 5 caC and 5 ghmC.

In some embodiments, wet lab processing 204 includes pooling (308) DNA molecules from a plurality of libraries, corresponding to different samples from the same and/or different patients, to form a sequencing pool of DNA libraries. When the pool of DNA libraries is sequenced, the resulting sequence reads correspond to nucleic acids isolated from multiple samples. The sequence reads can be separated into different sequence read files, corresponding to the various samples represented in the sequencing read based on the unique identifiers present in the added nucleic acid fragments. In this fashion, a single sequencing reaction can generate sequence reads from multiple samples. Advantageously, this allows for the processing of more samples per sequencing reaction.

In some embodiments, wet lab processing 204 includes enriching (310) a sequencing library, or pool of sequencing libraries, for target nucleic acids, e.g., nucleic acids encompassing loci that are informative for precision oncology and/or used as internal controls for the sequencing or bioinformatics processes. In some embodiments, enrichment is achieved by hybridizing target nucleic acids in the sequencing library to probes that hybridize to the target sequences, and then isolating the captured nucleic acids away from off-target nucleic acids that are not bound by the capture probes.

Advantageously, enriching for target sequences prior to sequencing nucleic acids significantly reduces the costs and time associated with sequencing, facilitates multiplex sequencing by allowing multiple samples to be mixed together for a single sequencing reaction, and significantly reduces the computation burden of aligning the resulting sequence reads, as a result of significantly reducing the total amount of nucleic acids analyzed from each sample.

In some embodiments, the enrichment is performed prior to pooling multiple nucleic acid sequencing libraries. However, in other embodiments, the enrichment is performed after pooling nucleic acid sequencing libraries, which has the advantage of reducing the number of enrichment assays that have to be performed.

In some embodiments, the enrichment is performed prior to generating a nucleic acid sequencing library. This has the advantage that fewer reagents are needed to perform both the enrichment (because there are fewer target sequences at this point, prior to library amplification) and the library production (because there are fewer nucleic acid molecules to tag and amplify after the enrichment). However, this raises the possibility of pull-down bias and/or that small variations in the enrichment protocol will result in less consistent results.

In some embodiments, nucleic acid libraries are pooled (two or more DNA libraries may be mixed to create a pool) and treated with reagents to reduce off-target capture, for example Human COT-1 and/or IDT xGen Universal Blockers. Pools may be dried in a vacufuge and resuspended. DNA libraries or pools may be hybridized to a probe set (for example, a probe set specific to a panel that includes loci from at least 100, 600, 1,000, 10,000, etc. of the 19,000 known human genes) and amplified with commercially available reagents (for example, the KAPA HiFi HotStart ReadyMix). For example, in some embodiments, a pool is incubated in an incubator, PCR machine, water bath, or other temperature-modulating device to allow probes to hybridize. Pools may then be mixed with Streptavidin-coated beads or another means for capturing hybridized DNA-probe molecules, such as DNA molecules representing exons of the human genome and/or genes selected for a genetic panel.

Pools may be amplified and purified more than once using commercially available reagents, for example, the KAPA HiFi Library Amplification kit and Axygen MAG PCR clean up beads, respectively. The pools or DNA libraries may be analyzed to determine the concentration or quantity of DNA molecules, for example by using a fluorescent dye (for example, PicoGreen pool quantification) and a fluorescence microplate reader, standard spectrofluorometer, or filter fluorometer. In one example, the DNA library preparation and/or capture steps may be performed with an automated system, using a liquid handling robot (for example, a SciClone NGSx).

In some embodiments, e.g., where a whole genome sequencing method will be used, nucleic acid sequencing libraries are not target-enriched prior to sequencing, in order to obtain sequencing data on substantially all of the competent nucleic acids in the sequencing library. Similarly, in some embodiments, e.g., where a whole genome sequencing method will be used, nucleic acid sequencing libraries are not mixed, because of bandwidth limitations related to obtaining significant sequencing depth across an entire genome. However, in other embodiments, e.g., where a low pass whole genome sequencing (LPWGS) methodology will be used, nucleic acid sequencing libraries can still be pooled, because very low average sequencing coverage is achieved across a respective genome, e.g., between about 0.5× and about 5×.

In some embodiments, a plurality of nucleic acid probes (e.g., a probe set) is used to enrich one or more target sequences in a nucleic acid sample (e.g., an isolated nucleic acid sample or a nucleic acid sequencing library), e.g., where one or more target sequences is informative for precision oncology. For instance, in some embodiments, one or more of the target sequences encompasses a locus that is associated with an actionable allele. That is, variations of the target sequence are associated with targeted therapeutic approaches. In some embodiments, one or more of the target sequences and/or a property of one or more of the target sequences is used in a classifier trained to distinguish two or more cancer states.

In some embodiments, the probe set includes probes targeting one or more gene loci, e.g., exon or intron loci. In some embodiments, the probe set includes probes targeting one or more loci not encoding a protein, e.g., regulatory loci, miRNA loci, and other non-coding loci, e.g., that have been found to be associated with cancer. In some embodiments, the plurality of loci include at least 25, 50, 100, 150, 200, 250, 300, 350, 400, 500, 750, 1000, 2500, 5000, or more human genomic loci.

In some embodiments, the probe set includes probes targeting one or more of the genes listed in Table 1. In some embodiments, the probe set includes probes targeting at least 5 of the genes listed in Table 1. In some embodiments, the probe set includes probes targeting at least 10 of the genes listed in Table 1. In some embodiments, the probe set includes probes targeting at least 25 of the genes listed in Table 1. In some embodiments, the probe set includes probes targeting at least 50 of the genes listed in Table 1. In some embodiments, the probe set includes probes targeting at least 75 of the genes listed in Table 1. In some embodiments, the probe set includes probes targeting at least 100 of the genes listed in Table 1. In some embodiments, the probe set includes probes targeting all of the genes listed in Table 1.

TABLE 1
An example panel of 105 genes that are informative for precision oncology.
Liquid Biopsy Gene Panel

| | | | | | | |
|---|---|---|---|---|---|---|
| ALK | B2M | ERRFI1 | IDH2 | MSH6 | PIK3R1 | SPOP |
| FGFR2 | BAP1 | ESR1 | JAK1 | MTOR | PMS2 | STK11 |
| FGFR3 | BRCA1 | EZH2 | JAK2 | MYCN | PTCH1 | TERT |
| NTRK1 | BRCA2 | FBXW7 | JAK3 | NF1 | PTEN | TP53 |
| RET | BTK | FGFR1 | KDR | NF2 | PTPN11 | TSC1 |
| ROS1 | CCND1 | FGFR4 | KEAP1 | NFE2L2 | RAD51C | TSC2 |
| BRAF | CCND2 | FLT3 | KIT | NOTCH1 | RAF1 | UGT1A1 |
| AKT1 | CCND3 | FOXL2 | KRAS | NPM1 | RB1 | VHL |
| AKT2 | CDH1 | GATA3 | MAP2K1 | NRAS | RHEB | CCNE1 |
| APC | CDK4 | GNA11 | MAP2K2 | PALB2 | RHOA | CD274 |
| AR | CDK6 | GNAQ | MAPK1 | PBRM1 | RIT1 | EGFR |
| ARAF | CDKN2A | GNAS | MLH1 | PDCD1LG2 | RNF43 | ERBB2 |
| ARID1A | CTNNB1 | HNF1A | MPL | PDGFRA | SDHA | MET |
| ATM | DDR2 | HRAS | MSH2 | PDGFRB | SMAD4 | MYC |
| ATR | DPYD | IDH1 | MSH3 | PIK3CA | SMO | KMT2A |

Generally, probes for enrichment of nucleic acids (e.g., cfDNA obtained from a liquid biopsy sample) include DNA, RNA, or a modified nucleic acid structure with a base sequence that is complementary to a locus of interest. For instance, a probe designed to hybridize to a locus in a cfDNA molecule can contain a sequence that is complementary to either strand, because the cfDNA molecules are double stranded. In some embodiments, each probe in the plurality of probes includes a nucleic acid sequence that is identical or complementary to at least 10, at least 11, at least 12, at least 13, at least 14, or at least 15 consecutive bases of a locus of interest. In some embodiments, each probe in the plurality of probes includes a nucleic acid sequence that is identical or complementary to at least 20, 25, 30, 40, 50, 75, 100, 150, 200, or more consecutive bases of a locus of interest.

Targeted-panels provide several benefits for nucleic acid sequencing. For example, in some embodiments, algorithms for discriminating between, e.g., a first and second cancer condition can be trained on smaller, more informative data sets (e.g., fewer genes), which leads to more computationally efficient training of classifiers that discriminate between the first and second cancer states. Such improvements in computational efficiency, owing to the reduced size of the discriminating gene set, can advantageously either be used to speed up classifier training or be used to improve the performance of such classifiers (e.g., through more extensive training of the classifier).

In some embodiments, the gene panel is a whole-exome panel that analyzes the exomes of a biological sample. In some embodiments, the gene panel is a whole-genome panel that analyzes the genome of a specimen. In some preferred embodiments, the gene panel is optimized for use with liquid biopsy samples (e.g., to provide clinical decision support for solid tumors). See, for example, Table 1 above.

In some embodiments, the probes include additional nucleic acid sequences that do not share any homology to the loci of interest. For example, in some embodiments, the probes also include nucleic acid sequences containing an identifier sequence, e.g., a unique molecular identifier (UMI), e.g., that is unique to a particular sample or subject. Examples of identifier sequences are described, for example, in Kivioja et al., 2011, Nat. Methods 9(1), pp. 72-74 and Islam et al., 2014, Nat. Methods 11(2), pp. 163-66, which are incorporated by reference herein. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to a nucleic acid molecule that is complementary to the locus of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, e.g., a dip-stick or magnetic bead, for recovering the nucleic acid of interest. In some embodiments, the methods described herein include amplifying the nucleic acids that bound to the probe set prior to further analysis, e.g., sequencing. Methods for amplifying nucleic acids, e.g., by PCR, are well known in the art.

Sequence reads are then generated (312) from the sequencing library or pool of sequencing libraries. Sequencing data may be acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), nanopore sequencing (Oxford Nanopore Technologies), or paired-end sequencing. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

Next-generation sequencing produces millions of short reads (e.g., sequence reads) for each biological sample. Accordingly, in some embodiments, the plurality of sequence reads obtained by next-generation sequencing of cfDNA molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides.

In some embodiments, sequencing is performed after enriching for nucleic acids (e.g., cfDNA, gDNA, and/or RNA) encompassing a plurality of predetermined target sequences, e.g., human genes and/or non-coding sequences associated with cancer. Advantageously, sequencing a nucleic acid sample that has been enriched for target nucleic acids, rather than all nucleic acids isolated from a biological sample, significantly reduces the average time and cost of the sequencing reaction. Accordingly, in some preferred embodiments, the methods described herein include obtaining a plurality of sequence reads of nucleic acids that have been hybridized to a probe set for hybrid-capture enrichment (e.g., of one or more genes listed in Table 1).

In some embodiments, panel-targeting sequencing is performed to an average on-target depth of at least 500×, at least 750×, at least 1000×, at least 2500×, at least 500×, at least 10,000×, or greater depth. In some embodiments, samples are further assessed for uniformity above a sequencing depth threshold (e.g., 95% of all targeted base pairs at 300× sequencing depth). In some embodiments, the sequencing depth threshold is a minimum depth selected by a user or practitioner.

In some embodiments, the sequence reads are obtained by a whole genome or whole exome sequencing methodology. In some such embodiments, whole exome capture steps may be performed with an automated system, using a liquid handling robot (for example, a SciClone NGSx). Whole genome sequencing, and to some extent whole exome sequencing, is typically performed at lower sequencing depth than smaller target-panel sequencing reactions, because many more loci are being sequenced. For example, in some embodiments, whole genome or whole exome sequencing is performed to an average sequencing depth of at least 3×, at least 5×, at least 10×, at least 15×, at least 20×, or greater. In some embodiments, low-pass whole genome sequencing (LPWGS) techniques are used for whole genome or whole exome sequencing. LPWGS is typically performed to an average sequencing depth of about 0.25% to about 5%, more typically to an average sequencing depth of about 0.5× to about 3×.

Because of the differences in the sequencing methodologies, data obtained from targeted-panel sequencing is better suited for certain analyses than data obtained from whole genome/whole exome sequencing, and vice versa. For instance, because of the higher sequencing depth achieved by targeted-panel sequencing, the resulting sequence data is better suited for the identification of variant alleles present at low allelic fractions in the sample, e.g., less than 20%. By contrast, data generated from whole genome/whole exome sequencing is better suited for the estimation of genome-wide metrics, such as tumor mutational burden, because the entire genome is better represented in the sequencing data. Accordingly, in some embodiments, a nucleic acid sample, e.g., a cfDNA, gDNA, or mRNA sample, is evaluated using both targeted-panel sequencing and whole genome/whole exome sequencing (e.g., LPWGS).

In some embodiments, the raw sequence reads resulting from the sequencing reaction are output from the sequencer in a native file format, e.g., a BCL file. In some embodiments, the native file is passed directly to a bioinformatics pipeline (e.g., variant analysis 206), components of which are described in detail below. In other embodiments, one or more pre-processing steps are performed prior to passing the sequences to the bioinformatics platform. For instance, in some embodiments, the format of the sequence read file is converted from the native file format (e.g., BCL) to a file format compatible with one or more algorithms used in the bioinformatics pipeline (e.g., FASTQ or FASTA). In some embodiments, the raw sequence reads are filtered to remove sequences that do not meet one or more quality thresholds. In some embodiments, raw sequence reads generated from the same unique nucleic acid molecule in the sequencing read are collapsed into a single sequence read representing the molecule, e.g., using UMIs as described above. In some embodiments, one or more of these pre-processing steps are performed within the bioinformatics pipeline itself.

In one example, a sequencer may generate a BCL file. A BCL file may include raw image data of a plurality of patient specimens which are sequenced. BCL image data is an image of the flow cell across each cycle during sequencing. A cycle may be implemented by illuminating a patient specimen with a specific wavelength of electromagnetic radiation, generating a plurality of images which may be processed into base calls via BCL to FASTQ processing algorithms which identify which base pairs are present at each cycle. The resulting FASTQ file includes the entirety of reads for each patient specimen paired with a quality metric, e.g., in a range from 0 to 64 where a 64 is the best quality and a 0 is the worst quality. In embodiments where both a liquid biopsy sample and a normal tissue sample are sequenced, sequence reads in the corresponding FASTQ files may be matched, such that a liquid biopsy-normal analysis may be performed.

FASTQ format is a text-based format for storing both a biological sequence, such as nucleotide sequence, and its corresponding quality scores. These FASTQ files are analyzed to determine what genetic variants or copy number changes are present in the sample. Each FASTQ file contains reads that may be paired-end or single reads, and may be short-reads or long-reads, where each read represents one detected sequence of nucleotides in a nucleic acid molecule that was isolated from the patient sample or a copy of the nucleic acid molecule, detected by the sequencer. Each read in the FASTQ file is also associated with a quality rating. The quality rating may reflect the likelihood that an error occurred during the sequencing procedure that affected the associated read. In some embodiments, the results of paired-end sequencing of each isolated nucleic acid sample are contained in a split pair of FASTQ files, for efficiency. Thus, in some embodiments, forward (Read 1) and reverse (Read 2) sequences of each isolated nucleic acid sample are stored separately but in the same order and under the same identifier.

In various embodiments, the bioinformatics pipeline may filter FASTQ data from the corresponding sequence data file for each respective biological sample. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

While workflow 200 illustrates steps for obtaining a biological sample, extracting nucleic acids from the biological sample, and sequencing the isolated nucleic acids, in some embodiments, sequencing data used in the improved systems and methods described herein (e.g., which include improved methods for estimating circulating tumor fraction and cancer monitoring using low-pass whole genome methylation sequencing) is obtained by receiving previously generated sequence reads, in electronic form.

Referring again to FIG. 2A, nucleic acid sequencing data 122 generated from the one or more patient samples is then evaluated (e.g., via variant analysis 206) in a bioinformatics pipeline, e.g., using bioinformatics module 140 of system 100, to identify genomic alterations and other metrics in the cancer genome of the patient. An example overview for a bioinformatics pipeline is described below with respect to FIGS. 4A-4F. Advantageously, in some embodiments, the present disclosure improves bioinformatics pipelines, like pipeline 206, by improving for estimating circulating tumor fraction and cancer monitoring using low-pass whole genome methylation sequencing.

FIG. 4A illustrates an example bioinformatics pipeline 206 (e.g., as used for feature extraction in the workflows illustrated in FIGS. 2A and 3) for providing clinical support for precision oncology. As shown in FIG. 4A, sequencing data 122 obtained from the wet lab processing 204 (e.g., sequence reads 314) is input into the pipeline.

In various embodiments, the bioinformatics pipeline includes a circulating tumor DNA (ctDNA) pipeline for analyzing liquid biopsy samples. The pipeline may detect SNVs, INDELs, copy number amplifications/deletions and genomic rearrangements (for example, fusions). The pipeline may employ unique molecular index (UMI)-based consensus base calling as a method of error suppression as well as a Bayesian tri-nucleotide context-based position level error suppression. In various embodiments, it is able to detect variants having a 0.1%, 0.15%, 0.2%, 0.25%, 0.3%, 0.4%, or 0.5% variant allele fraction.

In some embodiments, the sequencing data is processed (e.g., using sequence data processing module 141) to prepare it for genomic feature identification 385. For instance, in some embodiments as described above, the sequencing data is present in a native file format provided by the sequencer. Accordingly, in some embodiments, the system (e.g., system 100) applies a pre-processing algorithm 142 to convert the file format (318) to one that is recognized by one or more upstream processing algorithms. For example, BCL file outputs from a sequencer can be converted to a FASTQ file format using the bel2fastq or bcl2fastq2 conversion software (Illumina®). FASTQ format is a text-based format for storing both a biological sequence, such as nucleotide sequence, and its corresponding quality scores. These FASTQ files are analyzed to determine what genetic variants, copy number changes, etc., are present in the sample.

In some embodiments, other preprocessing steps are performed, e.g., filtering sequence reads 122 based on a desired quality, e.g., size and/or quality of the base calling. In some embodiments, quality control checks are performed to ensure the data is sufficient for variant calling. For instance, entire reads, individual nucleotides, or multiple nucleotides that are likely to have errors may be discarded based on the quality rating associated with the read in the FASTQ file, the known error rate of the sequencer, and/or a comparison between each nucleotide in the read and one or more nucleotides in other reads that has been aligned to the same location in the reference genome. Filtering may be done in part or in its entirety by various software tools, for example, a software tool such as Skewer. See, Jiang, H. et al., BMC Bioinformatics 15(182):1-12 (2014). FASTQ files may be analyzed for rapid assessment of quality control and reads, for example, by a sequencing data QC software such as AfterQC, Kraken, RNA-SeQC, FastQC, or another similar software program. For paired-end reads, reads may be merged.

In some embodiments, when both a liquid biopsy sample and a normal tissue sample from the patient are sequenced, two FASTQ output files are generated, one for the liquid biopsy sample and one for the normal tissue sample. A ‘matched’ (e.g., panel-specific) workflow is run to jointly analyze the liquid biopsy-normal matched FASTQ files. When a matched normal sample is not available from the patient, FASTQ files from the liquid biopsy sample are analyzed in the ‘tumor-only’ mode. See, for example, FIG. 4B. If two or more patient samples are processed simultaneously on the same sequencer flow cell, e.g., a liquid biopsy sample and a normal tissue sample, a difference in the sequence of the adapters used for each patient sample barcodes nucleic acids extracted from both samples, to associate each read with the correct patient sample and facilitate assignment to the correct FASTQ file.

For efficiency, in some embodiments, the results of paired-end sequencing of each isolate are contained in a split pair of FASTQ files. Forward (Read 1) and reverse (Read 2) sequences of each tumor and normal isolate are stored separately but in the same order and under the same identifier. See, for example, FIG. 4C. In various embodiments, the bioinformatics pipeline may filter FASTQ data from each isolate. Such filtering may include correcting or masking sequencer errors and removing (trimming) low quality sequences or bases, adapter sequences, contaminations, chimeric reads, overrepresented sequences, biases caused by library preparation, amplification, or capture, and other errors. See, for example, FIG. 4D.

Similarly, in some embodiments, sequencing (312) is performed on a pool of nucleic acid sequencing libraries prepared from different biological samples, e.g., from the same or different patients. Accordingly, in some embodiments, the system demultiplexes (320) the data (e.g., using demultiplexing algorithm 144) to separate sequence reads into separate files for each sequencing library included in the sequencing pool, e.g., based on UMI or patient identifier sequences added to the nucleic acid fragments during sequencing library preparation, as described above. In some embodiments, the demultiplexing algorithm is part of the same software package as one or more pre-processing algorithms 142. For instance, the bcl2fastq or bcl2fastq2 conversion software (Illumina®) include instructions for both converting the native file format output from the sequencer and demultiplexing sequence reads 122 output from the reaction.

The sequence reads are then aligned (322), e.g., using an alignment algorithm 143, to a reference sequence construct 158, e.g., a reference genome, reference exome, or other reference construct prepared for a particular targeted-panel sequencing reaction. For example, in some embodiments, individual sequence reads 123, in electronic form (e.g., in FASTQ files), are aligned against a reference sequence construct for the species of the subject (e.g., a reference human genome) by identifying a sequence in a region of the reference sequence construct that best matches the sequence of nucleotides in the sequence read. In some embodiments, the sequence reads are aligned to a reference exome or reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. Any of a variety of alignment tools can be used for this task.

For instance, local sequence alignment algorithms compare subsequences of different lengths in the query sequence (e.g., sequence read) to subsequences in the subject sequence (e.g., reference construct) to create the best alignment for each portion of the query sequence. In contrast, global sequence alignment algorithms align the entirety of the sequences, e.g., end to end. Examples of local sequence alignment algorithms include the Smith-Waterman algorithm (see, for example, Smith and Waterman, J Mol. Biol., 147(1):195-97 (1981), which is incorporated herein by reference), Lalign (see, for example, Huang and Miller, Adv. Appl. Math, 12:337-57 (1991), which is incorporated by reference herein), and PatternHunter (see, for example, Ma B. et al., Bioinformatics, 18(3):440-45 (2002), which is incorporated by reference herein).

In some embodiments, the read mapping process starts by building an index of either the reference genome or the reads, which is then used to retrieve the set of positions in the reference sequence where the reads are more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6 (Suppl. 11), S6-S12, each of which is hereby incorporated by reference. In some embodiments, the mapping tools methodology makes use of a hash table or a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference.

Other software programs designed to align reads include, for example, Novoalign (Novocraft, Inc.), Bowtie, Burrows Wheeler Aligner (BWA), and/or programs that use a Smith-Waterman algorithm. Candidate reference genomes include, for example, hg19, GRCh38, hg38, GRCh37, and/or other reference genomes developed by the Genome Reference Consortium. In some embodiments, the alignment generates a SAM file, which stores the locations of the start and end of each read according to coordinates in the reference genome and the coverage (number of reads) for each nucleotide in the reference genome.

For example, in some embodiments, each read of a FASTQ file is aligned to a location in the human genome having a sequence that best matches the sequence of nucleotides in the read. There are many software programs designed to align reads, for example, Novoalign (Novocraft, Inc.), Bowtie, Burrows Wheeler Aligner (BWA), programs that use a Smith-Waterman algorithm, etc. Alignment may be directed using a reference genome (for example, hg19, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. In some embodiments, one or more SAM files are generated for the alignment, which store the locations of the start and end of each read according to coordinates in the reference genome and the coverage (number of reads) for each nucleotide in the reference genome. The SAM files may be converted to BAM files. In some embodiments, the BAM files are sorted and duplicate reads are marked for deletion, resulting in de-duplicated BAM files.

In some embodiments, adapter-trimmed FASTQ files are aligned to the 19th edition of the human reference genome build (HG19) using Burrows-Wheeler Aligner (BWA) [PMC2705234]. Following alignment, reads are grouped by alignment position and UMI family and collapsed into consensus sequences, for example, using fgbio tools (http://fulcrumgenomics.github.io/fgbio/). Bases with insufficient quality or significant disagreement among family members (for example, when it is uncertain whether the base is an adenine, cytosine, guanine, etc.) may be replaced by N's to represent a wildcard nucleotide type. PHRED scores are then scaled based on initial base calling estimates combined across all family members. Following single-strand consensus generation, duplex consensus sequences are generated by comparing the forward and reverse oriented PCR products with mirrored UMI sequences. In various embodiments, a consensus can be generated across read pairs. Otherwise, single-strand consensus calls will be used. Following consensus calling, filtering is performed to remove low-quality consensus fragments. The consensus fragments are then re-aligned to the human reference genome using BWA. A BAM output file is generated after the re-alignment, then sorted by alignment position, and indexed.

In some embodiments, where both a liquid biopsy sample and a normal tissue sample are analyzed, this process produces a liquid biopsy BAM file (e.g., Liquid BAM 124-1-i-cf) and a normal BAM file (e.g., Germline BAM 124-1-i-g), as illustrated in FIG. 4A. In various embodiments, BAM files may be analyzed to detect genetic variants and other genetic features, including single nucleotide variants (SNVs), copy number variants (CNVs), gene rearrangements, etc.

In some embodiments, the sequencing data is normalized, e.g., to account for pull-down, amplification, and/or sequencing bias (e.g., mappability, GC bias etc.). See, for example, Schwartz et al., PLOS ONE 6(1):e16685 (2011) and Benjamini and Speed, Nucleic Acids Research 40(10):e72 (2012), the contents of which are hereby incorporated by reference, in their entireties, for all purposes.

In some embodiments, SAM files generated after alignment are converted to BAM files 124. Thus, after preprocessing sequencing data generated for a pooled sequencing reaction, BAM files are generated for each of the sequencing libraries present in the master sequencing pools. For example, as illustrated in FIG. 4A, separate BAM files are generated for each of three samples acquired from subject 1 at time i (e.g., tumor BAM 124-1-i-t corresponding to alignments of sequence reads of nucleic acids isolated from a solid tumor sample from subject 1, Liquid BAM 124-1-i-cf corresponding to alignments of sequence reads of nucleic acids isolated from a liquid biopsy sample from subject 1, and Germline BAM 124-1-i-g corresponding to alignments of sequence reads of nucleic acids isolated from a normal tissue sample from subject 1), and one or more samples acquired from one or more additional subjects at time j (e.g., Tumor BAM 124-2-j-t corresponding to alignments of sequence reads of nucleic acids isolated from a solid tumor sample from subject 2). In some embodiments, BAM files are sorted, and duplicate reads are marked for deletion, resulting in de-duplicated BAM files. For example, tools like SamBAMBA mark and filter duplicate alignments in the sorted BAM files.

Many of the embodiments described below, in conjunction with FIG. 4, relate to analyses performed using sequencing data from cfDNA of a cancer patient, e.g., obtained from a liquid biopsy sample of the patient. Generally, these embodiments are independent and, thus, not reliant upon any particular sequencing data generation methods, e.g., sample preparation, sequencing, and/or data pre-processing methodologies. However, in some embodiments, the methods described below include one or more steps 204 of generating sequencing data, as illustrated in FIGS. 2A and 3.

Alignment files prepared as described above (e.g., BAM files 124) are then passed to a feature extraction module 145, where the sequences are analyzed (324) to identify genomic alterations (e.g., SNVs/MNVs, indels, genomic rearrangements, copy number variations, etc.) and/or determine various characteristics of the patient's cancer (e.g., MSI status, TMB, tumor ploidy, HRD status, tumor fraction, tumor purity, methylation patterns, etc.). Many software packages for identifying genomic alterations are known in the art, for example, freebayes, PolyBayse, samtools, GATK, pindel, SAMtools, Breakdancer, Cortex, Crest, Delly, Gridss, Hydra, Lumpy, Manta, and Socrates. For a review of many of these variant calling packages see, for example, Cameron, D. L. et al., Nat. Commun., 10(3240):1-11 (2019), the content of which is hereby incorporated by reference, in its entirety, for all purposes. Generally, these software packages identify variants in sorted SAM or BAM files 124, relative to one or more reference sequence constructs 158. The software packages then output a file e.g., a raw VCF (variant call format), listing the variants (e.g., genomic features 131) called and identifying their location relevant to the reference sequence construct (e.g., where the sequence of the sample nucleic acids differ from the corresponding sequence in the reference construct). In some embodiments, system 100 digests the contents of the native output file to populate feature data 125 in test patient data store 120. In other embodiments, the native output file serves as the record of these genomic features 131 in test patient data store 120.

Generally, the systems described herein can employ any combination of available variant calling software packages and internally developed variant identification algorithm. In some embodiments, the output of a particular algorithm of a variant calling software is further evaluated, e.g., to improve variant identification. Accordingly, in some embodiments, system 100 employs an available variant calling software package to perform some of all of the functionality of one or more of the algorithms shown in feature extraction module 145.

In some embodiments, as illustrated in FIG. 1A, separate algorithms (or the same algorithm implemented using different parameters) are applied to identify variants unique to the cancer genome of the patient and variants existing in the germline of the subject. In other embodiments, variants are identified indiscriminately and later classified as either germline or somatic, e.g., based on sequencing data, population data, or a combination thereof. In some embodiments, variants are classified as germline variants, and/or non-actionable variants, when they are represented in the population above a threshold level, e.g., as determined using a population database such as ExAC or gnomAD. For instance, in some embodiments, variants that are represented in at least 1% of the alleles in a population are annotated as germline and/or non-actionable. In other embodiments, variants that are represented in at least 2%, at least 3%, at least 4%, at least 5%, at least 7.5%, at least 10%, or more of the alleles in a population are annotated as germline and/or non-actionable. In some embodiments, sequencing data from a matched sample from the patient, e.g., a normal tissue sample, is used to annotate variants identified in a cancerous sample from the subject. That is, variants that are present in both the cancerous sample and the normal sample represent those variants that were in the germline prior to the patient developing cancer, and can be annotated as germline variants.

In various aspects, the detected genetic variants and genetic features are analyzed as a form of quality control. For example, a pattern of detected genetic variants or features may indicate an issue related to the sample, sequencing procedure, and/or bioinformatics pipeline (e.g., example, contamination of the sample, mislabeling of the sample, a change in reagents, a change in the sequencing procedure and/or bioinformatics pipeline, etc.).

FIG. 4E illustrates an example workflow for genomic feature identification (324). This particular workflow is only an example of one possible collection and arrangement of algorithms for feature extraction from sequencing data 124. Generally, any combination of the modules and algorithms of feature extraction module 145, e.g., illustrated in FIG. 1A, can be used for a bioinformatics pipeline, and particularly for a bioinformatics pipeline for analyzing liquid biopsy samples. For instance, in some embodiments, an architecture useful for the methods and systems described herein includes at least one of the modules or variant calling algorithms shown in feature extraction module 145. In some embodiments, an architecture includes at least 2, 3, 4, 5, 6, 7, 8, 9, 10, or more of the modules or variant calling algorithms shown in feature extraction module 145. Further, in some embodiments, feature extraction modules and/or algorithms not illustrated in FIG. 1A find use in the methods and systems described herein.

### Variant Identification

In some embodiments, variant analysis of aligned sequence reads, e.g., in SAM or BAM format, includes identification of single nucleotide variants (SNVs), multiple nucleotide variants (MNVs), indels (e.g., nucleotide additions and deletions), and/or genomic rearrangements (e.g., inversions, translocations, and gene fusions) using variant identification module 146, e.g., which includes a SNV/MNV calling algorithm (e.g., SNV/MNV calling algorithm 147), an indel calling algorithm (e.g., indel calling algorithm 148), and/or one or more genomic rearrangement calling algorithms (e.g., genomic rearrangement calling algorithm 149). An overview of an example method for variant identification is shown in FIG. 4E. Essentially, the module first identifies a difference between the sequence of an aligned sequence read 124 and the reference sequence to which the sequence read is aligned (e.g., an SNV/MNV, an indel, or a genomic rearrangement) and makes a record of the variant, e.g., in a variant call format (VCF) file. For instance, software packages such as freebayes and pindel are used to call variants using sorted BAM files and reference BED files as the input. For a review of variant calling packages see, for example, Cameron, D. L. et al., Nat. Commun., 10(3240):1-11 (2019). A raw VCF file (variant call format) file is output, showing the locations where the nucleotide base in the sample is not the same as the nucleotide base in that position in the reference sequence construct.

In some embodiments, as illustrated in FIG. 4E, raw VCF data is then normalized, e.g., by parsimony and left alignment. For example, software packages such as vcfbreakmulti and vt are used to normalize multi-nucleotide polymorphic variants in the raw VCF file and a variant normalized VCF file is output. See, for example, E. Garrison, “Vcflib: A C++ library for parsing and manipulating VCF files, GitHub (found online at the URL github.com/ekg/vcflib (2012), the content of which is hereby incorporated by reference, in its entirety, for all purposes. In some embodiments, a normalization algorithm is included within the architecture of a broader variant identification software package.

An algorithm is then used to annotate the variants in the (e.g., normalized) VCF file, e.g., determines the source of the variation, e.g., whether the variant is from the germline of the subject (e.g., a germline variant), a cancerous tissue (e.g., a somatic variant), a sequencing error, or of an undeterminable source. In some embodiments, an annotation algorithm is included within the architecture of a broader variant identification software package. However, in some embodiments, an external annotation algorithm is applied to (e.g., normalized) VCF data obtained from a conventional variant identification software package. The choice to use a particular annotation algorithm is well within the purview of the skilled artisan, and in some embodiments is based upon the data being annotated.

For example, in some embodiments, where both a liquid biopsy sample and a normal tissue sample of the patient are analyzed, variants identified in the normal tissue sample inform annotation of the variants in the liquid biopsy sample. In some embodiments, where a particular variant is identified in the normal tissue sample, that variant is annotated as a germline variant in the liquid biopsy sample. Similarly, in some embodiments, where a particular variant identified in the liquid biopsy sample is not identified in the normal tissue sample, the variant is annotated as a somatic variant when the variant otherwise satisfies any additional criteria placed on somatic variant calling, e.g., a threshold variant allele frequency (VAF) in the sample.

By contrast, in some embodiments, where only a liquid biopsy sample is being analyzed, the annotation algorithm relies on other characteristics of the variant in order to annotate the origin of the variant. For instance, in some embodiments, the annotation algorithm evaluates the VAF of the variant in the sample, e.g., alone or in combination with additional characteristics of the sample, e.g., tumor fraction. Accordingly, in some embodiments, where the VAF is within a first range encompassing a value that corresponds to a 1:1 distribution of variant and reference alleles in the sample, the algorithm annotates the variant as a germline variant, because it is presumably represented in cfDNA originating from both normal and cancer tissues. Similarly, in some embodiments, where the VAF is below a baseline variant threshold, the algorithm annotates the variant as undeterminable, because there is not sufficient evidence to distinguish between the possibility that the variant arose as a result of an amplification or sequencing error and the possibility that the variant originated from a cancerous tissue. Similarly, in some embodiments, where the VAF falls between the first range and the baseline variant threshold, the algorithm annotates the variant as a somatic variant.

In some embodiments, the baseline variant threshold is a value from 0.01% VAF to 0.5% VAF. In some embodiments, the baseline variant threshold is a value from 0.05% VAF to 0.35% VAF. In some embodiments, the baseline variant threshold is a value from 0.1% VAF to 0.25% VAF. In some embodiments, the baseline variant threshold is about 0.01% VAF, 0.015% VAF, 0.02% VAF, 0.025% VAF, 0.03% VAF, 0.035% VAF, 0.04% VAF, 0.045% VAF, 0.05% VAF, 0.06% VAF, 0.07% VAF, 0.075% VAF, 0.08% VAF, 0.09% VAF, 0.1% VAF, 0.15% VAF, 0.2% VAF, 0.25% VAF, 0.3% VAF, 0.35% VAF, 0.4% VAF, 0.45% VAF, 0.5% VAF, or greater. In some embodiments, the baseline variant threshold is different for variants located in a first region, e.g., a region identified as a mutational hotspot and/or having high genomic complexity, than for variants located in a second region, e.g., a region that is not identified as a mutational hotspot and/or having average genomic complexity. For example, in some embodiments, the baseline variant threshold is a value from 0.01% to 0.25% for variants located in the first region and is a value from 0.1% to 0.5% for variants located in the second region.

In some embodiments, the first region is a region of interest in the genome that may have been manually selected based on criteria (for example, selection may be based on a known likelihood that a region is associated with variants) and the second region is a region that did not meet the selection criteria. In some embodiments, the baseline variant threshold is a value from 0.01% to 0.5% for variants located in the first region and is a value from 1% to 5% for variants located in the second region. In some embodiments, the first region is a region of interest in the genome that may have been manually selected based on criteria (for example, selection may be based on a known likelihood that a region is associated with variants) and the second region is a region selected based on a second set of criteria.

In some embodiments, a baseline variant threshold is influenced by the sequencing depth of the reaction, e.g., a locus-specific sequencing depth and/or an average sequencing depth (e.g., across a targeted panel and/or complete reference sequence construct). In some embodiments, the baseline variant threshold is dependent upon the type of variant being detected. For example, in some embodiments, different baseline variant thresholds are set for SNPs/MNVs than for indels and/or genomic rearrangements. For instance, while an apparent SNP may be introduced by amplification and/or sequencing errors, it is much less likely that a genomic rearrangement is introduced this way and, thus, a lower baseline variant threshold may be appropriate for genomic rearrangements than for SNPs/MNVs.

In some embodiments, one or more additional criteria are required to be satisfied before a variant can be annotated as a somatic variant. For instance, in some embodiments, a threshold number of unique sequence reads encompassing the variant must be present to annotate the variant as somatic. In some embodiments, the threshold number of unique sequence reads is 2, 3, 4, 5, 7, 10, 12, 15, or greater. In some embodiments, the threshold number of unique sequence reads is only applied when certain conditions are met, e.g., when the variant allele is located in a region of a certain genomic complexity. In some embodiments, the certain genomic complexity is a low genomic complexity. In some embodiments, the certain genomic complexity is an average genomic complexity. In some embodiments, the certain genomic complexity is a high genomic complexity.

In some embodiments, a threshold sequencing coverage, e.g., a locus-specific and/or an average sequencing depth (e.g., across a targeted panel and/or complete reference sequence construct) must be satisfied to annotate the variant as somatic. In some embodiments, the threshold sequencing coverage is 50×, 100×, 150×, 200×, 250×, 300×, 350×, 400× or greater. In some embodiments, the variant is located in a microsatellite instable (MSI) region. In some embodiments, the variant is not located in a microsatellite instable (MSI) region. In some embodiments, the variant has sufficient signal-to-noise ratio.

In some embodiments, bases contributing to the variant satisfy a threshold mapping quality to annotate the variant as somatic. In some embodiments, alignments contributing to the variant must satisfy a threshold alignment quality to annotate the variant as somatic. In some embodiments, a threshold value is determined for a variant detected in a somatic (cancer) sample by analyzing the threshold metric (for example, the baseline variant threshold is determined by analyzing VAF, or the threshold sequencing coverage is determined by analyzing coverage) associated with that variant in a group of germline (normal) samples that were each processed by the same sample processing and sequencing protocol as the somatic sample (process-matched). This may be used to ensure the variants are not caused by observed artifact generating processes.

In some embodiments, the threshold value is set above the median base fraction of the threshold metric value associated with the variant in more than a specified percentage of process-matched germline samples, e.g., 1, 2, 3, 4, 5, 6, 7, 8, 9, 10, or more standard deviations above the median base fraction of the threshold metric value associated with 25%, 30, 40, 50, 60, 70, 75, or more of the processed-matched germline samples. For example, in one embodiment, the threshold value is set to a value 5 standard deviations above the median base fraction of the threshold metric value associated with the variant in more than 50% of the process matched germline samples.

In some embodiments, variants around homopolymer and multimer regions known to generate artifacts may be specifically filtered to avoid such artifacts. For example, in some embodiments, strand specific filtering is performed in the direction of the read in order to minimize stranded artifacts. Similarly, in some embodiments, variants that do not exceed the stranded minimum deviation for their specific locus within a known artifact-generating region may be filtered to avoid artifacts.

Variants may be filtered using dynamic methods, such as through the application of Bayes' Theorem through a likelihood ratio test. The dynamic threshold may be based on, for example, factors such as sample specific error rate, the error rate from a healthy reference pool, and information from internal human solid tumors. Accordingly, in some embodiments, the dynamic filtering method employs a tri-nucleotide context-based Bayesian model. That is, in some embodiments, the threshold for filtering any particular putative variant is dynamically calibrated using a context-based Bayesian model that considers one or more of a sample-specific sequencing error rate, a process-matched control sequencing error rate, and/or a variant-specific frequency (e.g., determined from similar cancers). In this fashion, a minimum number of alternative alleles required to positively identify a true variant is determined for individual alleles and/or loci.

In some embodiments, certain variants pre-identified on a whitelist may be rescued, i.e., not filtered out, when they fail to pass selective filters, e.g., MSI/SN, a Bayesian filtering method, and/or a coverage, VAF or region-based filter. The rationale for whitelisting a variant is to apply less stringent filtering criteria to such a variant so that it can be reviewed and/or reported. In some embodiments, one or more variant on the whitelist is a common pathogenic variant, e.g., with high clinical relevance. In this fashion, when a variant on the whitelist fails to pass certain filters, it will be rescued and not filtered out. As used herein, MSI/SN refers to a variant filter for filtering out potential artifactual variants based on the MSI (microsatellite instable) and SN (signal-to-noise ratio) values calculated by the variant caller VarDict. See, for example, VarDict documentation, available on the internet at github.com/AstraZeneca-NGS/VarDictJava.

In some embodiments, one or more locus and/or genomic region is blacklisted, preventing somatic variant annotation for variants identified at the locus or region. In some embodiments, the variant has a length of 120, 100, 80, 60, 40, 20, 10, 5 or less base pairs. In various embodiments, any combination of the additional criteria, as well as additional criteria not listed above, may be applied to the variant calling process. Again, in some embodiments, different criteria are applied to the annotation of different types of variants.

In some embodiments, liquid biopsy assays are used to detect variant alterations present at low circulating fractions in the patient's blood. In such circumstances, it may be warranted to lower the requirements for positively identifying a variant. That is, in some embodiments, low levels of support may be sufficient to call a variant, dependent upon the reason for using the liquid biopsy assay.

In some embodiments, SNV/INDEL detection is accomplished using VarDict [PMC4914105]. Both SNVs and INDELs are called and then sorted, deduplicated, normalized and annotated. The annotation step uses SnpEff to add transcript information, 1000 genomes minor allele frequencies, COSMIC reference names and counts, ExAC allele frequencies, and Kaviar population allele frequencies. The annotated variants are then classified as germline, somatic, or uncertain using a Bayesian model based on prior expectations informed by databases of germline and cancer variants. In some embodiments, uncertain variants are treated as somatic for filtering and reporting purposes.

In some embodiments, genomic rearrangements (e.g., inversions, translocations, and gene fusions) are detected following de-multiplexing by aligning tumor FASTQ files against a human reference genome using a local alignment algorithm, such as BWA. In some embodiments, DNA reads are sorted and duplicates may be marked with a software, for example, SAMBlaster. Discordant and split reads may be further identified and separated. These data may be read into a software, for example, LUMPY, for structural variant detection. In some embodiments, structural alterations are grouped by type, recurrence, and presence and stored within a database and displayed through a fusion viewer software tool. The fusion viewer software tool may reference a database, for example, Ensembl, to determine the gene and proximal exons surrounding the breakpoint for any possible transcript generated across the breakpoint. The fusion viewer tool may then place the breakpoint 5′ or 3′ to the subsequent exon in the direction of transcription. For inversions, this orientation may be reversed for the inverted gene. After positioning of the breakpoint, the translated amino acid sequences may be generated for both genes in the chimeric protein, and a plot may be generated containing the remaining functional domains for each protein, as returned from a database, for example, Uniprot.

For instance, in an example implementation, gene rearrangements are detected using the SpeedSeq analysis pipeline. Chiang et al., 2015, “SpeedSeq: ultra-fast personal genome analysis and interpretation,” Nat Methods, (12), pg. 966. Briefly, FASTQ files are aligned to hg19 using BWA. Split reads mapped to multiple positions and read pairs mapped to discordant positions are identified and separated, then utilized to detect gene rearrangements by LUMPY. Layer et al., 2014, “I. M. LUMPY: a probabilistic framework for structural variant discovery,” Genome Biol, (15), pg. 84. Fusions can then be filtered according to the number of supporting reads.

In some embodiments, putative fusion variants supported by fewer than a minimum number of unique sequence reads are filtered. In some embodiments, the minimum number of unique sequence reads is 2, 3, 4, 5, 6, 7, 8, 9, 10, 12, 15, or 20 unique sequence reads.

### Allelic Fraction Determination

In some embodiments, the analysis of aligned sequence reads, e.g., in SAM or BAM format, includes determination of variant allele fractions (133) for one or more of the variant alleles 132 identified as described above. In some embodiments, a variant allele fraction module 151 tallies the instances that each allele is represented by a unique sequence read encompassing the variant locus of interest, generating a count for each allele represented at that locus. In some embodiments, these tallies are used to determine the ratio of the variant allele, e.g., an allele other than the most prevalent allele in the subject's population for a respective locus, to a reference allele. This variant allele fraction 133 can be used in several places in the feature extraction 206 workflow. For instance, in some embodiments, a variant allele fraction is used during annotations of identified variants, e.g., when determining whether the allele originated from a germline cell or a somatic cell. In other instances, a variant allele fraction is used in a process for estimating a tumor fraction for a liquid biopsy sample or a tumor purity for a solid tumor fraction. For instance, variant allele fractions for a plurality of somatic alleles can be used to estimate the percentage of sequence reads originating from one copy of a cancerous chromosome. Assuming a 100% tumor purity and that each cancer cell carries one copy of the variant allele, the overall purity of the tumor can be estimated. This estimate, of course, can be further corrected based on other information extracted from the sequencing data, such as copy number alterations, tumor ploidy aberrations, tumor heterozygosity, etc.

### Methylation Determination

In some embodiments, where nucleic acid sequencing library was processed by methylation sequencing, such as bi-sulfite treatment or enzymatic methyl-cytosine conversion, as described above, the analysis of aligned sequence reads, e.g., in SAM or BAM format, includes determination of methylation states 132 for one or more loci in the genome of the patient. In some embodiments, methylation sequencing data is aligned to a reference sequence construct 158 in a different fashion than non-methylation sequencing, because non-methylated cytosines are converted to uracils, and the resulting uracils are ultimately sequenced as thymines, whereas methylated cytosine are not converted to uracils and are sequenced as cytosine. Different approaches, therefore, have to be used to align these modified sequences to a reference sequence construct, such as seeding alignments with shorter regions of identity or converting all cytosines to thymidines in the sequencing data and then aligning the data to reference sequence constructs for both the plus and minus strand of the sequence construct. For review of these approaches, see Zhou Q. et al., BMC Bioinformatics, 20(47):1-11 (2019), the content of which is hereby incorporated by reference, in its entirety, for all purposes. Algorithms for calling methylated bases are known in the art. For example, Bismark is able to distinguish between cytosines in CpG, CHG, and CHH contexts. Krueger F. and Andrews S R, Bioinformatics, 27(11):1571-71 (2011), the content of which is hereby incorporated by reference, in its entirety, for all purposes.

### Copy Number Variation Analysis:

In some embodiments, the analysis of aligned sequence reads, e.g., in SAM or BAM format, includes determination of the copy number 135 for one or more locus, using a copy number variation analysis module 153. In some embodiments, where both a liquid biopsy sample and a normal tissue sample of the patient are analyzed, de-duplicated BAM files and a VCF generated from the variant calling pipeline are used to compute read depth and variation in heterozygous germline SNVs between sequencing reads for each sample. By contrast, in some embodiments, where only a liquid biopsy sample is being analyzed, comparison between a tumor sample and a pool of process-matched normal controls is used. In some embodiments, copy number analysis includes application of a circular binary segmentation algorithm and selection of segments with highly differential log 2 ratios between the cancer sample and its comparator (e.g., a matched normal or normal pool). In some embodiments, approximate integer copy number is assessed from a combination of differential coverage in segmented regions and an estimate of stromal admixture (for example, tumor purity, or the portion of a sample that is cancerous vs. non-cancerous, such as a tumor fraction for a liquid biopsy sample) is generated by analysis of heterozygous germline SNVs.

For instance, in an example implementation, copy number variants (CNVs) are analyzed using the CNVkit package. Talevich et al., PLOS Comput Biol, 12:1004873 (2016), the content of which is hereby incorporated by reference, in its entirety, for all purposes. CNVkit is used for genomic region binning, coverage calculation, bias correction, normalization to a reference pool, segmentation and visualization. The log 2 ratios between the tumor sample and a pool of process matched healthy samples from the CNVkit output are then annotated and filtered using statistical models whereby the amplification status (amplified or not-amplified) of each gene is predicted and non-focal amplifications are removed.

In some embodiments, copy number variations (CNVs) are analyzed using a combination of an open-source tool, such as CNVkit, and an annotation/filtering algorithm, e.g., implemented via a python script. CNVkit is used initially to perform genomic region binning, coverage calculation, bias correction, normalization to a reference pool, segmentation and, optionally, visualization. The bin-level copy ratios and segment-level copy ratios, in addition to their corresponding confidence intervals, from the CNVkit output are then used in the annotation and filtering step where the copy number state (amplified, neutral, deleted) of each segment and bin are determined and non-focal amplifications/deletions are filtered out based on a set of acceptance criteria. In some embodiments, one or more copy number variations selected from amplifications in the MET, EGFR, ERBB2, CD274, CCNE1, and MYC genes, and deletions in the BRCA1 and BRCA2 genes are analyzed. However, the methods described herein is not limited to only these reportable genes.

In some embodiments, CNV analysis is performed using a tumor BAM file, a target region BED file, a pool of process matched normal samples, and inputs for initial reference pool construction. Inputs for initial reference pool construction include one or more of normal BAM files, a human reference genome file, mappable regions of the genome, and a blacklist that contains recurrent problematic areas of the genome.

CNVkit utilizes both targeted captured sequencing reads and non-specifically captured off-target reads to infer copy number information. The targeted genomic region specified in the probe target BED file are divided to target bins with an average size of, e.g., 100 base pairs, which can be specified by the user. The genomic regions between the target regions, e.g., excluding regions that cannot be mapped reliably, are automatically divided into off-target (also referred to as anti-target) bins with an average size of, e.g., 150 kbp, which again can be specified by the user. Raw log 2-transformed depths are then calculated from the alignments in the input BAM file and written to two tab-delimited.cnn files, one for each of the target and off-target bins.

A pooled reference is constructed from a panel of process matched normal samples. The raw log 2 depths of target and off-target bins in each normal sample are computed as described above, and then each are median-centered and corrected for bias including GC content, genome sequence repetitiveness, target size, and/or spacing. The corrected target and off-target log 2 depths are combined, and a weighted average and spread are calculated as Tukey's biweight location and midvariance in each bin. These values are written to a tab delimited reference.cnn file, which is used to normalize an input tumor sample as follows.

The raw log 2 depths of an input sample are median-centered and bias-corrected as described in the reference construction. The corrected log 2 depth of each bin is then subtracted by the corresponding log 2 depth in the reference file, resulting in the log 2 copy ratios (also referred to as copy ratios or log 2 ratios) between the input tumor sample and the reference pool. These values are written to a tab-delimited.cnr file.

The copy ratios are then segmented, e.g., via a circular binary segmentation (CBS) algorithm or another suitable segmentation algorithm, whereby adjacent bins are grouped to larger genomic regions (segments) of equal copy number. The segment's copy ratio is calculated as the weighted mean of all bins within the segment. The confidence interval of the segment mean is estimated by bootstrapping the bin-level copy ratios within the segment. The segments' genomic ranges, copy ratios and confidence intervals are written to a tab-delimited.cns file.

### Microsatellite Instability (MSI):

In some embodiments, analysis of aligned sequence reads, e.g., in SAM or BAM format, includes analysis of the microsatellite instability status 137 of a cancer, using a microsatellite instability analysis module 154. In some embodiments, an MSI classification algorithm classifies a cancer into three categories: microsatellite instability-high (MSI-H), microsatellite stable (MSS), or microsatellite equivocal (MSE). Microsatellite instability is a clinically actionable genomic indication for cancer immunotherapy. In microsatellite instability-high (MSI-H) tumors, defects in DNA mismatch repair (MMR) can cause a hypermutated phenotype where alterations accumulate in the repetitive microsatellite regions of DNA. MSI detection is conventionally performed by subjecting tumor tissue (“solid biopsy”) to clinical next-generation sequencing or specific assays, such as MMR IHC or MSI PCR.

For example, microsatellite instability status can be assessed by determining the number of repeating units present at a plurality of microsatellite loci, e.g., 5, 10, 15, 20, 25, 30, 40, 50, 75, 100, 250, 500, 750, 1000, 2500, 5000, or more loci. In some embodiments, only reads encompassing a microsatellite locus that include a significant number of flanking nucleotides on both ends, e.g., at least 5, 10, 15, or more nucleotides flanking each end, are used for the analysis in order to avoid using reads that do not completely cover the locus. In some embodiments, a minimal number of reads, e.g., at least 5, 10, 20, 30, 40, 50, or more reads have to meet this criteria in order to use a particular microsatellite locus, in order to ensure the accuracy of the determination given the high incidence of polymerase slipping during replication of these repeated sequences.

In some embodiments, each locus is tested individually for instability, e.g., as measured by a change or variance in the number of nucleotide base repeats, e.g., in cancer-derived nucleotide sequences relative to a normal sample or standard, for example, using the Kolmogorov-Smirnov test. For example, if p≤0.05, the locus is considered unstable. The proportion of unstable microsatellite loci may be fed into a logistic regression classifier trained on samples from various cancer types, especially cancer types which have clinically determined MSI statuses, for example, colorectal and endometrial cohorts. For MSI testing where only a liquid biopsy sample is analyzed, the mean and variance for the number of repeats may be calculated for each microsatellite locus. A vector containing the mean and variance data may be put into a classifier (e.g., a support vector machine classification algorithm) trained to provide a probability that the patient is MSI-H, which may be compared to a threshold value. In some embodiments, the threshold value for calling the patient as MSI-H is at least 60% probability, or at least 65% probability, 70% probability, 75% probability, 80% probability, or greater. In some embodiments, a baseline threshold may be established to call the patient as MSS. In some embodiments, the baseline threshold is no more than 40%, or no more than 35% probability, 30% probability, 25% probability, 20% probability, or less. In some embodiments, when the output of the classifier falls within the range between the MSI-H and MSS thresholds, the patient is identified as MSE.

Other methods for determining the MSI status of a subject are known in the art. For example, in some embodiments, microsatellite instability analysis module 154 employs an MSI evaluation methods described in U.S. Provisional Patent Application Ser. No. 62/881,845, filed Aug. 1, 2019, or U.S. Provisional Application Ser. No. 62/931,600, filed Nov. 6, 2019, the contents of which are hereby incorporated by reference, in their entireties, for all purposes.

### Tumor Mutational Burden (TMB):

In some embodiments, the analysis of aligned sequence reads, e.g., in SAM or BAM format, includes determination of a mutation burden for the cancer (e.g., a tumor mutational burden 136), using a tumor mutational burden analysis module 155. Generally, a tumor mutational burden is a measure of the mutations in a cancer per unit of the patient's genome. For example, a tumor mutational burden may be expressed as a measure of central tendency (e.g., an average) of the number of somatic variants per million base pairs in the genome. In some embodiments, a tumor mutational burden refers to only a set of possible mutations, e.g., one or more of SNVs, MNVs, indels, or genomic rearrangements. In some embodiments, a tumor mutational burden refers to only a subset of one or more types of possible mutations, e.g., non-synonymous mutations, meaning those mutations that alter the amino acid sequence of an encoded protein. In other embodiments, for example, a tumor mutational burden refers to the number of one or more types of mutations that occur in protein coding sequences, e.g., regardless of whether they change the amino acid sequence of the encoded protein.

As an example, in some embodiments, a tumor mutational burden (TMB) is calculated by dividing the number of mutations (e.g., all variants or non-synonymous variants) identified in the sequencing data (e.g., as represented in a VCF file) by the size (e.g., in megabases) of a capture probe panel used for targeted sequencing. In some embodiments, a variant is included in tumor mutation burden calculation only when certain criteria are met. For instance, in some embodiments, a threshold sequence coverage for the locus associated with the variant must be met before the variant is included in the calculation, e.g., at least 25×, 50×, 75×, 100×, 250×, 500×, or greater. Similarly, in some embodiments, a minimum number of unique sequence reads encompassing the variant allele must be identified in the sequencing data, e.g., at least 4, 5, 6, 7, 8, 9, 10, or more unique sequence reads. In some embodiments, a threshold variant allelic fraction threshold must be satisfied before the variant is included in the calculation, e.g., at least 0.01%, 0.1%, 0.25%, 0.5%, 0.75%, 1%, 1.5%, 2%, 2.5%, 3%, 4%, 5%, or greater. In some embodiments, an inclusion criteria may be different for different types of variants and/or different variants of the same type. For instance, a variant detected in a mutation hotspot within the genome may face less rigorous criteria than a variant detected in a more stable locus within the genome.

Other methods for calculating tumor mutation burden in liquid biopsy samples and/or solid tissue samples are known in the art. See, for example, Fenizia F. et al., Transl Lung Cancer Res., 7(6):668-77 (2018) and Georgiadis A. et al., Clin. Cancer Res., 25(23):7024-34 (2019), the disclosures of which are hereby incorporated by reference, in their entireties, for all purposes.

### Homologous Recombination Status (HRD):

In some embodiments, analysis of aligned sequence reads, e.g., in SAM or BAM format, includes analysis of whether the cancer is homologous recombination deficient (HRD status 137-3), using a homologous recombination pathway analysis module 157.

Homologous recombination (HR) is a normal, highly conserved DNA repair process that enables the exchange of genetic information between identical or closely related DNA molecules. It is most widely used by cells to accurately repair harmful breaks (i.e. damage) that occur on both strands of DNA. DNA damage may occur from exogenous (external) sources like UV light, radiation, or chemical damage; or from endogenous (internal) sources like errors in DNA replication or other cellular processes that create DNA damage. Double strand breaks are a type of DNA damage. Using poly (ADP-ribose) polymerase (PARP) inhibitors in patients with HRD compromises two pathways of DNA repair, resulting in cell death (apoptosis). The efficacy of PARP inhibitors is improved not only in ovarian cancers displaying germline or somatic BRCA mutations, but also in cancers in which HRD is caused by other underlying etiologies.

In some embodiments, HRD status can be determined by inputting features correlated with HRD status into a classifier trained against trained to distinguish between cancers with homologous recombination pathway deficiencies and cancers without homologous recombination pathway deficiencies. For example, in some embodiments, the features include one or more of (i) a heterozygosity status for a first plurality of DNA damage repair genes in the genome of the cancerous tissue of the subject, (ii) a measure of the loss of heterozygosity across the genome of the cancerous tissue of the subject, (iii) a measure of variant alleles detected in a second plurality of DNA damage repair genes in the genome of the cancerous tissue of the subject, and (iv) a measure of variant alleles detected in the second plurality of DNA damage repair genes in the genome of the non-cancerous tissue of the subject. In some embodiments, all four of the features described above are used as features in an HRD classifier. More details about HRD classifiers using these and other features are described in U.S. patent application Ser. No. 16/789,363, filed Feb. 12, 2020, the content of which is hereby incorporated by reference, in its entirety, for all purposes.

### Quality Control

In some embodiments, a positive sensitivity control sample is processed and sequenced along with one or more clinical samples. In some embodiments, the control sample is included in at least one flow cell of a multi-flow cell reaction and is processed and sequenced each time a set of samples is sequenced or periodically throughout the course of a plurality of sets of samples. In some embodiments, the control includes a pool of controls. In some embodiments, a quality control step requires that read metrics of variants present in the control sample fall within acceptable criteria. In some embodiments, a quality control requires approval by a pathologist before the results are reported.

In some embodiments, the quality control system includes methods that pass samples for reporting if various criteria are met. Similarly, in some embodiments, the system includes methods that allow for more manual review if a sample does not meet the criteria established for automatic pass. In some embodiments, the criteria for pass of panel sequencing results include one or more of the following:
A criterion for the on-target rate of the sequencing reaction, defined as a comparison (e.g., a ratio) of (i) the number of sequenced nucleotides or reads falling within the targeted panel region of a genome and (ii) the number of sequenced nucleotides or reads falling outside of the targeted panel region of the genome. Generally, an on-target rate threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a minimum on-target rate threshold of at least 30%, at least 40%, at least 50%, at least 60%, at least 70%, or greater. In some embodiments, the on-target rate criteria is implemented as a range of acceptable on-target rates, e.g., requiring that the on-target rate for a reaction is from 30% to 70%, from 30% to 80%, from 40% to 70%, from 40% to 80%, and the like.
A criterion for the number of total reads generated by the sequencing reaction, including both unique sequence reads and non-unique sequence reads. Generally, a total read number threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a minimum number of total reads threshold of at least 100 million, 110 million, 120 million, 130 million, 140 million, 150 million, 160 million, 170 million, 180 million, 190 million, 200 million, or more total sequence reads. In some embodiments, the criterion is implemented as a range of acceptable number of total reads, e.g., requiring that the sequencing reaction generate from 50 million to 300 million total sequence reads, from 100 million to 300 million sequence reads, from 100 million to 200 million sequence reads, and the like.
A criterion for the number of unique reads generated by the sequencing reaction. Generally, a unique read number threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a minimum number of total reads threshold of at least 3 million, 4 million, 5 million, 6 million, 7 million, 8 million, 9 million, or more unique sequence reads. In some embodiments, the criterion is implemented as a range of acceptable number of unique reads, e.g., requiring that the sequencing reaction generate from 2 million to 10 million total sequence reads, from 3 million to 9 million sequence reads, from 3 million to 9 million sequence reads, and the like.
A criterion for unique read depth across the panel, defined as a measure of central tendency (e.g., a mean or median) for a distribution of the number of unique reads in the sequencing reaction encompassing the genomic regions targeted by each probe. For instance, in some embodiments, an average unique read depth is calculated for each targeted region defined in a target region BED file, using a first calculation of the number of reads mapped to the region multiplied by the read length, divided by the length of the region, if the length of the region is longer than the read length, or otherwise using a second calculation of the number of reads falling within the region multiplied by the read length. The median of unique read depth across the panel is then calculated as the median of those average unique read depths of all targeted regions. In some embodiments, the resolution as to how depth is calculated is increased or decreased, e.g., in cases where it is necessary or desirable to calculate depth for each base, or for a single gene. Generally, a unique read depth threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a minimum unique read depth threshold of at least 1500, 1750, 2000, 2250, 2500, 2750, 3000, 3250, 3500, or higher unique read depth. In some embodiments, the criterion is implemented as a range of acceptable unique read depth, e.g., requiring that the sequencing reaction generate a unique read depth of from 1000 to 4000, from 1500 to 4000, from 1500 to 4000, and the like.
A criterion for the unique read depth of a lowest percentile across the panel, defined as a measure of central tendency (e.g., a mean or median) for a distribution of the number of unique reads in the sequencing reaction encompassing the genomic regions targeted by each probe that fall within the lowest percentile of genomic regions by read depth (e.g., the first, second, third, fourth, fifth, tenth, fifteenth, twentieth, twenty-fifth, or similar percentile). Generally, a unique read depth at a lowest percentile threshold will be selected based on the sequencing technology used, the size of the targeted panel, the lowest percentile selected, and the expected number of sequence reads generated by the combination of the technology and targeted panel used. For example, in some embodiments where next generation sequencing-by-synthesis technology is used, the criterion is implemented as a minimum unique read depth threshold at the fifth percentile of at least 500, 750, 1000, 1250, 1500, 1750, 2000, 2250, 2500, or higher unique read depth. In some embodiments, the criterion is implemented as a range of acceptable unique read depth at the fifth percentile, e.g., requiring that the sequencing reaction generate a unique read depth at the fifth percentile of from 250 to 3000, from 500 to 3000, from 500 to 2500, and the like.
A criterion for the deamination or OxoG Q-score of a sequencing reaction, defined as a Q-score for the occurrence of artifacts arising from template oxidation/deamination. Generally, a deamination or OxoG Q-score threshold will be selected based on the sequencing technology used. For example, in some embodiments where next generation sequencing-by-synthesis technology is used, the criterion is implemented as a minimum deamination or OxoG Q-score threshold of at least 10, 20, 30, 40, 5,0 6,0 70, 80, 90, or higher. In some embodiments, the criterion is implemented as a range of acceptable deamination or OxoG Q-scores, e.g., from 10 to 100, from 10 to 90, and the like.
A criterion for the estimated contamination fraction is of a sequencing reaction, defined as an estimate of the fraction of template fragments in the sample being sequenced arising from contamination of the sample, commonly expressed as a decimal, e.g., where 1% contamination is expressed as 0.01. An example method for estimating contamination in a sequencing method is described in Jun G. et al., Am. J. Hum. Genet., 91:839-48 (2012). For example, in some embodiments, the criterion is implemented as a maximum contamination fraction threshold of no more than 0.001, 0.0015, 0.002, 0.0025, 0.003, 0.0035, 0.004. In some embodiments, the criterion is implemented as a range of acceptable contamination fractions, e.g., from 0.0005 to 0.005, from 0.0005 to 0.004, from 0.001 to 0.004, and the like.
A criterion for the fingerprint correlation score of a sequencing reaction, defined as a Pearson correlation coefficient calculated between the variant allele fractions of a set of pre-defined single nucleotide polymorphisms (SNPs) in two samples. An example method for determining a fingerprint correlation score is described in Sejoon L. et al., Nucleic Acids Research, Volume 45, Issue 11, 20 Jun. 2017, Page e103, the content of which is incorporated herein by reference, in its entirety, for all purposes. For example, in some embodiments, the criterion is implemented as a minimum fingerprint correlation score threshold of at least 0.1, 0.2, 0.3, 0.4, 0.5, 0.6, 0.7, 0.8, 0.9, or higher. In some embodiments, the criterion is implemented as a range of acceptable fingerprint correlation scores, e.g., from 0.1 to 0.9, from 0.2 to 0.9, from 0.3 to 0.9, and the like.
A criterion for the raw coverage of a minimum percentage of the genomic regions targeted by a probe, defined as a minimum number of unique reads in the sequencing reaction encompassing each of a minimum percentage (e.g., at least 80%, 85%, 90%, 95%, 98%, 99%, 99.5%, 99.9%, and the like) of the genomic regions targeted by the probe panel. In some embodiments, the term “unique read depth” is used to distinguish deduplicated reads from raw reads that may contain multiple reads sequenced from the same original DNA molecule via PCR. Generally, a raw coverage of a minimum percentage of the genomic regions targeted by a probe threshold will be selected based on the sequencing technology used, the size of the targeted panel, the minimum percentage selected, and the expected number of sequence reads generated by the combination of the technology and targeted panel used. For example, in some embodiments where next generation sequencing-by-synthesis technology is used, the criterion is implemented as a raw coverage of 95% of the genomic regions targeted by a probe threshold of at least 500, 750, 1000, 1250, 1500, 1750, 2000, 2250, 2500, or higher unique read depth. In some embodiments, the criterion is implemented as a range of acceptable unique read depth for 95% of the genomic regions targeted by a probe, e.g., requiring that the sequencing reaction generate a unique read depth for 95% of the targeted regions of from 250 to 3000, from 500 to 3000, from 500 to 2500, and the like.
A criterion for the PCR duplication rate of a sequencing reaction, defined as the percentage of sequence reads that arise from the same template molecule as at least one other sequence read generated by the reaction. Generally, a PCR duplication rate threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a minimum PCR duplication rate threshold of at least 91%, 92%,93%, 94%, 95%, 96%, 97%, 98%, 99%, or higher. In some embodiments, the criterion is implemented as a range of acceptable PCR duplication rates, e.g., from 90% to 100%, from 90% to 99%, from 91% to 99%, and the like.

Similarly, in some embodiments, the quality control system includes methods that fail samples for reporting if various criteria are met. In some embodiments, the system includes methods that allow for more manual review if a sample does meet the criteria established for automatic fail. In some embodiments, the criteria for failing panel sequencing results include one or more of the following:
A criterion for the on-target rate of the sequencing reaction, defined as a comparison (e.g., a ratio) of (i) the number of sequenced nucleotides or reads falling within the targeted panel region of a genome and (ii) the number of sequenced nucleotides or reads falling outside of the targeted panel region of the genome. Generally, an on-target rate threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a maximum on-target rate threshold of no more than 30%, 40%, 50%, 60%, 70%, or greater. That is, the criterion for failing the sample is satisfied when the on-target rate for the sequencing reaction is below the maximum on-target rate threshold. In some embodiments, the on-target rate criteria is implemented as not falling within a range of acceptable on-target rates, e.g., falling outside of an on-target rate for a reaction of from 30% to 70%, from 30% to 80%, from 40% to 70%, from 40% to 80%, and the like.
A criterion for the number of total reads generated by the sequencing reaction, including both unique sequence reads and non-unique sequence reads. Generally, a total read number threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a maximum number of total reads threshold of no more than 100 million, 110 million, 120 million, 130 million, 140 million, 150 million, 160 million, 170 million, 180 million, 190 million, 200 million, or more total sequence reads. That is, the criterion for failing the sample is satisfied when the number of total reads for the sequencing reaction is below the maximum total read threshold. In some embodiments, the criterion is implemented as not falling within a range of acceptable number of total reads, e.g., falling outside of a range of from 50 million to 300 million total sequence reads, from 100 million to 300 million sequence reads, from 100 million to 200 million sequence reads, and the like.
A criterion for the number of unique reads generated by the sequencing reaction. Generally, a unique read number threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a maximum number of total reads threshold of no more than 3 million, 4 million, 5 million, 6 million, 7 million, 8 million, 9 million, or more unique sequence reads. That is, the criterion for failing the sample is satisfied when the number of unique reads for the sequencing reaction is below the maximum total read threshold. In some embodiments, the criterion is implemented as not falling within a range of acceptable number of unique reads, e.g., falling outside of a range of from 2 million to 10 million total sequence reads, from 3 million to 9 million sequence reads, from 3 million to 9 million sequence reads, and the like.
A criterion for unique read depth across the panel, defined as a measure of central tendency (e.g., a mean or median) for a distribution of the number of unique reads in the sequencing reaction encompassing the genomic regions targeted by each probe. Generally, a unique read depth threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a maximum unique read depth threshold of no more than 1500, 1750, 2000, 2250, 2500, 2750, 3000, 3250, 3500, or higher unique read depth. That is, the criterion for failing the sample is satisfied when the unique read depth across the panel for the sequencing reaction is below the maximum total read threshold. In some embodiments, the criterion is implemented as falling outside of a range of acceptable unique read depth, e.g., falling outside of a unique read depth range of from 1000 to 4000, from 1500 to 4000, from 1500 to 4000, and the like.
A criterion for the unique read depth of a lowest percentile across the panel, defined as a measure of central tendency (e.g., a mean or median) for a distribution of the number of unique reads in the sequencing reaction encompassing the genomic regions targeted by each probe that fall within the lowest percentile of genomic regions by read depth (e.g., the first, second, third, fourth, fifth, tenth, fifteenth, twentieth, twenty-fifth, or similar percentile). Generally, a unique read depth at a lowest percentile threshold will be selected based on the sequencing technology used, the size of the targeted panel, the lowest percentile selected, and the expected number of sequence reads generated by the combination of the technology and targeted panel used. For example, in some embodiments where next generation sequencing-by-synthesis technology is used, the criterion is implemented as a maximum unique read depth threshold at the fifth percentile of no more than 500, 750, 1000, 1250, 1500, 1750, 2000, 2250, 2500, or higher unique read depth. That is, the criterion for failing the sample is satisfied when the unique read depth at a lowest percentile threshold for the sequencing reaction is below the maximum unique read depth at a lowest percentile threshold. In some embodiments, the criterion is implemented as falling outside of a range of acceptable unique read depth at the fifth percentile, e.g., falling outside of a unique read depth at the fifth percentile range of from 250 to 3000, from 500 to 3000, from 500 to 2500, and the like.
A criterion for the deamination or OxoG Q-score of a sequencing reaction, defined as a Q-score for the occurrence of artifacts arising from template oxidation/deamination. Generally, a deamination or OxoG Q-score threshold will be selected based on the sequencing technology used. For example, in some embodiments where next generation sequencing-by-synthesis technology is used, the criterion is implemented as a maximum deamination or OxoG Q-score threshold of no more than 10, 20, 30, 40, 5,0 6,0 70, 80, 90, or higher. That is, the criterion for failing the sample is satisfied when the deamination or OxoG Q-score for the sequencing reaction is below the maximum deamination or OxoG Q-score threshold. In some embodiments, the criterion is implemented as falling outside of a range of acceptable deamination or OxoG Q-scores, e.g., falling outside of a deamination or OxoG Q-score range of from 10 to 100, from 10 to 90, and the like.
A criterion for the estimated contamination fraction is of a sequencing reaction, defined as an estimate of the fraction of template fragments in the sample being sequenced arising from contamination of the sample, commonly expressed as a decimal, e.g., where 1% contamination is expressed as 0.01. An example method for estimating contamination in a sequencing method is described in Jun G. et al., Am. J. Hum. Genet., 91:839-48 (2012). For example, in some embodiments, the criterion is implemented as a minimum contamination fraction threshold of at least 0.001, 0.0015, 0.002, 0.0025, 0.003, 0.0035, 0.004. That is, the criterion for failing the sample is satisfied when the contamination fraction for the sequencing reaction is above the minimum contamination fraction threshold. In some embodiments, the criterion is implemented as falling outside of a range of acceptable contamination fractions, e.g., falling outside of a contamination fraction range of from 0.0005 to 0.005, from 0.0005 to 0.004, from 0.001 to 0.004, and the like.
A criterion for the fingerprint correlation score of a sequencing reaction, defined as a Pearson correlation coefficient calculated between the variant allele fractions of a set of pre-defined single nucleotide polymorphisms (SNPs) in two samples. An example method for determining a fingerprint correlation score is described in Sejoon L. et al., Nucleic Acids Research, Volume 45, Issue 11, 20 Jun. 2017, Page e103. For example, in some embodiments, the criterion is implemented as a maximum fingerprint correlation score threshold of no more than 0.1, 0.2, 0.3, 0.4, 0.5, 0.6, 0.7, 0.8, 0.9, or higher. That is, the criterion for failing the sample is satisfied when the fingerprint correlation score for the sequencing reaction is below the maximum fingerprint correlation score threshold. In some embodiments, the criterion is implemented as falling outside of a range of acceptable fingerprint correlation scores, e.g., falling outside of a fingerprint correlation range of from 0.1 to 0.9, from 0.2 to 0.9, from 0.3 to 0.9, and the like.
A criterion for the raw coverage of a minimum percentage of the genomic regions targeted by a probe, defined as a minimum number of unique reads in the sequencing reaction encompassing each of a minimum percentage (e.g., at least 80%, 85%, 90%, 95%, 98%, 99%, 99.5%, 99.9%, and the like) of the genomic regions targeted by the probe panel. Generally, a raw coverage of a minimum percentage of the genomic regions targeted by a probe threshold will be selected based on the sequencing technology used, the size of the targeted panel, the minimum percentage selected, and the expected number of sequence reads generated by the combination of the technology and targeted panel used. For example, in some embodiments where next generation sequencing-by-synthesis technology is used, the criterion is implemented as a raw coverage of 95% of the genomic regions targeted by a probe threshold of no more than 500, 750, 1000, 1250, 1500, 1750, 2000, 2250, 2500, or higher unique read depth. That is, the criterion for failing the sample is satisfied when the raw coverage of a minimum percentage of the genomic regions targeted by a probe for the sequencing reaction is below the maximum raw coverage of a minimum percentage of the genomic regions targeted by a probe threshold. In some embodiments, the criterion is implemented as falling outside of a range of acceptable unique read depth for 95% of the genomic regions targeted by a probe, e.g., requiring that the sequencing reaction generate a unique read depth for 95% of the targeted regions falling outside of a range of from 250 to 3000, from 500 to 3000, from 500 to 2500, and the like.
A criterion for the PCR duplication rate of a sequencing reaction, defined as the percentage of sequence reads that arise from the same template molecule as at least one other sequence read generated by the reaction. Generally, a PCR duplication rate threshold will be selected based on the sequencing technology used, the size of the targeted panel, and the expected number of sequence reads generated by the combination of the technology and targeted panel used. For example, in some embodiments where next generation sequencing-by-synthesis technology is used, the criterion is implemented as a maximum PCR duplication rate threshold of at least 91%, 92%,93%, 94%, 95%, 96%, 97%, 98%, 99%, or higher. That is, the criterion for failing the sample is satisfied when the PCR duplication rate for the sequencing reaction is below the maximum PCR duplication rate threshold. In some embodiments, the criterion is implemented as falling outside of a range of acceptable PCR duplication rates, e.g., of from 90% to 100%, from 90% to 99%, from 91% to 99%, and the like.

Thresholds for the auto-pass and auto-fail criteria may be established with reference to one another, but are not necessarily set at the same level. For instance, in some embodiments, samples with a metric that falls between auto-pass and auto-fail criteria may be routed for manual review by a qualified bioinformatics scientist. Samples that are failed either automatically or by manual review may be routed to medical and laboratory teams for final review and can be released for downstream processing at the discretion of the laboratory medical director or designee.

### Systems and Methods for Estimating Circulating Tumor Fraction and Cancer Monitoring

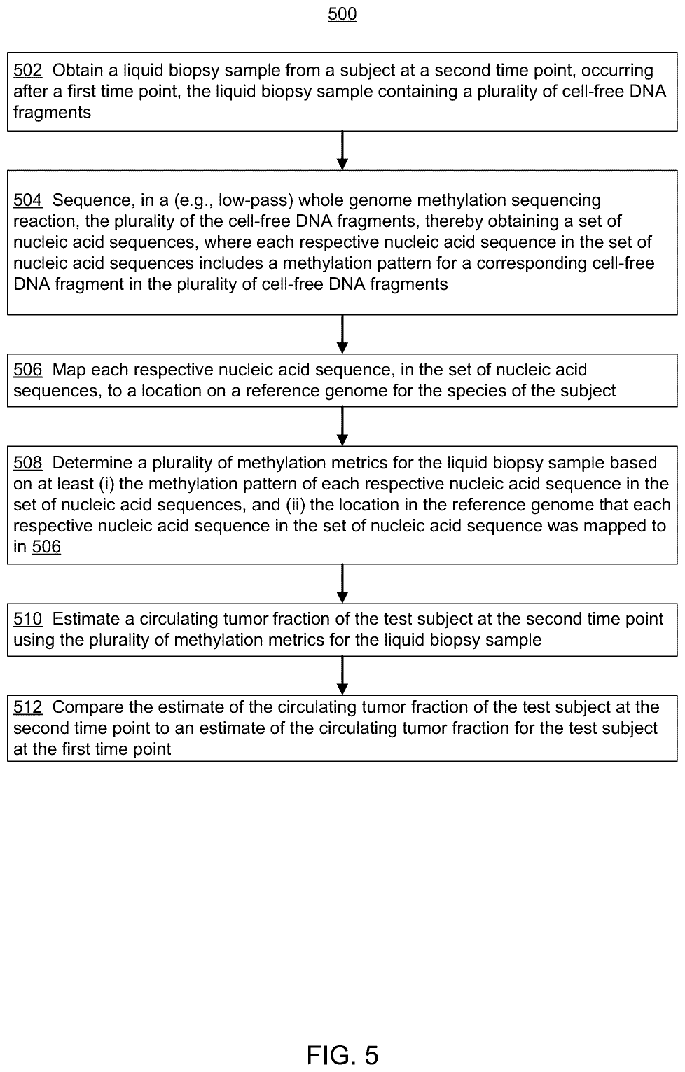

An overview of methods for providing clinical support for personalized cancer therapy is described above with reference to FIGS. 2-4 above. Below, systems and methods for improving estimating circulating tumor fraction and cancer monitoring using low-pass whole genome methylation sequencing, e.g., within the context of the methods and systems described above, are described with reference to FIGS. 5-9.

Many of the embodiments described below, in conjunction with FIGS. 5-9, relate to analyses performed using sequencing data for cfDNA obtained from a liquid biopsy sample of a cancer patient. Generally, these embodiments are independent and, thus, not reliant upon any particular DNA sequencing methods. However, in some embodiments, the methods described below include one or more steps of generating the sequencing data.

Referring to method 500, the present disclosure provides a method for monitoring a cancer condition of a test subject. The method includes obtaining (502) a liquid biopsy sample from a subject at a second time point, occurring after a first time point, the liquid biopsy sample containing a plurality of cell-free DNA fragments. The method then includes sequencing (504), in a whole genome methylation sequencing reaction (e.g., in a low-pass whole genome sequencing reaction at an average unique sequencing depth of less than 3× across the entire genome of the species of the test subject), thereby obtaining a set of nucleic acid sequences, where each respective nucleic acid sequence in the set of nucleic acid sequences includes a methylation pattern for a corresponding cell-free DNA fragment in the plurality of cell-free DNA fragments.

Method 500 then includes mapping (506) each respective nucleic acid sequence, in the set of nucleic acid sequences, to a location on a reference genome for the species of the subject. The method then includes determining (508) a plurality of methylation metrics for the liquid biopsy sample based on at least (i) the methylation pattern of each respective nucleic acid sequence in the set of nucleic acid sequences, and (ii) the location in the reference genome that each respective nucleic acid sequence in the set of nucleic acid sequence was mapped to.

Method 500 then includes estimating (510) a circulating tumor fraction of the test subject at the second time point using the plurality of methylation metrics for the liquid biopsy sample. The estimate of circulating tumor fraction for the test subject at the second time is then compared (512) to an estimate of the circulating tumor fraction for the test subject at the first time point.

In some embodiments, the plurality of methylation metrics is determined by assigning, in a first binning operation, each respective nucleic acid sequence in the set of nucleic acid sequences to a respective bin in a plurality of bins based on the location in the reference genome the respective nucleic acid sequence was mapped to, wherein each respective bin in the plurality of bins represents a unique segment of the reference genome. Then it is determined, for each respective bin in the plurality of bins, a respective methylation metric based on the methylation patterns of the respective nucleic acid sequences assigned to the respective bin, thereby generating the plurality of methylation metrics for the liquid biopsy sample.

In some embodiments, each respective methylation metric in the plurality of methylation metrics is based on a comparison of at least: (i) the quantity of putative methylation sites, in the respective nucleic acid sequences assigned to the corresponding bin in the plurality of bins, that are methylated, and (ii) the quantity of putative methylation sites, in the respective nucleic acid sequences assigned to the corresponding bin in the plurality of bins, that are not methylated.

In some embodiments, the putative methylation sites comprise each CpG dinucleotide represented in the nucleic acid sequences assigned to the corresponding bin in the plurality of bins. In some embodiments, the putative methylation sites comprise each CHG trinucleotide represented in the nucleic acid sequences assigned to the corresponding bin in the plurality of bins, wherein H is an A, T, or C nucleotide. In some embodiments, the putative methylation sites comprise each CHH trinucleotide represented in the nucleic acid sequences assigned to the corresponding bin in the plurality of bins, wherein H is an A, T, or C nucleotide.

In some embodiments, the circulating tumor fraction is estimated by comparing the plurality of methylation metrics against methylation metrics from a plurality of reference subjects with cancer.

In some embodiments, the plurality of methylation metrics is determined by, for each respective nucleic acid sequence in the set of nucleic acid sequences, a respective sequence probability value that the DNA fragment corresponding to the respective nucleic acid sequence was from a cancerous cell using a probabilistic mixture model based on at least the methylation pattern of the respective nucleic acid sequence. In some embodiments, the probabilistic mixture model is also based on a fragmentation pattern of the respective nucleic acid sequence.

In some embodiments, the putative methylation sites comprise each CpG dinucleotide represented in the nucleic acid sequences assigned to the corresponding bin in the plurality of bins. In some embodiments, the putative methylation sites comprise each CHG trinucleotide represented in the nucleic acid sequences assigned to the corresponding bin in the plurality of bins, wherein H is an A, T, or C nucleotide. In some embodiments, the putative methylation sites comprise each CHH trinucleotide represented in the nucleic acid sequences assigned to the corresponding bin in the plurality of bins, wherein H is an A, T, or C nucleotide.

In some embodiments, the circulating tumor fraction is estimated by assigning, in a first binning operation, each respective nucleic acid sequence in the set of nucleic acid sequences to a respective bin in either a first plurality of bins corresponding to germline fragments or a second plurality of bins corresponding to somatic fragments. Each respective bin in the first plurality of bins represents a unique segment of the reference genome. Each respective bin in the second plurality of bins represents the same unique segment of the reference genome as a corresponding bin in the first plurality of bins. The assignment of each respective nucleic acid sequence to a corresponding bin is based on (i) the location in the reference genome the respective nucleic acid sequence was mapped to and (ii) the respective sequence probability value assigned to the respective nucleic acid sequence. Then, for each respective bin in the first plurality of bins, at least the following are compared, (i) a respective first metric representative of the number of nucleic acid sequences assigned to the respective bin in the first plurality of bins and (ii) a respective second metric representative of the number of nucleic acid sequences assigned to the respective bin in the second plurality of bins that corresponds to the respective bin in the first plurality of bins, thereby estimating the circulating tumor fraction of the test subject at the second time point.

In some embodiments, the method includes determining, for each respective bin in the second plurality of bins, a respective copy number representing the average copy number of loci within the segment of the cancer genome of the test subject corresponding to the unique segment of the reference genome represented by the respective bin, and the respective second metric is normalized based on the copy number determined for the respective bin in the second plurality of bins.

In some embodiments, the probabilistic mixture model is trained on methylation data from a plurality of training subjects with cancer. In some embodiments, the test subject had not been diagnosed with cancer prior to the second time point. In other embodiments, the test subject underwent therapy for cancer at the second time point, and the test subject developed the cancer prior to the first time point. In some embodiments, the test subject was believed to be in remission from cancer at the second time point, and the first time point was after the subject had been deemed to be in remission.

In some embodiments, the liquid biopsy sample is a blood sample of the test subject. In some embodiments, the test subject is a human.

In some embodiments, the circulating tumor fraction estimate for the subject at the first time point was based on analysis of cell-free DNA fragments from a liquid biopsy sample obtained from the test subject at the first time point.

In some embodiments, the circulating tumor fraction estimate for the subject at the first time point was based on analysis of low-pass whole genome methylation sequencing of the cell-free DNA fragments from the liquid biopsy sample obtained from the test subject at the first time point.

In some embodiments, the circulating tumor fraction estimate for the subject at the first time point was based on analysis of low-pass whole genome sequencing of the cell-free DNA fragments from the liquid biopsy sample obtained from the test subject at the first time point.

In some embodiments, the circulating tumor fraction estimate for the subject at the first time point was based on analysis of whole exome sequencing of the cell-free DNA fragments from the liquid biopsy sample obtained from the test subject at the first time point.

In some embodiments, the circulating tumor fraction estimate for the subject at the first time point was based on analysis of target-enriched panel sequencing of the cell-free DNA fragments from the liquid biopsy sample obtained from the test subject at the first time point.

In some embodiments, the circulating tumor fraction estimate for the subject at the first time point was based on analysis of DNA from a solid tumor sample obtained from the test subject at the first time point.

In some embodiments, the circulating tumor fraction estimate for the subject at the first time point was based on analysis of whole genome sequencing of DNA from a solid tumor DNA sample obtained from the test subject at the first time point.

In some embodiments, the circulating tumor fraction estimate for the subject at the first time point was based on analysis of whole exome sequencing of DNA from a solid tumor sample obtained from the test subject at the first time point.

In some embodiments, the circulating tumor fraction estimate for the subject at the first time point was based on analysis of whole genome sequencing of DNA from a solid tumor sample obtained from the test subject at the first time point.

In some embodiments, the estimate of the circulating tumor fraction for the subject at the first time point was based on analysis of matched liquid biopsy and solid tumor samples obtained from the test subject at the first time point.

In some embodiments, the method also includes assigning, in a second binning operation, each respective nucleic acid sequence in the set of nucleic acid sequences to a respective bin in a third plurality of bins. Each respective bin in the third plurality of bins represents a unique segment of the reference genome. The assignment of each respective nucleic acid sequence to a corresponding bin is based on the location in the reference genome the respective nucleic acid sequence was mapped to. It is determined, for each respective bin in the third plurality of bins, a bin-level size-distribution metric based on a characteristic of the distribution of the fragment lengths of cell-free DNA fragments corresponding to the nucleic acid sequences assigned to the respective bin, thereby obtaining a set of bin-level size-distribution metrics. The circulating tumor fraction of the test subject is then estimated based on the set of bin-level size distribution metrics, thereby generating a fragment length-based estimate of the circulating tumor fraction of the test subject at the second time point.

In some embodiments, the method includes assigning, in a fourth binning operation, each respective nucleic acid sequence in the set of nucleic acid sequences to a respective bin in a fourth plurality of bins. Each respective bin in the fourth plurality of bins represents a unique segment of the reference genome. The assignment of each respective nucleic acid sequence to a corresponding bin is based on the location in the reference genome the respective nucleic acid sequence was mapped to. It is then determined, for each respective bin in the fourth plurality of bins, a fragment copy number associated with the number of nucleic acid sequences assigned to the respective bin, thereby obtaining a set of bin-level fragment copy number metrics. The set of bin-level fragment copy metrics are modeled using a statistical model of copy number alterations to estimate the circulating tumor fraction of the test subject, thereby generating a copy number-based estimate of the circulating tumor fraction of the test subject.

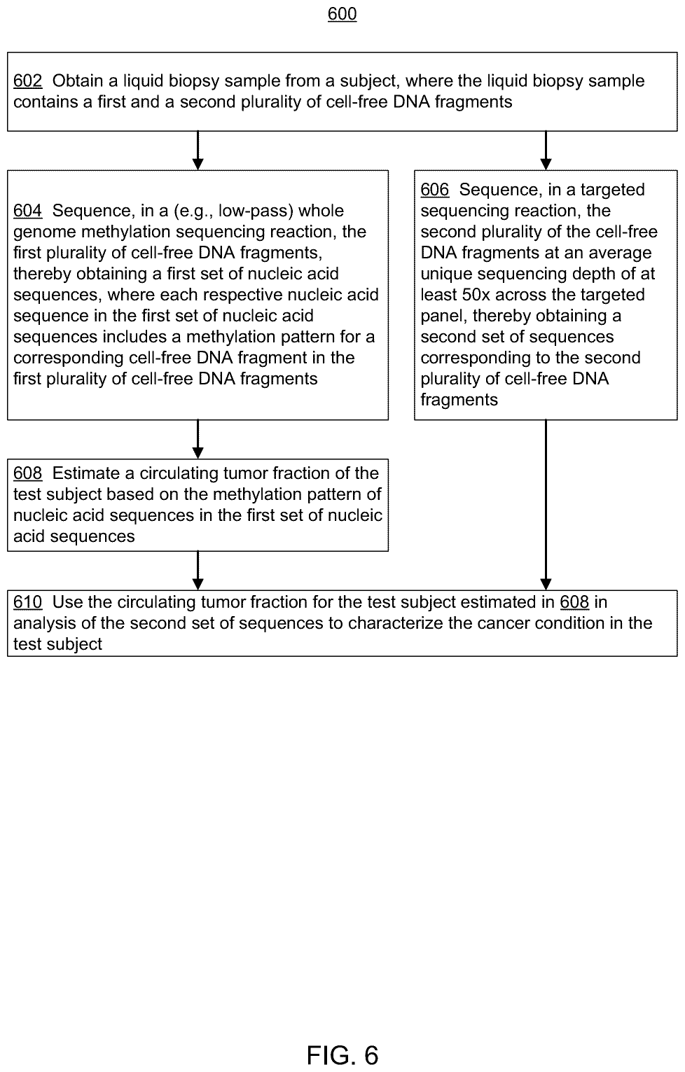

Referring to method 600 illustrated in FIG. 6, in some embodiments, the present disclosure provides a method of characterizing a cancer condition of a test subject from a liquid biopsy sample. Specifically, method 600 uses a circulating tumor fraction estimate from whole genome methylation sequencing data of cfDNA in the liquid biopsy sample, e.g., using an ensemble model 1100 for circulating tumor fraction as described herein, to inform evaluation of sequencing data from a non-methylation sequencing reaction of cfDNA in the liquid biopsy sample.

Accordingly, method 600 optionally includes obtaining (602) a liquid biopsy sample from a test subject, where the liquid biopsy sample contains a first and a second plurality of cell-free DNA fragments. In some embodiments, the liquid biopsy sample is a blood, whole blood, plasma, serum, urine, cerebrospinal fluid, fecal, saliva, sweat, tears, pleural fluid, pericardial fluid, or peritoneal fluid sample from the test subject. In some embodiments, the liquid biopsy sample is a blood sample of the test subject. In some embodiments, the test subject is a human.

Method 600 then includes sequencing (604), in a (e.g., low-pass) whole genome methylation sequencing reaction, the first plurality of cell-free DNA fragments, thereby obtaining a first set of nucleic acid sequences. Each respective nucleic acid sequence in the first set of nucleic acid sequences includes a methylation pattern for a corresponding cell-free DNA fragment in the first plurality of cell-free DNA fragments. In some embodiments, the whole genome methylation sequencing reaction is performed at an average unique sequencing depth of less than 3× across the entire genome of the species of the test subject. In other embodiments, the whole genome methylation sequencing reaction is performed at an average unique sequencing depth of less than 25×, less than 20×, less than 15×, less than 10×, less than 5×, less than 4×, less than 3×, less than 2. less than 5×, less than 2×, less than 1. less than 5×, less than 1×, less than 0.9×, less than 0.8×, less than 0.75×, less than 0.7×, less than 0.5×, or less, across the genome of the species of the test subject. In some embodiments, the whole genome methylation sequencing reaction is performed at an average unique sequencing depth of at least 1×, at least 1.5×, at least 2×, at least 2.5×, at least 3×, at least 4×, at least 5×, at least 10×, at least 15×, at least 20×, at least 25×, at least 30×, at least 35×, at least 40×, at least 45×, at least 50×, at least 75×, at least 100×, or greater, across the genome of the species of the test subject.

Method 600 also includes sequencing (606), e.g., in a targeted sequencing reaction, the second plurality of the cell-free DNA fragments thereby obtaining a second set of sequences corresponding to the second plurality of cell-free DNA fragments. In some embodiments, the second sequencing reaction is performed at an average unique sequencing depth of at least 50× across the targeted panel. In some embodiments, the second sequencing reaction is performed at an average unique sequencing depth of at least 10×, at least 15×, at least 20×, at least 25×, at least 30×, at least 35×, at least 40×, at least 50×, at least 75×, at least 100×, or more across the targeted panel.

In some embodiments, the targeted panel includes probes against at least 10 genes, at least 25 genes, at least 50 genes, at least 75 genes, at least 100 genes, at least 125 genes, at least 150 genes, at least 175 genes, at least 200 genes, at least 250 genes, at least 300 genes, at least 400 genes, at least 500 genes or more genes. In some embodiments, the targeted panel includes probes against no more than 1500 genes, no more than 1250 genes, no more than 1000 genes, no more than 900 genes, no more than 800 genes, no more than 750 genes, no more than 700 genes, no more than 600 genes, no more than 500 genes, no more than 400 genes, no more than 300 genes, no more than 250 genes, no more than 200 genes, no more than 175 genes, no more than 150 genes, no more than 125 genes, no more than 100 genes, or fewer.

Method 600 also includes estimating (608) the circulating tumor fraction of the test subject, based on at least the methylation pattern of nucleic acid sequences in the first set of nucleic acid sequences. In some embodiments, the circulating tumor fraction is estimated using an ensemble model, such as model 1100 described herein.

In some embodiments, estimating the circulating tumor fraction includes mapping each respective nucleic acid sequence, in the first set of nucleic acid sequences, to a location on a reference genome for the species of the subject, determining a plurality of methylation metrics for the liquid biopsy sample based on at least (i) the methylation pattern of each respective nucleic acid sequence in the set of nucleic acid sequences, and (ii) the location in the reference genome that each respective nucleic acid sequence in the set of nucleic acid sequence maps to, and estimating the circulating tumor fraction of the test subject using the plurality of methylation metrics for the liquid biopsy sample.

In some embodiments, the plurality of methylation metrics are determined by assigning, in a first binning operation, each respective nucleic acid sequence in the set of nucleic acid sequences to a respective bin in a plurality of bins based on the location in the reference genome the respective nucleic acid sequence maps to, where each respective bin in the plurality of bins represents a unique segment of the reference genome. Then, in some embodiments, a respective methylation metric is determined, for each respective bin in the plurality of bins, based on the methylation patterns of the respective nucleic acid sequences assigned to the respective bin, thereby generating the plurality of methylation metrics for the liquid biopsy sample. In some embodiments, the plurality of bins includes at least 50 bins, at least 100, at least 150, at least 200, at least 250, at least 300, at least 400, at least 500, at least 750, at least 1000, at least 5000, at least 10,000, at least 25,000, at least 50,000, at least 75,000, at least 100,000, or more bins. In some embodiments, the plurality of bins collectively cover at least 1 Mb, at least 2 Mb, at least 3 Mb, at least 4 Mb, at least 5 Mb, at least 10 Mb, at least 15 Mb, at least 20 Mb, at least 25 Mb, at least 30 Mb, at least 40 Mb, at least 50 Mb, at least 75 Mb, at least 100 Mb, at least 250 Mb, at least 500 Mb, or more of the genome of the species of the subject.

In some embodiments, a respective methylation metric in the plurality of methylation metrics is based on a comparison of at least: (i) the quantity of putative methylation sites, in the respective nucleic acid sequences assigned to the corresponding bin in the plurality of bins, that are methylated, and (ii) the quantity of putative methylation sites, in the respective nucleic acid sequences assigned to the corresponding bin in the plurality of bins, that are not methylated.

In some embodiments, the putative methylation sites comprise each CpG dinucleotide represented in the nucleic acid sequences assigned to the corresponding bin in the plurality of bins. In some embodiments, the putative methylation sites comprise each CHG trinucleotide represented in the nucleic acid sequences assigned to the corresponding bin in the plurality of bins, wherein H is an A, T, or C nucleotide. In some embodiments, the putative methylation sites comprise each CHH trinucleotide represented in the nucleic acid sequences assigned to the corresponding bin in the plurality of bins, wherein H is an A, T, or C nucleotide.

In some embodiments, the circulating tumor fraction is estimated by comparing the plurality of methylation metrics against a methylation metrics from a plurality of reference subjects with cancer.

In some embodiments, the plurality of methylation metrics is determined by, for each respective nucleic acid sequence in the set of nucleic acid sequences, a respective sequence probability value that the DNA fragment corresponding to the respective nucleic acid sequence was from a cancerous cell. In some embodiments, the probability value is determined using a probabilistic model based on at least the methylation pattern of the respective nucleic acid sequence. For example, Li W., et al., describe a model in which the joint methylation states of multiple adjacent CpG sites on an individual sequence read are modeled probabilistically based on Beta distributions established for the methylation patterns of the genomic sequence represented in the sequence read from (i) methylation sequencing reactions of a cohort of training subject with cancer, e.g., having the same type of cancer as the test subject, and (ii) methylation sequencing reactions of a cohort of training subjects without cancer. See, Li W., et al., Nucleic Acids Research, 46(15):e89 (2018), which is incorporated herein by reference in its entirety for all purposes, and specifically for its disclosure of a probabilistic model for determining a probability that a particular genomic methylation pattern is derived from a cancerous tissue or a non-cancerous tissue in a test subject. In some embodiments, the probabilistic model is also based on a fragmentation pattern of the respective nucleic acid sequence.

In some embodiments, the putative methylation sites comprise each CpG dinucleotide represented in the nucleic acid sequences assigned to the corresponding bin in the plurality of bins. In some embodiments, the putative methylation sites comprise each CHG trinucleotide represented in the nucleic acid sequences assigned to the corresponding bin in the plurality of bins, wherein H is an A, T, or C nucleotide. In some embodiments, the putative methylation sites comprise each CHH trinucleotide represented in the nucleic acid sequences assigned to the corresponding bin in the plurality of bins, wherein H is an A, T, or C nucleotide.

In some embodiments, the circulating tumor fraction is estimated by assigning, in a first binning operation, each respective nucleic acid sequence in the set of nucleic acid sequences to a respective bin in either a first plurality of bins corresponding to germline fragments or a second plurality of bins corresponding to somatic fragments. Each respective bin in the first plurality of bins represents a unique segment of the reference genome. Each respective bin in the second plurality of bins represents the same unique segment of the reference genome as a corresponding bin in the first plurality of bins. The assignment of each respective nucleic acid sequence to a corresponding bin is based on (i) the location in the reference genome the respective nucleic acid sequence was mapped to and (ii) the respective sequence probability value assigned to the respective nucleic acid sequence. Then, for each respective bin in the first plurality of bins, at least the following are compared, (i) a respective first metric representative of the number of nucleic acid sequences assigned to the respective bin in the first plurality of bins and (ii) a respective second metric representative of the number of nucleic acid sequences assigned to the respective bin in the second plurality of bins that corresponds to the respective bin in the first plurality of bins, thereby estimating the circulating tumor fraction of the test subject at the second time point.

In some embodiments, the method includes determining, for each respective bin in the second plurality of bins, a respective copy number representing the average copy number of loci within the segment of the cancer genome of the test subject corresponding to the unique segment of the reference genome represented by the respective bin, and the respective second metric is normalized based on the copy number determined for the respective bin in the second plurality of bins.

Method 600 then includes using (610) the circulating tumor fraction for the test subject estimated using the first set of nucleic acid sequences in analysis of the second set of sequences to characterize the cancer condition in the test subject.

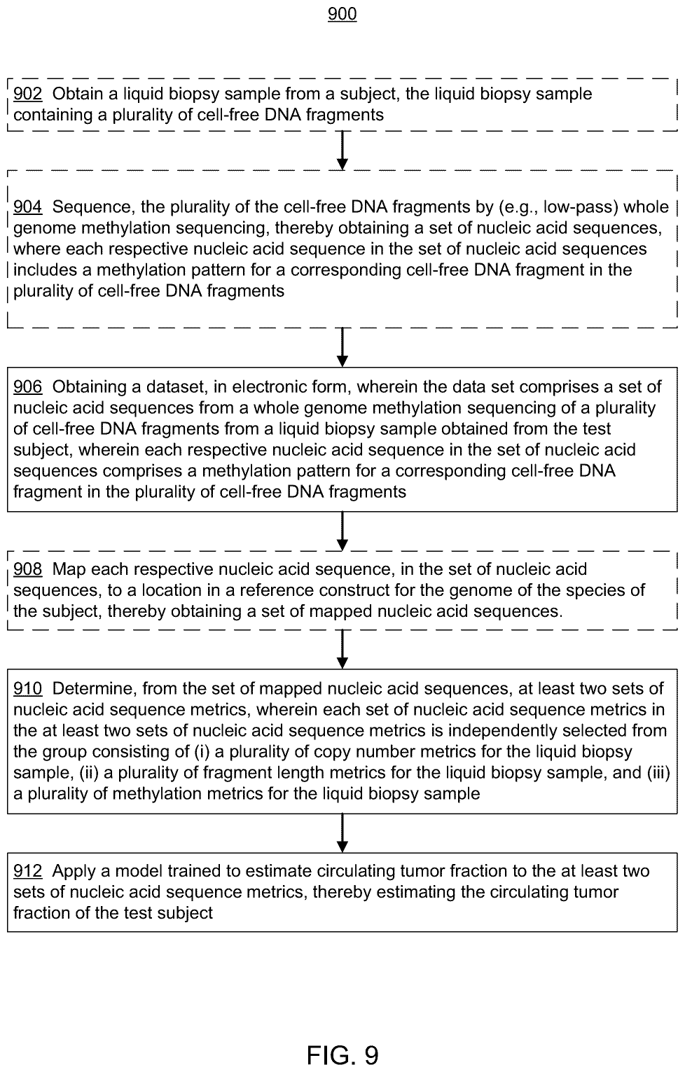

Referring to method 900 illustrated in FIG. 9, the present disclosure provides a method for estimating a circulating tumor fraction of a test subject using an ensemble classifier.

In some embodiments, method 900 includes an optional step of obtaining (902) a liquid biopsy sample from a subject, the liquid biopsy sample containing a plurality of cell-free DNA fragments. Similarly, in some embodiments, method 900 includes a step of sequencing (904) the plurality of the cell-free DNA fragments by whole genome methylation sequencing (e.g., in a low-pass whole genome methylation sequencing reaction at an average unique sequencing depth of less than 3× across the entire genome of the species of the test subject), thereby obtaining a set of nucleic acid sequences, where each respective nucleic acid sequence in the set of nucleic acid sequences includes a methylation pattern for a corresponding cell-free DNA fragment in the plurality of cell-free DNA fragments.

In some embodiments, the whole genome methylation sequencing reaction is performed at an average unique sequencing depth of less than 25×, less than 20×, less than 15×, less than 10×, less than 5×, less than 4×, less than 3×, less than 2. less than 5×, less than 2×, less than 1. less than 5×, less than 1×, less than 0.9×, less than 0.8×, less than 0.75×, less than 0.7×, less than 0.5×, or less, across the genome of the species of the test subject. In some embodiments, the whole genome methylation sequencing reaction is performed at an average unique sequencing depth of at least 1×, at least 1.5×, at least 2×, at least 2.5×, at least 3×, at least 4×, at least 5×, at least 10×, at least 15×, at least 20×, at least 25×, or greater, across the genome of the species of the test subject. In some embodiments, the whole genome sequencing is performed at an average unique sequencing depth of from 0.25× to 3×, from 0.5× to 3×, from 1× to 3×, from 0.25× to 2×, from 0.5× to 2×, from 1× to 2×, from 0.25× to 1.5×, from 0.5× to 1.5×, from 1× to 1.5×, from 0.25× to 1×, or from 0.5× to 1× across the entire genome of the species of the test subject.

However, in some embodiments, method 900 begins with obtaining, e.g., in electronic form, raw sequence reads and/or de-duplicated nucleic acid sequences previously obtained from a whole genome methylation sequencing reaction, rather than obtaining the sample and/or performing the sequencing reaction. Accordingly, method 900 includes obtaining (906) a dataset, in electronic form, where the dataset includes a set of nucleic acid sequences from a whole genome methylation sequencing of a plurality of cell-free DNA fragments from a liquid biopsy sample obtained from the test subject, where each respective nucleic acid sequence in the set of nucleic acid sequences includes a methylation pattern for a corresponding cell-free DNA fragment in the plurality of cell-free DNA fragments. In some embodiments, the set of nucleic acid sequences includes at least 10,000 nucleic acid sequences. In some embodiments, the set of nucleic acid sequences includes at least 1000, at least 2500, at least 5000, at least 7500, at least 10,000, at least 25,000, at least 50,000, at least 100,000, at least 250,000, at least 500,000, at least 1,000,000, at least 2,500,000, at least 5,000,000, at least 10,000,000, or more nucleic acid sequences.

In some embodiments, the set of nucleic acid sequences obtained for the test subject has already been mapped to a location on a reference construct for the species of the test subject. However, in some embodiments, the set of nucleic acid sequences obtained for the test subject has not already been mapped to a location on a reference construct for the species of the test subject. Accordingly, in some embodiments, method 900 includes a step of mapping (908) each respective nucleic acid sequence, in the set of nucleic acid sequences, to a location in a reference construct for the genome of the species of the subject, thereby obtaining a set of mapped nucleic acid sequences.

Method 900 then includes determining (910), from the set of mapped nucleic acid sequences, at least two sets of nucleic acid sequence metrics, where each set of nucleic acid sequence metrics in the at least two sets of nucleic acid sequence metrics is independently selected from the group consisting of (i) a plurality of copy number metrics for the liquid biopsy sample, (ii) a plurality of fragment length metrics for the liquid biopsy sample, and (iii) a plurality of methylation metrics for the liquid biopsy sample.

Method 900 then includes applying (912) a model trained to estimate circulating tumor fraction to the at least two sets of nucleic acid sequence metrics, thereby estimating the circulating tumor fraction of the test subject. In some embodiments, the model is an ensemble model that includes one or more respective component models for each respective set of nucleic acid sequence metrics in the at least two sets of nucleic acid sequence metrics. In some embodiments, the ensemble model generates a corresponding component circulating tumor fraction estimate from each respective component model, and combines the corresponding component circulating tumor fraction estimate from each respect component model to estimate the circulating tumor fraction of the test subject.

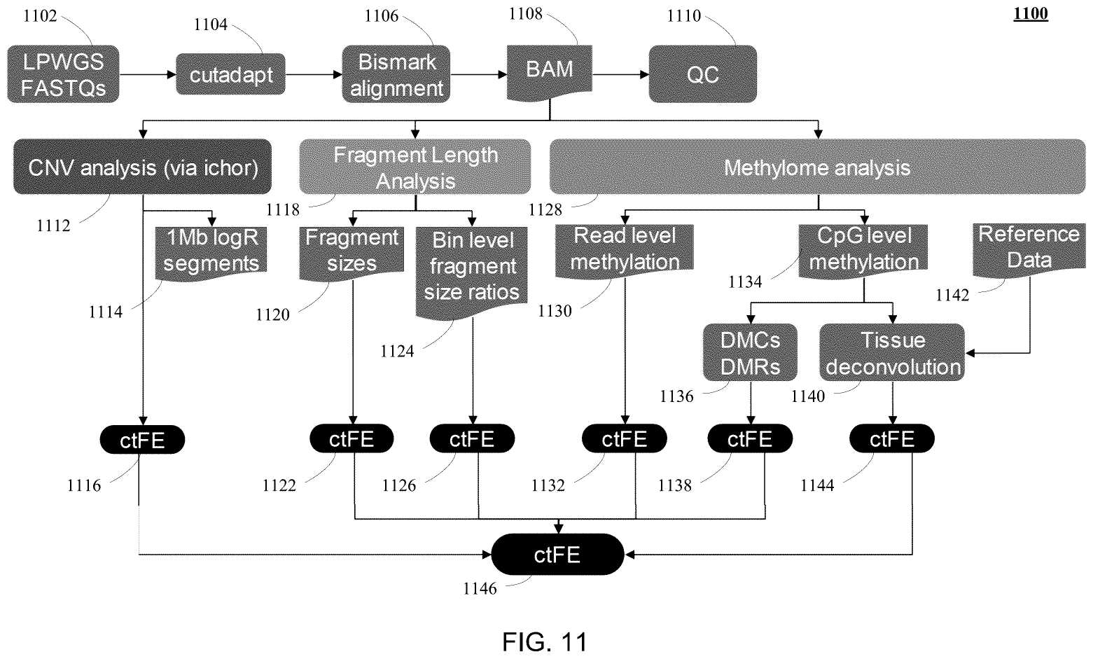

For instance, FIG. 11 illustrates an example ensemble model 1100, that applies two or more of optional component models 1114, 1120, 1124, 1130, 1136, and 1140, e.g., as described in more detail below, to copy number metrics (e.g., component model 1114), fragment length metrics (e.g., component models 1120 and 1124), or methylation metrics (e.g., component models 1130, 1136, and 1140) to generate a component estimate of circulating tumor fraction (e.g., component estimates 1116, 1122, 1126, 1132, 1138, and 1144, respectively). In some embodiments, the ensemble model includes at least two component models selected from component models 1114, 1120, 1124, 1130, 1136, and 1140, as illustrated in FIG. 11 and described in detail below. In some embodiments, the ensemble model includes at least three component models selected from component models 1114, 1120, 1124, 1130, 1136, and 1140, as illustrated in FIG. 11 and described in detail below. In some embodiments, the ensemble model includes at least four component models selected from component models 1114, 1120, 1124, 1130, 1136, and 1140, as illustrated in FIG. 11 and described in detail below. In some embodiments, the ensemble model includes at least five component models selected from component models 1114, 1120, 1124, 1130, 1136, and 1140, as illustrated in FIG. 11 and described in detail below. In some embodiments, the ensemble model includes all six of component models 1114, 1120, 1124, 1130, 1136, and 1140, as illustrated in FIG. 11 and described in detail below. Non-limiting examples of the types of component models, and they ways in which the component models can be combined in the ensemble model, that can be used in the methods and systems provided herein are described herein, for example, in the sections titled “Multi-feature Classifiers and Machine Learning,” and “Ensemble Models.”

In some embodiments, one or more of component models 1114, 1120, 1124, 1130, 1136, and 1140 provides an indication of whether the subject has cancer (e.g., a likelihood, probability, binary indication, etc.), in addition to or instead of a component circulating tumor fraction. In some embodiments, a component model described herein is capable of identifying a cancer signature in the set of nucleic acid sequences at a limit of detection (LOD) when the circulating tumor fraction of the subject is at least 0.001 (i.e., when at least 0.1% of the nucleic acid fragments in the liquid biopsy sample from the test subject are derived from a cancer cell). In some embodiments, a component model described herein is capable of identifying a cancer signature in the set of nucleic acid sequences at a limit of detection (LOD) when the circulating tumor fraction of the subject is at least 0.0001, at least 0.00025, at least 0.0005, at least 0.00075, at least 0.001, at least 0.0015, at least 0.002, at least 0.0025, at least 0.003, at least 0.004, at least 0.005, at least 0.006, at least 0.007, at least 0.007, at least 0.008, at least 0.009, at least 0.01, or greater.

FIG. 11 illustrates an example ensemble model for estimating circulating tumor fraction in a liquid biopsy sample, according to various embodiments of the disclosure. The ensemble model illustrated in FIG. 11 combines component circulating tumor fraction estimates generated from each of component models 1114, 1120, 1124, 1130, 1136, and 1140. However, this is merely illustrative, as any sub-combination of these component models can be used in an ensemble model, as described herein. Further, the skilled artisan will know of other component models that may be used in combination with any of the component models described herein, to form an ensemble model.

In some embodiments, different combinations of component models are used based on characteristics of the subject, the subject's cancer, or a general range of circulating tumor fraction for the sample being sequenced. For example, some component models may perform poorly at very high and/or vary low circulating tumor fractions, or for a particular type of cancer.

Further, the component models of an ensemble model may generally be combined in any fashion know for combining components of an ensemble model. In some embodiments, component models are combined in different fashions based on characteristics of the subject, the subject's cancer, or a general range of circulating tumor fraction for the sample being sequenced.

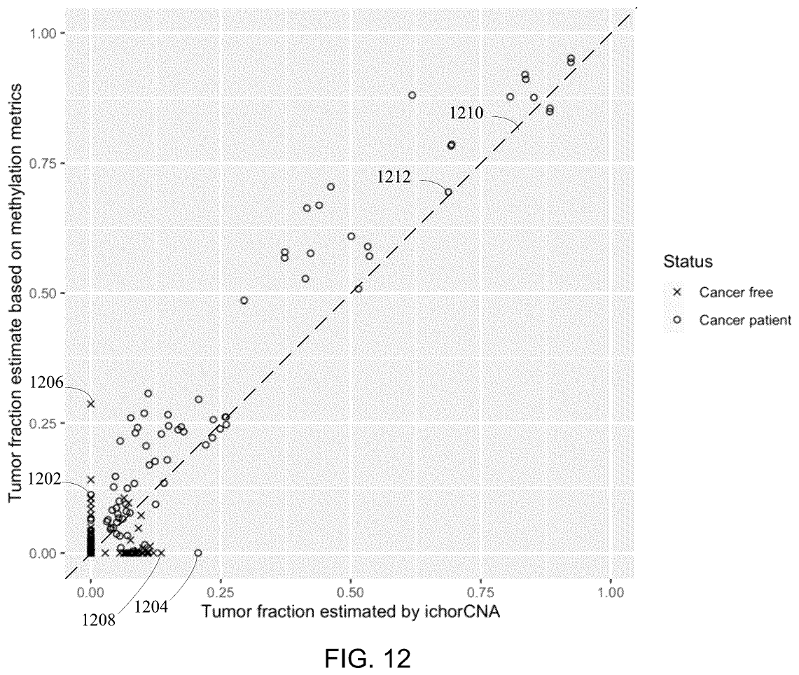

In some embodiments, one or more of the component models described herein is trained as a classifier for identifying a cancer state of a subject, e.g., whether the subject has or does not have cancer. In some embodiments, a classification generated by a classification model further informs an estimation of circulating tumor fraction for a subject. For example, in some embodiments, a classification by one or more classification models that a subject has cancer will inform whether to use a particular component model for estimation of circulating tumor fraction. For instance, as illustrated in FIG. 12, a circulating tumor fraction estimate generated by a particular component model may indicate that there is no circulating tumor fraction, e.g., when the sample has a low circulating tumor fraction. For instance, points 1206 and 1208 in FIG. 12. In such cases, a classification that a subject has cancer, provided by a classification model, may inform exclusion of that component model in an ensemble estimation of the circulating tumor fraction of the subject.

### Component Model 1136—Bin-Level Methylation

In some embodiments, the ensemble model includes a component model trained to generate a component circulating tumor fraction estimate based on a plurality of methylation metrics, e.g., corresponding to component model 1136 as illustrated in FIG. 11. In some embodiments, the plurality of methylation metrics include a plurality of bin-level methylation metrics. Each respective bin-level methylation metric in the plurality of bin-level methylation metrics represents a corresponding genomic region in a first plurality of genomic regions that is differentially methylated in a cancerous tissue relative to a non-cancerous tissue, e.g., differentially methylated CpG dinucleotides and/or differentially methylated genomic regions, for, example as identified using one or more feature selection methodologies described above in the section titled “Multi-feature Classifiers and Machine Learning.” The respective bin-level methylation metric is determined based on a methylation pattern of each respective nucleic acid sequence in the set of mapped nucleic acid sequences that map to the corresponding genomic region.

Non-limiting examples of the types of component models, and they ways in which the component models can be combined in the ensemble model, that can be used in the methods and systems provided herein are described herein, for example, in the sections titled “Multi-feature Classifiers and Machine Learning,” and “Ensemble Models.” In some embodiments, the first component model is a probabilistic model, a deep learning model, or an admixture model.

In some embodiments, a bin-level methylation feature includes a metric for a methylation pattern at one or more putative methylation sites in the sequence reads assigned to a respective bin. In some embodiments, the metric is determined based on an aggregate vote for all instances of the one or more putative methylation sites in the sequence reads assigned to a respective bin. In some embodiments, the metric is determined based on individual votes for the one or more putative methylation sites from each respective sequence read assigned to a respective bin.

For instance, one example of a bin-level methylation feature is a proportion of all putative methylation sites, present in the sequence reads assigned to a respective bin, that are methylated. Another example of a bin-level feature is a proportion of a subset of putative methylation sites (e.g., a subset of one or more putative methylation sites that are differentially methylated in one or more types of cancerous tissue relative to a non-cancerous tissue or one or more different types of cancerous tissue, e.g., a subset of at least 1, 2, 3, 4, 5, 6, 7, 8, 9, 10, 15, 20, 25, 30, 35, 40, 45, 50, 60, 75, 100, 150, 200, 250, 300, 400, 500, 750, 1000, 2500, 500, 10,000, or more putative methylation sites that are differentially methylated in one or more types of cancerous tissue relative to a non-cancerous tissue or one or more different types of cancerous tissue), present in the sequence reads assigned to a respective bin, that are methylated. Another example of a bin-level feature is a measure of central tendency for a metric of the methylation patterns of respective nucleic acid sequences assigned to a respective bin (e.g., an average proportion of putative methylation sites, e.g., of all putative methylation sites or of a subset of putative methylation sites such as those that are differentially methylated in one or more types of cancerous tissue relative to a noncancerous tissue or one or more different types of cancerous tissue, that are methylated in respective nucleic acid sequences). Another example of a bin-level feature is a proportion of sequence reads assigned to a respective bin that have a particular methylation pattern, e.g., that have at least a threshold amount of methylation (e.g., where at least 10%, 15%, 20%, 25%, 30%, 35%, 40%, 45%, 50%, 55%, 60%, 65%, 70%, 75%, 80%, 85%, 90%, 95%, etc., of the putative methylation sites are methylated). Another example of a bin-level feature is a distribution of corresponding probabilities that respective nucleic acid sequences assigned to a respective bin are derived from a cancerous tissue. Another example of a bin-level feature is a summary statistic for the distribution of corresponding probabilities that respective nucleic acid sequences assigned to a respective bin are derived from a cancerous tissue, e.g., a measure of central tendency or a measure of dispersion of the distribution.

In some embodiments, a respective genomic region in the first plurality of genomic regions (e.g., each respective region or a portion of the first plurality of genomic regions) includes a corresponding plurality of putative methylation sites. In some embodiments, each respective genomic region includes at least 2, 3, 4, 5, 6, 7, 8, 9, 10, 15, 20, 25, or more putative methylation sites. Accordingly, in some embodiments, the corresponding bin-level methylation metric for the respective genomic region in the first plurality of genomic regions is based on a comparison of at least: (i) the quantity of the corresponding putative methylation sites in the respective nucleic acid sequences that map to the respective genomic region that are methylated, and (ii) the quantity of the corresponding putative methylation sites in the respective nucleic acid sequences that map to the respective genomic region that are unmethylated.

In some embodiments, a respective genomic region in the first plurality of genomic regions (e.g., each respective region or a portion of the first plurality of genomic regions) includes a single putative methylation site. Accordingly, in some embodiments, the corresponding bin-level methylation metric for the respective genomic region in the first plurality of genomic regions is based on a comparison of at least: (i) the quantity of the corresponding putative methylation sites in the respective nucleic acid sequences that map to the respective genomic region that are methylated, and (ii) the quantity of the corresponding putative methylation sites in the respective nucleic acid sequences that map to the respective genomic region that are unmethylated.

In some embodiments, the plurality of methylation metrics are corrected for DNA methylation degradation prior to the whole genome methylation sequencing or incomplete identification of methylated residues during the whole genome methylation sequencing of the plurality of cell-free DNA fragments. In some embodiments, the correction includes (a) determining, for each respective genomic region in a second plurality of genomic regions, wherein the methylation patterns of each respective genomic region in the second plurality of genomic regions is invariant in cancerous and non-cancerous tissues, a quantity of putative methylation sites, in the respective nucleic acid sequences that map to the corresponding genomic region in the second plurality of genomic regions, that are methylated, (b) determining a divergence between (i) an expected quantity of putative methylation sites, in the respective nucleic acid sequences that map to the corresponding genomic region in the second plurality of genomic regions, that are methylated, and (ii) the determined quantity of putative methylation sites that are methylated, and (c) correcting the plurality of methylation metrics based on the determined divergence.

In some embodiments, the component model accounts for methylation degradation prior to the whole genome methylation sequencing or incomplete identification of methylated residues. In such embodiments, the component model evaluates a first plurality of methylation metrics (for differentially methylated CpG dinucleotides and/or differentially methylated genomic regions) and a second plurality of methylation metrics (for invariantly methylated CpG dinucleotides and/or invariantly methylated genomic regions). An example of such a model, trained using a Markov Chain Monte Carlo (MCMC) methodology is described above in the section titled “Multi-feature Classifiers and Machine Learning.”

In some embodiments, the second plurality of genomic regions is at least 25, at least 50, at least 100, at least 250, at least 500, at least 1000, at least 2500, at least 5000, at least 10,000, at least 25,000, at least 50,000, at least 100,000, at least 250,000, or more genomic regions.

In some embodiments, for a respective genomic region in the first plurality of genomic regions, the corresponding plurality of putative methylation sites comprise each CpG dinucleotide represented in the nucleic acid sequences that map to the respective genomic region. In some embodiments, the corresponding plurality of putative methylation sites comprise each CHG trinucleotide represented in the nucleic acid sequences that map to the respective genomic region, wherein H is an A, T, or C nucleotide. In some embodiments, the corresponding plurality of putative methylation sites comprise each CHH trinucleotide represented in the nucleic acid that map to the respective genomic region, wherein H is an A, T, or C nucleotide.

In some embodiments, the plurality of genomic regions is at least 100, at least 250, at least 500, at least 1000, at least 2500, at least 5000, at least 10,000, at least 25,000, at least 50,000, at least 100,000, at least 250,000, or more genomic regions that are differentially methylated in a cancerous tissue relative to a non-cancerous tissue.

In some embodiments, the model is specific for a single type of cancer. For instance, in some embodiments, the test subject has been diagnosed with a respective cancer type in a plurality of cancer types, and the first plurality of genomic regions are differentially methylated in the respective cancer type relative to a non-cancerous tissue.

### Component Model 1130—Fragment-Level Methylation

In some embodiments, the ensemble model includes a component model trained to generate a component circulating tumor fraction estimate based on a plurality of methylation metrics, e.g., corresponding to component model 1130 as illustrated in FIG. 11. In some embodiments, the plurality of methylation metrics comprises a plurality of fragment-level methylation metrics. That is, one or more characteristics of the methylation pattern of individual DNA fragments in the liquid biopsy sample. Accordingly, in some embodiments, each respective fragment-level methylation metric in the plurality of fragment-level methylation metrics represents a respective nucleic acid sequence, in at least a subset of the set of mapped nucleic acid sequences, that map to a respective genomic region in a third plurality of genomic regions that is differentially methylated in a cancerous tissue relative to a non-cancerous tissue.

Non-limiting examples of the types of component models, and they ways in which the component models can be combined in the ensemble model, that can be used in the methods and systems provided herein are described herein, for example, in the sections titled “Multi-feature Classifiers and Machine Learning,” and “Ensemble Models.” In some embodiments, the first component model is a probabilistic model, a deep learning model, or an admixture model.

In some embodiments, the respective fragment-level methylation metric includes a respective probability value that the DNA fragment corresponding to the respective nucleic acid sequence was from a cancerous cell based on at least the methylation pattern of the respective nucleic acid sequence. In some embodiments, the probability value is determined using a probabilistic model based on at least the methylation pattern of the respective nucleic acid sequence. For example, Li W., et al., describe a model in which the joint methylation states of multiple adjacent CpG sites on an individual sequence read are modeled probabilistically based on Beta distributions established for the methylation patterns of the genomic sequence represented in the sequence read from (i) methylation sequencing reactions of a cohort of training subject with cancer, e.g., having the same type of cancer as the test subject, and (ii) methylation sequencing reactions of a cohort of training subjects without cancer. See, Li W., et al., Nucleic Acids Research, 46(15):e89 (2018), which is incorporated herein by reference in its entirety for all purposes, and specifically for its disclosure of a probabilistic model for determining a probability that a particular genomic methylation pattern is derived from a cancerous tissue or a non-cancerous tissue in a test subject. In some embodiments, the respective probability value is assigned based on (i) the methylation pattern of the respective nucleic acid sequence, and (ii) the length of the DNA fragment corresponding to the respective nucleic acid sequence.

In some embodiments, the respective probability value is assigned based on fitting the methylation pattern of, and optionally the length of the DNA fragment corresponding to, the respective nucleic acid sequence to one of a first DNA fragment distribution for DNA fragments originating from cancerous cells and a second DNA fragment distribution for DNA fragments originating from non-cancerous cells using a probabilistic model, deep learning model, or admixture model.

In some embodiments, the third plurality of genomic regions is at least 25, at least 50, at least 100, at least 250, at least 500, at least 1000, at least 2500, at least 5000, at least 10,000, at least 25,000, at least 50,000, at least 100,000, at least 250,000, or more genomic regions.

In some embodiments, the model is specific for a single type of cancer. For instance, in some embodiments, the test subject has been diagnosed with a respective cancer type in a plurality of cancer types, and the first plurality of genomic regions are differentially methylated in the respective cancer type relative to a non-cancerous tissue.

In some embodiments, component model 1130 provides an indication of whether the subject has cancer (e.g., a likelihood, probability, binary indication, etc.), in addition to or instead of a component circulating tumor fraction. In some embodiments, component model 1130 determines how many of the respective nucleic acid sequences, in at least a subset of the set of mapped nucleic acid sequences, that map to a respective genomic region in the third plurality of genomic regions that is differentially methylated in a cancerous tissue relative to a non-cancerous tissue that are significantly unlikely to be derived from a non-cancerous tissue or, conversely, significantly likely to be derived from a cancerous tissue. For instance, as described in Example 2, identification of as few as 150 ‘unlikely’ nucleic acid sequences can be used to differentiate a liquid biopsy sample from a subject with cancer from a liquid biopsy sample from a subject without cancer, independent of the number of nucleic acid sequences analyzed.

In some embodiments, a respective nucleic acid sequence is significantly unlikely to be derived from a non-cancerous tissue or, significantly likely to be derived from a cancerous tissue (hereinafter, referred to as ‘unlikely nucleic acid sequences’), when a probability determined for the nucleic acid sequence is significantly different than a measure of central tendency for the distribution of probabilities for all of the nucleic acid sequences are derived from a cancerous tissue or from a non-cancerous tissue.

In some embodiments, a nucleic acid sequence is an unlikely nucleic acid sequence when the probability determined for whether the nucleic acid sequence was derived from a cancerous cell is at least 20% greater than a measure of central tendency (e.g., a mean) for the distribution of all probabilities determined for the set of nucleic acid sequences. In some embodiments, a nucleic acid sequence is an unlikely nucleic acid sequence when the probability determined for whether the nucleic acid sequence was derived from a cancerous cell is at least 25% greater, at least 30% greater, at least 40% greater, at least 50% greater, at least 60% greater, at least 70% greater, at least 80% greater, at least 90% greater, at least 100% greater, at least 125% greater, at least 150% greater, at least 175% greater, at least 200% greater, or more, than a measure of central tendency (e.g., a mean) for the distribution of all probabilities determined for the set of nucleic acid sequences.

In some embodiments, a nucleic acid sequence is an unlikely nucleic acid sequence when the probability determined for whether the nucleic acid sequence was derived from a cancerous cell is at least 0.15 units (on a probability scale of 0 to 1 units) greater than a measure of central tendency (e.g., a mean) for the distribution of all probabilities determined for the set of nucleic acid sequences. In some embodiments, a nucleic acid sequence is an unlikely nucleic acid sequence when the probability determined for whether the nucleic acid sequence was derived from a cancerous cell is at least 0.2 units, at least 0.25 units, at least 0.3 units, at least 0.35 units, at least 0.4 units (on a probability scale of 0 to 1 units), or greater than a measure of central tendency (e.g., a mean) for the distribution of all probabilities determined for the set of nucleic acid sequences.

In some embodiments, component model 1130 provides an indication that the subject has cancer when at least a threshold number of unlikely nucleic acid sequences are identified in the plurality of nucleic acids. In some embodiments, the threshold number of unlikely nucleic acid sequences is at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 170, at least 180, at least 185, at least 190, at least 195, at least 200, at least 205, at least 210, at least 215, at least 220, at least 225, at least 230, at least 235, at least 240, at least 245, at least 250, or more unlikely nucleic acid sequences.

In some embodiments, component model 1130 provides an indication that the subject has cancer when at least one unlikely nucleic acid sequence is detected from a threshold number of regions of the genome.

In some embodiments, component model 1130 determines how many regions of the genome from which an unlikely nucleic acid sequence was identified in at least a subset of the set of mapped nucleic acid sequences. For instance, as described in Example 2, identification of as few as 150 ‘unlikely’ nucleic acid sequences can be used to differentiate a liquid biopsy sample from a subject with cancer from a liquid biopsy sample from a subject without cancer, independent of the number of nucleic acid sequences analyzed.

Accordingly, in some embodiments, component model 1130 provides an indication that the subject has cancer when at least one unlikely nucleic acid sequence is detected from at least a threshold number of differentially methylated regions of the genome. In some embodiments, the threshold number of differentially methylated regions is at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 170, at least 180, at least 185, at least 190, at least 195, at least 200, at least 205, at least 210, at least 215, at least 220, at least 225, at least 230, at least 235, at least 240, at least 245, at least 250, or more differentially methylated regions.

### Component Model 1140—Tissue Deconvolution

In some embodiments, the ensemble model includes a component model trained to generate a component circulating tumor fraction estimate based on a plurality of methylation metrics, e.g., corresponding to component model 1136 as illustrated in FIG. 11. In some embodiments, the plurality of methylation metrics comprises a plurality of CpG-level methylation metrics, where a respective CpG-level methylation metric in the plurality of CpG-level methylation metrics represents a corresponding CpG dinucleotide in a set of CpG dinucleotides in the genome of the species of the subject, and the respective CpG-level methylation metric is determined based on a corresponding fraction of the occurrences of the respective CpG dinucleotide, in the set of mapped nucleic acid sequences, that are methylated, e.g., differentially methylated CpG dinucleotides identified, for example, using one or more feature selection methodologies described above in the section titled “Multi-feature Classifiers and Machine Learning.”

In some embodiments, the set of CpG dinucleotides is at least 100, at least 250, at least 500, at least 1000, at least 2500, at least 5000, at least 10,000, at least 25,000, at least 50,000, at least 100,000, at least 250,000, at least 500,000, at least 1,000,000, at least 2,000,000, or more CpG dinucleotides.

In some embodiments, the component model: (i) deconvolves the proportion of non-cancerous and cancerous tissues represented in the plurality of cell-free DNA fragments using the plurality of CpG-level methylation metrics, and (ii) generates the third component circulating tumor fraction estimate based on the total proportion of cancerous tissues represented in the plurality of cell-free DNA fragments. Methods for deconvolving the proportion of non-cancerous and cancerous tissues are known in the art. For example, using a non-negative least-squares (NNLS) algorithm, as described in Schmidt, M., Maić, T., Dahl, E. et al. Deconvolution of cellular subsets in human tissue based on targeted DNA methylation analysis at individual CpG sites. BMC Biol 18, 178 (2020), which is incorporated herein by reference, in its entirety, for all purposes, and specifically for its teaching of a representative method for deconvoluting tissue types based on methylation patterns. In some embodiments, the component circulating tumor fraction estimate is the proportion of cancerous tissues represented in the plurality of cell-free DNA fragments.

In some embodiments, the ensemble model includes a component model trained to generate a component circulating tumor fraction estimate based on a plurality of methylation metrics, e.g., corresponding to component model 1136 as illustrated in FIG. 11. In some embodiments, the plurality of methylation metrics comprises a plurality of bin-level methylation metrics, where a respective bin-level methylation metric in the plurality of bin-level methylation metrics represents a corresponding region of the genome of the species of the subject in a plurality of regions of the genome of the species of the subject, and the respective bin-level methylation metric is determined based on one or more methylation patterns of nucleic acid sequences, in the set of mapped nucleic acid sequences, that map to the respective genomic region.

In some embodiments, the plurality of regions of the genome of the species of the subject is at least 100, at least 250, at least 500, at least 1000, at least 2500, at least 5000, at least 10,000, at least 25,000, at least 50,000, at least 100,000, at least 250,000, at least 500,000, or more regions of the genome.

In some embodiments, the component model: (i) deconvolves the proportion of non-cancerous and cancerous tissues represented in the plurality of cell-free DNA fragments using the plurality of bin-level methylation metrics, and (ii) generates the third component circulating tumor fraction estimate based on the total proportion of cancerous tissues represented in the plurality of cell-free DNA fragments. Methods for deconvolving the proportion of non-cancerous and cancerous tissues are known in the art. For example, using a non-negative least-squares (NNLS) algorithm, as described in Schmidt, M., Maié, T., Dahl, E. et al. Deconvolution of cellular subsets in human tissue based on targeted DNA methylation analysis at individual CpG sites. BMC Biol 18, 178 (2020), which is incorporated herein by reference, in its entirety, for all purposes, and specifically for its teaching of a representative method for deconvoluting tissue types based on methylation patterns. In some embodiments, the component circulating tumor fraction estimate is the proportion of cancerous tissues represented in the plurality of cell-free DNA fragments.

### Component Model 1124—Bin-Level Fragment Length

In some embodiments, the ensemble model includes a component model trained to generate a component circulating tumor fraction estimate based on a plurality of fragment length metrics, e.g., corresponding to component model 1124 as illustrated in FIG. 11. In some embodiments, the plurality of fragment length metrics include a plurality of bin-level fragment size metrics, where each respective bin-level fragment size metric in the plurality of bin-level fragment size metrics represents a corresponding genomic region in a fourth plurality of genomic regions. In some embodiments, the fourth plurality of genomic regions is at least 25, at least 50, at least 100, at least 250, at least 500, at least 1000, at least 2500, at least 5000, at least 10,000, at least 25,000, at least 50,000, at least 100,000, at least 250,000, or more genomic regions.

Non-limiting examples of the types of component models, and they ways in which the component models can be combined in the ensemble model, that can be used in the methods and systems provided herein are described herein, for example, in the sections titled “Multi-feature Classifiers and Machine Learning,” and “Ensemble Models.” In some embodiments, the first component model is a probabilistic model, a deep learning model, or an admixture model.

In some embodiments, a respective bin-level fragment size metric is determined based on a comparison of (i) the abundance of nucleic acid sequences, in the set of mapped nucleic acid sequences that map to the corresponding genomic region, having a length that satisfies a minimal length threshold, to (ii) the abundance of nucleic acid sequences, in the set of mapped nucleic acid sequences that map to the corresponding genomic region, having a length that does not satisfy the minimal length threshold.

In some embodiments, a bin-level fragment size metric corresponds to a measure of central tendency for a distribution of probabilities that respective nucleic acid fragments, corresponding to respective nucleic acid sequence in the plurality of nucleic acid sequences that map to a respective genomic region, are derived from a cancerous tissue and/or is derived from a non-cancerous tissue.

In some embodiments, a bin-level fragment size metric corresponds to a distribution of fragments lengths for the respective nucleic acid fragments, corresponding to respective nucleic acid sequence in the plurality of nucleic acid sequences that map to a respective genomic region. In some embodiments, such fragment length distributions are input into a admixture model in order to determine an estimate as to what proportion of nucleic acid fragments are likely members of a first distribution of nucleic acid fragments derived from a cancerous tissue and what proportion of nucleic acid fragments are likely members of a second distribution of nucleic acid fragments that are derived from a non-cancerous tissue.

### Component Model 1120—Fragment-Level Fragment Length

In some embodiments, the ensemble model includes a component model trained to generate a component circulating tumor fraction estimate based on a plurality of fragment length metrics, e.g., corresponding to component model 1120 as illustrated in FIG. 11. In some embodiments, the plurality of fragment length metrics include a plurality of bin-level fragment size metrics, where each respective fragment-level fragment size metric in the plurality of fragment-level fragment size metrics represents a respective nucleic acid sequence, in at least a subset of the set of mapped nucleic acid sequences, and the respective fragment-level fragment size metric is based on the length of the DNA fragment corresponding to the respective nucleic acid sequence.

Non-limiting examples of the types of component models, and they ways in which the component models can be combined in the ensemble model, that can be used in the methods and systems provided herein are described herein, for example, in the sections titled “Multi-feature Classifiers and Machine Learning,” and “Ensemble Models.” In some embodiments, the first component model is a probabilistic model, a deep learning model, or an admixture model.

In some embodiments, the component model estimates the fraction of the plurality of cell-free DNA fragments that originated from cancerous tissue by fitting the plurality of fragment-level fragment size metrics against (i) one or more normal reference distributions for the length of cell-free DNA originating from non-cancerous tissue, and (ii) one or more cancer reference distributions for the length of cell-free DNA originating from cancerous tissue.

In some embodiments, the one or more normal reference distributions for the length of cell-free DNA originating from non-cancerous tissue comprises a plurality of normal reference distributions, wherein each respective normal reference distribution in the plurality of normal reference distributions is for a distribution of DNA fragment lengths for cell-free DNA fragments originating from non-cancerous tissue that map to a respective genomic region in a fifth plurality of genomic regions. In some embodiments, the one or more cancer reference distributions for the length of cell-free DNA originating from cancerous tissue comprises a plurality of cancer reference distributions, wherein each respective cancer reference distribution in the plurality of cancer reference distributions is for a distribution of DNA fragment lengths for cell-free DNA fragments originating from cancerous tissue that map to a respective genomic region in the fifth plurality of genomic regions.

In some embodiments, the fifth plurality of genomic regions is at least 50, at least 100, at least 250, at least 500, at least 1000, at least 2500, at least 5000, at least 10,000, at least 25,000, at least 50,000, at least 100,000, at least 250,000, or more genomic regions

In some embodiments, the fitting is an iterative process comprising modeling an expected distribution of fragment lengths at each of a plurality of simulated circulating tumor fractions and identifying the model that best fits the plurality of fragment-level fragment size metrics.

### Component Model 1114—Copy Number Variation

In some embodiments, the ensemble model includes a component model trained to generate a component circulating tumor fraction estimate based on a plurality of copy number metrics, e.g., corresponding to component model 1114 as illustrated in FIG. 11. An example method for estimating circulating tumor fraction from copy number variations is ichorCNA, as described in Adalsteinsson, Viktor A et al. “Scalable whole-exome sequencing of cell-free DNA reveals high concordance with metastatic tumors,” Nature communications, 8(1):1324 (2017), which is incorporated by reference herein, in its entirety, for all purposes.

In some embodiments, the analysis of aligned sequence reads, e.g., in SAM or BAM format, includes determination of the copy number 135 for one or more locus, using a copy number variation analysis module 153. In some embodiments, where both a liquid biopsy sample and a normal tissue sample of the patient are analyzed, de-duplicated BAM files and a VCF generated from the variant calling pipeline are used to compute read depth and variation in heterozygous germline SNVs between sequencing reads for each sample. By contrast, in some embodiments, where only a liquid biopsy sample is being analyzed, comparison between a tumor sample and a pool of process-matched normal controls is used. In some embodiments, copy number analysis includes application of a circular binary segmentation algorithm and selection of segments with highly differential log 2 ratios between the cancer sample and its comparator (e.g., a matched normal or normal pool). In some embodiments, approximate integer copy number is assessed from a combination of differential coverage in segmented regions and an estimate of stromal admixture (for example, tumor purity, or the portion of a sample that is cancerous vs. non-cancerous, such as a tumor fraction for a liquid biopsy sample) is generated by analysis of heterozygous germline SNVs.

For instance, in an example implementation, copy number variants (CNVs) are analyzed using the CNVkit package. Talevich et al., PLOS Comput Biol, 12:1004873 (2016), the content of which is hereby incorporated by reference, in its entirety, for all purposes. CNVkit is used for genomic region binning, coverage calculation, bias correction, normalization to a reference pool, segmentation and visualization. The log 2 ratios between the tumor sample and a pool of process matched healthy samples from the CNVkit output are then annotated and filtered using statistical models whereby the amplification status (amplified or not-amplified) of each gene is predicted and non-focal amplifications are removed.

### Ensemble Models

Meta-learning or ensemble learning is an artificial intelligence algorithm development strategy that combines multiple classes of algorithms in an efficient way of performing a classification task. See, for example, Zhou, 2012, “Ensemble Methods: Foundations and Algorithms,” Chapman Hall; Vilalta and Drissi, “A Perspective View and Survey of Meta-Learning,” Artificial Intelligence Review 18(2): 77-95; Chan and Stolfo, 1995, “A comparative evaluation of voting and meta-learning on partitioned data,” paper presented at ICML 1995; and Seewald and Fürnkranz, “An Evaluation of Grading Classifiers,” in Hoffmann et al., Advances in Intelligent Data Analysis: 4th International Conference, IDA 2001 Cascais, Portugal, Sep. 13-15, 2001 Proc. Springer Berlin Heidelberg; 2001:115-124, each of which is hereby incorporated herein by reference in its entirety.

Binary prediction learning algorithms comprising singleton algorithms using relatively small numbers (e.g., 100 cases per group or less) are prone to overfitting. See Rokach, 2010, Pattern Classification Using Ensemble Methods, World Scientific Publishing Co., Inc.; and Frey et al., 2014, “Big Data Deep Phenotyping: Contribution of the IMIA Genomic Medicine Working Group,” Yearbook of Medical Informatics 9(1):206-211, each of which is hereby incorporated herein by reference in its entirety. There are multiple ways to improve these situations including improvement of single algorithms. These include bagging, boosting, or both. In some embodiments, classifiers are improved by using multiple independent learners, evaluating the results of each learner based on concordance estimations, running the prediction task and gathering final results based on approximations from individual learners. See, for example, Breiman, “Bagging predictors,” Machine Learning 24(2):123-140; Freund, 1995, “Boosting a weak learning algorithm by majority,” Inf. Comput. 121(2):256-285; Alceu et al., 2014 “Dynamic selection of classifiers-A comprehensive review,” Pattern Recogn. 47 (11): 3665-3680; and Micha et al., 2014 “A survey of multiple classifier systems as hybrid systems, Inf. Fusion 16:3-17, each of which is hereby incorporated herein by reference in its entirety.

In some embodiments, the ensemble model used in the methods and systems described herein is a bootstrap aggregating ensemble. Bootstrap aggregating is a form of ensemble modeling in which the output of each component model in the ensemble is given an evenly weighted vote in the final output of the ensemble model.

In some embodiments, an ensemble model used in the methods and systems described herein generates a plurality of component estimates of circulating tumor fraction, using one or more of the copy number variation, fragment length, and methylation pattern features derived from a whole genome methylation sequencing reaction as described herein, and generates a final circulating tumor fraction estimate for the sample by taking a measure of central tendency for some or all of the component estimates.

In some embodiments, an ensemble model used in the methods and systems described herein generates a plurality of component estimates of circulating tumor fraction, using one or more of the copy number variation, fragment length, and methylation pattern features derived from a whole genome methylation sequencing reaction as described herein, and generates a final circulating tumor fraction estimate for the sample by taking a weighted measure of central tendency for some or all of the component estimates. Generally, the weights used in the ensemble model are learned during training of the ensemble model. In some embodiments, the weights applied to the component estimates are dependent upon the range of circulating tumor fraction the sample appears to fall into. That is, in some embodiments, different component models are emphasized more than other component models when the circulating tumor fraction of the sample is relatively low (e.g., below a threshold that is no more than 0.25, no more than 0.2, no more than 0.15, no more than 0.1, no more than 0.05, no more than 0.025, no more than 0.01, or less) and/or relatively high (e.g., above a threshold of no less than 0.2, no less than 0.25, no less than 0.3, no less than 0.4, no less than 0.5, or greater).

In some embodiments, the ensemble model used in the methods and systems described herein is a Bayesian model averaging ensemble model. This model generates a weighted measure of central tendency (e.g., an average) for some or all of the component estimates, where the weights are determined by the posterior probability of each model given the data. For additional information on Bayesian model averaging see, for example, Hocting J A, et al., “Bayesian model averaging: a tutorial,” Statist. Sci. 14(4): 382-417 (November 1999), which is incorporated herein by reference, in its entirety, for all purposes.

In some embodiments, the ensemble model used in the methods and systems described herein is a Bayesian model combination ensemble model. This model is an algorithmic correction to Bayesian model averaging, which samples from the space of possible ensembles. For additional information on Bayesian model combination see, for example, Monteith K, et al., “Turning Bayesian Model Averaging into Bayesian Model Combination,” Proceedings of the International Joint Conference on Neural Networks IJCNN'11, 2657-63 (2011), which is incorporated herein by reference, in its entirety, for all purposes.

In some embodiments, the ensemble model used to estimate circulating tumor fraction is a Bayes optimal classifier. The Bayes optimal classifier is a probabilistic model that makes the most probable prediction for a new example, given the training dataset. Specifically, in a Bayes optimal classifier, the conditional probability for a new instance (vi) given the training data (D), given a space of hypotheses (H) is calculated according to P(vⱼ|D)=sum {h in H} P(vⱼ|hᵢ)*P(hᵢ|D), where vⱼ is a new instance to be classified, H is the set of hypotheses for classifying the instance, hᵢ is a given hypothesis, P(vⱼ|hᵢ) is the posterior probability for vⱼ given hypothesis hᵢ, and P(hᵢ|D) is the posterior probability of the hypothesis hᵢ given the data D. Selecting the outcome with the maximum probability is an example of a Bayes optimal classification, e.g., the max sum {h in H} P(vⱼ|hᵢ)*P(hᵢ|D).

In some embodiments, an ensemble model used herein includes one or more chains of component models (e.g., models or learners), where the output of a first component model is used as an input in a second classifier in the downstream classification cascade.

In some embodiments, the ensemble model used to estimate circulating tumor fraction is a stacking model. In such an ensemble, a plurality of component estimates of circulating tumor fraction are generated, using one or more of the copy number variation, fragment length, and methylation pattern features derived from a whole genome methylation sequencing reaction as described herein, and then used as inputs into a trained learning algorithm, e.g., a logistic regression algorithm, a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, a linear regression algorithm, etc.

By using a combination of classifiers, a relatively small patient population can be used to produce a trained ensemble classifier that has a high degree of accuracy. This is advantageous because large training populations can be difficult to obtain, such as when sample acquisition involves invasive procedures, limited patient access, and/or rare or precious sample specimens.

Thus, in some embodiments, an ensemble learning strategy (e.g., an ensemble model) is employed for estimating a circulating tumor fraction for a liquid biopsy sample of a test subject. In some embodiments, the ensemble model comprises a majority voting method and/or a concordance method. In some embodiments, the ensemble model further comprises a k-fold cross validation approach to assessing sample-induced bias and error rates.

In some embodiments, one or more component models in the ensemble model is implemented as an artificial intelligence engine and may include gradient boosting models, random forest models, neural networks (NN), regression models, Naive Bayes models, and/or machine learning algorithms (MLA). A MLA or a NN may be trained from a training data set that includes one or more features derived from whole genome methylation sequencing, e.g., copy number states, methylation patterns, and/or fragment lengths. MLAs include supervised algorithms (such as algorithms where the features/classifications in the data set are annotated) using linear regression, logistic regression, decision trees, classification and regression trees, naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

While MLA and neural networks identify distinct approaches to machine learning, the terms may be used interchangeably herein. Thus, a mention of MLA may include a corresponding NN or a mention of NN may include a corresponding MLA unless explicitly stated otherwise. Training may include providing optimized datasets, labeling these traits as they occur in patient records, and training the MLA to predict or classify based on new inputs. Artificial NNs are efficient computing models which have shown their strengths in solving hard problems in artificial intelligence. They have also been shown to be universal approximators, that is, they can represent a wide variety of functions when given appropriate parameters.

In some embodiments, system 100 includes a classifier training module that includes instructions for training one or more untrained or partially trained classifiers based on feature data from a training dataset. In some embodiments, system 100 also includes a database of training data for use in training the one or more classifiers. In other embodiments, the classifier training module accesses a remote storage device hosting training data.

In some embodiments of the methods and systems described herein, a circulating tumor fraction estimate generated by a component model and/or an ensemble model as described herein is informed by the results of a previous analysis of sequencing data from a cancerous tissue of a subject. In some embodiments, the previous analysis includes sequence analysis of a solid tumor sample. In some embodiments, the previous analysis includes analysis of a liquid biopsy sample from the subject. In some embodiments, the identity of one or more genomic variations identified in the previous sequence analysis informs the circulating tumor fraction estimate. For instance, where it was previously identified that a cancerous tissue of the subject carried a particular genomic mutation, the proportion of sequences encompassing the loci of the genomic mutation that include the genomic mutation in a subsequent sequencing reaction can inform what proportion of all sequenced nucleic acids are derived from the cancerous tissue.

In some embodiments, a previous sequencing analysis, e.g., of a sample of a solid tumor sample from a subject, results in classification of a cancer characteristic, e.g., a type of cancer, a subtype of cancer, an HRD status, a mutational load of the cancer, etc. In some embodiments, the previous classification of the cancer informs a tumor fraction estimate generated by a component model and/or an ensemble model as described herein. In some embodiments, the previous classification of the cancer informs selection of a particular component and/or ensemble model for estimating circulating tumor fraction. For example, in some embodiments, component models for estimating circulating tumor fraction analyze metrics for different differentially methylated regions (e.g., different bins) depending on the type and/or subtype of cancer being evaluated. In some embodiments, this is because different regions of the genome are differentially methylated in different cancers.

In some embodiments, the methods described herein include a step of generating a clinical report 139-3 (e.g., a patient report) providing clinical support for personalized cancer therapy, using the information curated from sequencing of a liquid biopsy sample, as described above. In some embodiments, the report is provided to a patient, physician, medical personnel, or researcher in a digital copy (for example, a JSON object, a pdf file, or an image on a website or portal), a hard copy (for example, printed on paper or another tangible medium). A report object, such as a JSON object, can be used for further processing and/or display. For example, information from the report object can be used to prepare a clinical laboratory report for return to an ordering physician. In some embodiments, the report is presented as text, as audio (for example, recorded or streaming), as images, or in another format and/or any combination thereof.

The report includes information related to the specific characteristics of the patient's cancer, e.g., detected genetic variants, epigenetic abnormalities, associated oncogenic pathogenic infections, and/or pathology abnormalities. In some embodiments, other characteristics of a patient's sample and/or clinical records are also included in the report. For example, in some embodiments, the clinical report includes information on clinical variants, e.g., one or more of copy number variants (e.g., for actionable genes CCNE1, CD274 (PD-L1), EGFR, ERBB2 (HER2), MET, MYC, BRCA1, and/or BRCA2), fusions, translocations, and/or rearrangements (e.g., in actionable genes ALK, ROS1, RET, NTRK1, FGFR2, FGFR3, NTRK2 and/or NTRK3), pathogenic single nucleotide polymorphisms, insertion-deletions (e.g., somatic/tumor and/or germline/normal), therapy biomarkers, microsatellite instability status, and/or tumor mutational burden.

### Variant Characterization

In some embodiments, a predicted functional effect and/or clinical interpretation for one or more identified variants is curated by using information from variant databases. In some embodiments, a weighted-heuristic model is used to characterize each variant.

In some embodiments, identified clinical variants are labeled as “potentially actionable”, “biologically relevant”, “variants of unknown significance (VUSs)”, or “benign”. Potentially actionable alterations are protein-altering variants with an associated therapy based on evidence from the medical literature. Biologically relevant alterations are protein-altering variants that may have functional significance or have been observed in the medical literature but are not associated with a specific therapy. Variants of unknown significance (VUSs) are protein-altering variants exhibiting an unclear effect on function and/or without sufficient evidence to determine their pathogenicity. In some embodiments, benign variants are not reported. In some embodiments, variants are identified through aligning the patient's DNA sequence to the human genome reference sequence version hg19 (GRCh37). In some embodiments, actionable and biologically relevant somatic variants are provided in a clinical summary during report generation.

For instance, in some embodiments, variant classification and reporting is performed, where detected variants are investigated following criteria from known evolutionary models, functional data, clinical data, literature, and other research endeavors, including tumor organoid experiments. In some embodiments, variants are prioritized and classified based on known gene-disease relationships, hotspot regions within genes, internal and external somatic databases, primary literature, and other features of somatic drivers. Variants can be added to a patient (or sample, for example, organoid sample) report based on recommendations from the AMP/ASCO/CAP guidelines. Additional guidelines may be followed. Briefly, pathogenic variants with therapeutic, diagnostic, or prognostic significance may be prioritized in the report. Non-actionable pathogenic variants may be included as biologically relevant, followed by variants of uncertain significance. Translocations may be reported based on features of known gene fusions, relevant breakpoints, and biological relevance. Evidence may be curated from public and private databases or research and presented as 1) consensus guidelines 2) clinical research, or 3) case studies, with a link to the supporting literature. Germline alterations may be reported as secondary findings in a subset of genes for consenting patients. These may include genes recommended by the ACMG and additional genes associated with cancer predisposition or drug resistance.

In some embodiments, a clinical report 139-3 includes information about clinical trials for which the patient is eligible, therapies that are specific to the patient's cancer, and/or possible therapeutic adverse effects associated with the specific characteristics of the patient's cancer, e.g., the patient's genetic variations, epigenetic abnormalities, associated oncogenic pathogenic infections, and/or pathology abnormalities, or other characteristics of the patient's sample and/or clinical records. For example, in some embodiments, the clinical report includes such patient information and analysis metrics, including cancer type and/or diagnosis, variant allele fraction, patient demographic and/or institution, matched therapies (e.g., FDA approved and/or investigational), matched clinical trials, variants of unknown significance (VUS), genes with low coverage, panel information, specimen information, details on reported variants, patient clinical history, status and/or availability of previous test results, and/or version of bioinformatics pipeline.

In some embodiments, the results included in the report, and/or any additional results (for example, from the bioinformatics pipeline), are used to query a database of clinical data, for example, to determine whether there is a trend showing that a particular therapy was effective or ineffective in treating (e.g., slowing or halting cancer progression), and/or adverse effects of such treatments in other patients having the same or similar characteristics.

In some embodiments, the results are used to design cell-based studies of the patient's biology, e.g., tumor organoid experiments. For example, an organoid may be genetically engineered to have the same characteristics as the specimen and may be observed after exposure to a therapy to determine whether the therapy can reduce the growth rate of the organoid, and thus may be likely to reduce the growth rate of cancer in the patient associated with the specimen. Similarly, in some embodiments, the results are used to direct studies on tumor organoids derived directly from the patient. An example of such experimentation is described in U.S. Provisional Patent Application No. 62/944,292, filed Dec. 5, 2019, the content of which is hereby incorporated by reference, in its entirety, for all purposes.

As illustrated in FIG. 2A, in some embodiments, a clinical report is checked for final validation, review, and sign-off by a medical practitioner (e.g., a pathologist). The clinical report is then sent for action (e.g., for precision oncology applications).

### Digital and Laboratory Health Care Platform:

In some embodiments, the methods and systems described herein are utilized in combination with, or as part of, a digital and laboratory health care platform that is generally targeted to medical care and research. It should be understood that many uses of the methods and systems described above, in combination with such a platform, are possible. One example of such a platform is described in U.S. patent application Ser. No. 16/657,804, filed Oct. 18, 2019, which is hereby incorporated herein by reference in its entirety for all purposes.

For example, an implementation of one or more embodiments of the methods and systems as described above may include microservices constituting a digital and laboratory health care platform supporting analysis of liquid biopsy samples to provide clinical support for personalized cancer therapy. Embodiments may include a single microservice for executing and delivering analysis of liquid biopsy samples to clinical support for personalized cancer therapy or may include a plurality of microservices each having a particular role, which together implement one or more of the embodiments above. In one example, a first microservice may execute sequence analysis in order to deliver genomic features to a second microservice for curating clinical support for personalized cancer therapy based on the identified features. Similarly, the second microservice may execute therapeutic analysis of the curated clinical support to deliver recommended therapeutic modalities, according to various embodiments described herein.

Where embodiments above are executed in one or more micro-services with or as part of a digital and laboratory health care platform, one or more of such micro-services may be part of an order management system that orchestrates the sequence of events as needed at the appropriate time and in the appropriate order necessary to instantiate embodiments above. A microservices-based order management system is disclosed, for example, in U.S. Prov. Patent Application No. 62/873,693, filed Jul. 12, 2019, which is hereby incorporated herein by reference in its entirety for all purposes.

For example, continuing with the above first and second microservices, an order management system may notify the first microservice that an order for curating clinical support for personalized cancer therapy has been received and is ready for processing. The first microservice may execute and notify the order management system once the delivery of genomic features for the patient is ready for the second microservice. Furthermore, the order management system may identify that execution parameters (prerequisites) for the second microservice are satisfied, including that the first microservice has completed, and notify the second microservice that it may continue processing the order to curate clinical support for personalized cancer therapy, according to various embodiments described herein.

Where the digital and laboratory health care platform further includes a genetic analyzer system, the genetic analyzer system may include targeted panels and/or sequencing probes. An example of a targeted panel is disclosed, for example, in U.S. Prov. Patent Application No. 62/902,950, filed Sep. 19, 2019, which is incorporated herein by reference and in its entirety for all purposes. In one example, targeted panels may enable the delivery of next generation sequencing results for providing clinical support for personalized cancer therapy according to various embodiments described herein. An example of the design of next-generation sequencing probes is disclosed, for example, in U.S. Prov. Patent Application No. 62/924,073, filed Oct. 21, 2019, which is incorporated herein by reference and in its entirety for all purposes.

Where the digital and laboratory health care platform further includes a bioinformatics pipeline, the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting nucleic acid (e.g., cfDNA, DNA and/or RNA) read counts aligned to a reference genome. The methods and systems described above may be utilized, for example, to ingest the cfDNA, DNA and/or RNA read counts and produce genomic features as a result.

When the digital and laboratory health care platform further includes an RNA data normalizer, any RNA read counts may be normalized before processing embodiments as described above. An example of an RNA data normalizer is disclosed, for example, in U.S. patent application Ser. No. 16/581,706, filed Sep. 24, 2019, which is incorporated herein by reference and in its entirety for all purposes.

When the digital and laboratory health care platform further includes a genetic data deconvoluter, any system and method for deconvoluting may be utilized for analyzing genetic data associated with a specimen having two or more biological components to determine the contribution of each component to the genetic data and/or determine what genetic data would be associated with any component of the specimen if it were purified. An example of a genetic data deconvoluter is disclosed, for example, in U.S. patent application Ser. No. 16/732,229 and PCT/US19/69161, filed Dec. 31, 2019, U.S. Prov. Patent Application No. 62/924,054, filed Oct. 21, 2019, and U.S. Prov. Patent Application No. 62/944,995, filed Dec. 6, 2019, each of which is hereby incorporated herein by reference and in its entirety for all purposes.

When the digital and laboratory health care platform further includes an automated RNA expression caller, RNA expression levels may be adjusted to be expressed as a value relative to a reference expression level, which is often done in order to prepare multiple RNA expression data sets for analysis to avoid artifacts caused when the data sets have differences because they have not been generated by using the same methods, equipment, and/or reagents. An example of an automated RNA expression caller is disclosed, for example, in U.S. Prov. Patent Application No. 62/943,712, filed Dec. 4, 2019, which is incorporated herein by reference and in its entirety for all purposes.

The digital and laboratory health care platform may further include one or more insight engines to deliver information, characteristics, or determinations related to a disease state that may be based on genetic and/or clinical data associated with a patient and/or specimen. Exemplary insight engines may include a tumor of unknown origin engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, and so forth. An example tumor of unknown origin engine is disclosed, for example, in U.S. Prov. Patent Application No. 62/855,750, filed May 31, 2019, which is incorporated herein by reference and in its entirety for all purposes. An example of an HLA LOH engine is disclosed, for example, in U.S. Prov. Patent Application No. 62/889,510, filed Aug. 20, 2019, which is incorporated herein by reference and in its entirety for all purposes. An example of a tumor mutational burden (TMB) engine is disclosed, for example, in U.S. Prov. Patent Application No. 62/804,458, filed Feb. 12, 2019, which is incorporated herein by reference and in its entirety for all purposes. An example of a PD-L1 status engine is disclosed, for example, in U.S. Prov. Patent Application No. 62/854,400, filed May 30, 2019, which is incorporated herein by reference and in its entirety for all purposes. An additional example of a PD-L1 status engine is disclosed, for example, in U.S. Prov. Patent Application No. 62/824,039, filed Mar. 26, 2019, which is incorporated herein by reference and in its entirety for all purposes. An example of a homologous recombination deficiency engine is disclosed, for example, in U.S. Prov. Patent Application No. 62/804,730, filed Feb. 12, 2019, which is incorporated herein by reference and in its entirety for all purposes. An example of a cellular pathway activation report engine is disclosed, for example, in U.S. Prov. Patent Application No. 62/888,163, filed Aug. 16, 2019, which is incorporated herein by reference and in its entirety for all purposes. An example of an immune infiltration engine is disclosed, for example, in U.S. patent application Ser. No. 16/533,676, filed Aug. 6, 2019, which is incorporated herein by reference and in its entirety for all purposes. An additional example of an immune infiltration engine is disclosed, for example, in U.S. Patent Application No. 62/804,509, filed Feb. 12, 2019, which is incorporated herein by reference and in its entirety for all purposes. An example of an MSI engine is disclosed, for example, in U.S. patent application Ser. No. 16/653,868, filed Oct. 15, 2019, which is incorporated herein by reference and in its entirety for all purposes. An additional example of an MSI engine is disclosed, for example, in U.S. Prov. Patent Application No. 62/931,600, filed Nov. 6, 2019, which is incorporated herein by reference and in its entirety for all purposes.

When the digital and laboratory health care platform further includes a report generation engine, the methods and systems described above may be utilized to create a summary report of a patient's genetic profile and the results of one or more insight engines for presentation to a physician. For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ. The report may include therapies and/or clinical trials matched based on a portion or all of the genetic profile or insight engine findings and summaries. For example, the therapies may be matched according to the systems and methods disclosed in U.S. Prov. Patent Application No. 62/804,724, filed Feb. 12, 2019, which is incorporated herein by reference and in its entirety for all purposes. For example, the clinical trials may be matched according to the systems and methods disclosed in U.S. Prov. Patent Application No. 62/855,913, filed May 31, 2019, which is incorporated herein by reference and in its entirety for all purposes.

The report may include a comparison of the results to a database of results from many specimens. An example of methods and systems for comparing results to a database of results are disclosed in U.S. Prov. Patent Application No. 62/786,739, filed Dec. 31, 2018, which is incorporated herein by reference and in its entirety for all purposes. The information may be used, sometimes in conjunction with similar information from additional specimens and/or clinical response information, to discover biomarkers or design a clinical trial.

When the digital and laboratory health care platform further includes application of one or more of the embodiments herein to organoids developed in connection with the platform, the methods and systems may be used to further evaluate genetic sequencing data derived from an organoid to provide information about the extent to which the organoid that was sequenced contained a first cell type, a second cell type, a third cell type, and so forth. For example, the report may provide a genetic profile for each of the cell types in the specimen. The genetic profile may represent genetic sequences present in a given cell type and may include variants, expression levels, information about gene products, or other information that could be derived from genetic analysis of a cell. The report may include therapies matched based on a portion or all of the deconvoluted information. These therapies may be tested on the organoid, derivatives of that organoid, and/or similar organoids to determine an organoid's sensitivity to those therapies. For example, organoids may be cultured and tested according to the systems and methods disclosed in U.S. patent application Ser. No. 16/693,117, filed Nov. 22, 2019; U.S. Prov. Patent Application No. 62/924,621, filed Oct. 22, 2019; and U.S. Prov. Patent Application No. 62/944,292, filed Dec. 5, 2019, each of which is incorporated herein by reference and in its entirety for all purposes.

When the digital and laboratory health care platform further includes application of one or more of the above in combination with or as part of a medical device or a laboratory developed test that is generally targeted to medical care and research, such laboratory developed test or medical device results may be enhanced and personalized through the use of artificial intelligence. An example of laboratory developed tests, especially those that may be enhanced by artificial intelligence, is disclosed, for example, in U.S. Provisional Patent Application No. 62/924,515, filed Oct. 22, 2019, which is incorporated herein by reference and in its entirety for all purposes.

It should be understood that the examples given above are illustrative and do not limit the uses of the systems and methods described herein in combination with a digital and laboratory health care platform.

The results of the bioinformatics pipeline may be provided for report generation 208. Report generation may comprise variant science analysis, including the interpretation of variants (including somatic and germline variants as applicable) for pathogenic and biological significance. The variant science analysis may also estimate microsatellite instability (MSI) or tumor mutational burden. Targeted treatments may be identified based on gene, variant, and cancer type, for further consideration and review by the ordering physician. In some aspects, clinical trials may be identified for which the patient may be eligible, based on mutations, cancer type, and/or clinical history. A validation step may occur, after which the report may be finalized for sign-out and delivery. In some embodiments, a first or second report may include additional data provided through a clinical dataflow 202, such as patient progress notes, pathology reports, imaging reports, and other relevant documents. Such clinical data is ingested, reviewed, and abstracted based on a predefined set of curation rules. The clinical data is then populated into the patient's clinical history timeline for report generation.

Further details on clinical report generation are disclosed in U.S. patent application Ser. No. 16/789,363 (PCT/US20/180002), filed Feb. 12, 2020, which is hereby incorporated herein by reference in its entirety.

### Specific Embodiments of the Disclosure

In some aspects, the systems and methods disclosed herein may be used to support clinical decisions for personalized treatment of cancer. For example, in some embodiments, the methods described herein identify actionable genomic variants and/or genomic states with associated recommended cancer therapies. In some embodiments, the recommended treatment is dependent upon whether or not the subject has a particular actionable variant and/or genomic status. Recommended treatment modalities can be therapeutic drugs and/or assignment to one or more clinical trials. Generally, current treatment guidelines for various cancers are maintained by various organizations, including the National Cancer Institute and Merck & Co., in the Merck Manual.

In some embodiments, the methods described herein further includes assigning therapy and/or administering therapy to the subject based on the identification of an actionable genomic variant and/or genomic state, e.g., based on whether or not the subject's cancer will be responsive to a particular personalized cancer therapy regimen. For example, in some embodiments, when the subject's cancer is classified as having a first actionable variant and/or genomic state, the subject is assigned or administered a first personalized cancer therapy that is associated with the first actionable variant and/or genomic state, and when the subject's cancer is classified as having a second actionable variant and/or genomic state, the subject is assigned or administered a second personalized cancer therapy that is associated with the second actionable variant. Assignment or administration of a therapy or a clinical trial to a subject is thus tailored for treatment of the actionable variants and/or genomic states of the cancer patient.

### EXAMPLES

### Example 1—The Cancer Genome Atlas (TCGA)

The Cancer Genome Atlas (TCGA) is a publicly available dataset comprising more than two petabytes of genomic data for over 11,000 cancer patients, including clinical information about the cancer patients, metadata about the samples (e.g. the weight of a sample portion, etc.) collected from such patients, histopathology slide images from sample portions, and molecular information derived from the samples (e.g. mRNA/miRNA expression, protein expression, copy number, etc.). The TCGA dataset includes data on 33 different cancers: breast (breast ductal carcinoma, bread lobular carcinoma) central nervous system (glioblastoma multiforme, lower grade glioma), endocrine (adrenocortical carcinoma, papillary thyroid carcinoma, paraganglioma & pheochromocytoma), gastrointestinal (cholangiocarcinoma, colorectal adenocarcinoma, esophageal cancer, liver hepatocellular carcinoma, pancreatic ductal adenocarcinoma, and stomach cancer), gynecologic (cervical cancer, ovarian serous cystadenocarcinoma, uterine carcinosarcoma, and uterine corpus endometrial carcinoma), head and neck (head and neck squamous cell carcinoma, uveal melanoma), hematologic (acute myeloid leukemia, Thymoma), skin (cutaneous melanoma), soft tissue (sarcoma), thoracic (lung adenocarcinoma, lung squamous cell carcinoma, and mesothelioma), and urologic (chromophobe renal cell carcinoma, clear cell kidney carcinoma, papillary kidney carcinoma, prostate adenocarcinoma, testicular germ cell cancer, and urothelial bladder carcinoma).

### Example 2—Cancer Detection Based on Identifying Differentially Methylated Fragments

In silico modeling was used to determine whether simple detection of cell-free DNA fragments with methylation patterns that are significantly unlikely to be derived from a non-cancerous tissue could be used to differentiate cell-free samples, e.g., blood, from subjects with cancer and subjects without cancer. Briefly, sequences of cell-free DNA mapping to differentially methylated regions of the human genome, that were generated by whole genome methylation sequencing of (i) blood samples from 5 NSCLC patients with high circulating tumor fractions (a high titer of cfDNA fragments originating from cancerous cells), and (ii) five blood samples from subjects who did not have cancer, were randomly sampled in silico and mixed in silico to form 10 sets (each cancer sample was mixed into two non-cancer samples) of 30,000 sequences at each of tumor titer fractions of 1×10⁻⁷, 0.0001, 0.001, 0.005, 0.01, and 0.1 (open circles). Each non-cancer sample was then sampled and mixed into a different non-cancer sample, to control for the effects of the in silico mixing procedure, creating 30 sets of 30,000 sequences (closed circles).

The differentially methylated regions (DMRs) were identified using training data consisting of methylated sequencing data from healthy plasma, plasma from lung cancer patients, and solid tumor samples from lung cancer (clinical) patients. A region was considered a DMR if DNA fragments from the healthy plasma was either hypermethylated or hypomethylated relative to DNA fragments from the plasma from lung cancer patients and/or solid tumor data for that region. An example of DMR selection is provided in Example 4, below.

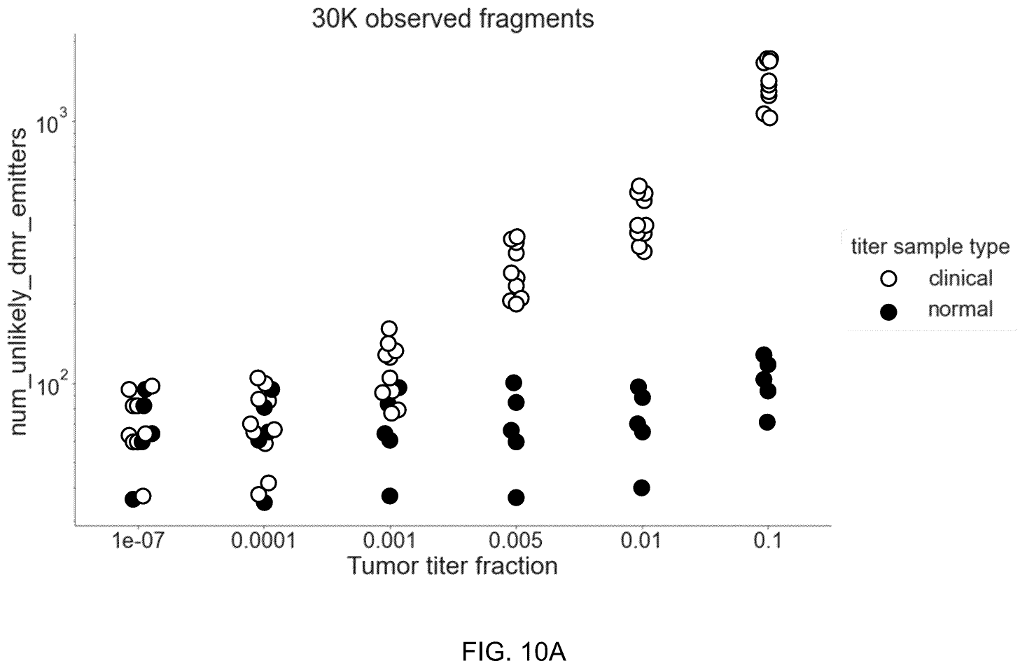

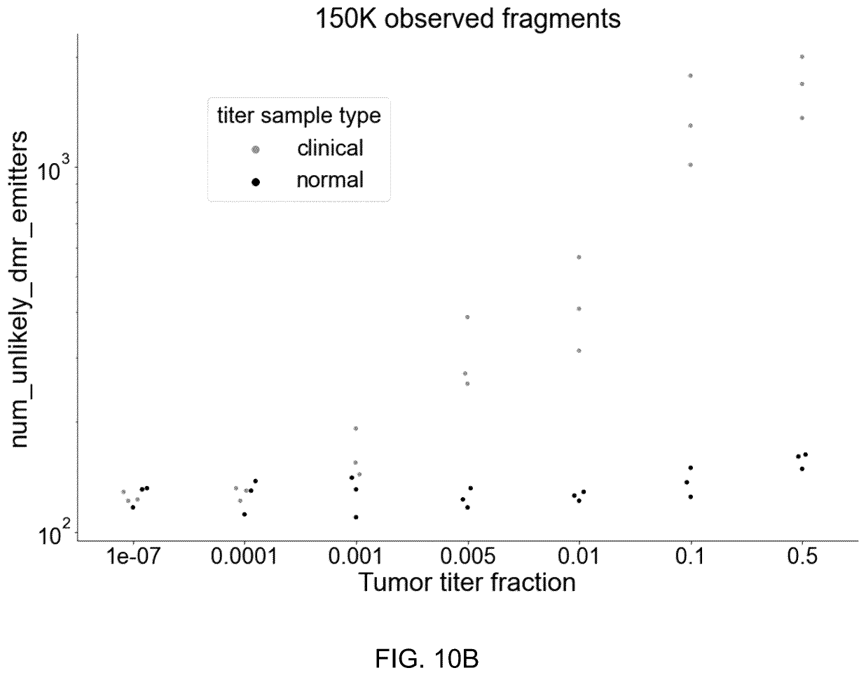

Next, for each sequence in each set of sampled sequences, a probability (from 0 to 1) that the sequence originated from a cancerous cell was calculated by using the kernel density estimation approach described in Example 5. The mean probability for each set of 30,000 sequences (mixed and non-cancerous) was determined and the number of sequences in each set of 30,000 sequences having a probability that was at least 0.3 greater than the mean of the probabilities for the set of 30,000 sequences was determined, referred to as “unlikely sequences.” Next, the number of differentially methylated regions represented by the unlikely sequences for each set of 30,000 sequences was determined and plotted as a function of the in silico tumor fraction for the set of 30,000 sequences, as illustrated in FIG. 10A. As shown in FIG. 10A, separation was achieved between (i) the sets of 30,000 sequences that were mixtures of cancerous and non-cancerous samples, and (ii) the sets of 30,000 sequences sampled exclusively from the non-cancerous sample, at tumor fractions of at least 0.005, corresponding to approximately 150 differentially methylated regions from which at least one unlikely sequence, corresponding to fragments that were highly likely to originate from a cancerous tissue (0.005 TF×30,000 sequences=150 cancerous sequences), were identified.

The in silico experiment was then repeated, but using sets of 150,000 sequences sampled from three NSCLC samples and three non-cancerous samples. As shown in FIG. 10B, separation was achieved between (i) the sets of 150,000 sequences that were mixtures of cancerous and non-cancerous samples, and (ii) the sets of 150,000 sequences sampled exclusively from the non-cancerous sample, at a tumor fraction of at least 0.001, which again corresponded to approximately 150 differentially methylated regions from which at least one unlikely sequence, corresponding to fragments that were highly likely to originate from a cancerous tissue (0.005 TF×30,000 sequences=150 cancerous sequences), were identified. Accordingly, this methodology facilitates cancer identification from methylation sequencing data of a liquid biopsy sample containing unlikely sequences from as few as 150 differentially methylated regions, corresponding to cfDNA fragments that were highly likely to originate from a cancerous tissue.

### Example 3—Comparison of Tumor Fraction Estimates Generated by Analysis of Copy Number Alterations and Bin-Level Methylation Metrics

In order to compare the tumor fraction estimates prepared according to different methodologies, whole genome methylation sequencing was performed on cell-free DNA isolated from blood samples of more than 50 cancer patients and more than 50 patients without cancer. Circulating tumor fractions were then estimated for each sample using either copy number alterations or methylation status of differentially methylated CpG dinucleotides. IchorCNA analysis, as described in Adalsteinsson, Viktor A et al. “Scalable whole-exome sequencing of cell-free DNA reveals high concordance with metastatic tumors,” Nature communications, 8(1):1324 (2017), was used to generate a first tumor fraction estimate for each sample, which is plotted on the x-axis of the graph shown in FIG. 12. In order to generate the second tumor fraction estimate, methylation patterns from differentially methylated regions were evaluated using a probabilistic model that accounts for DNA methylation decay and/or incomplete nucleotide conversion prior to methylation sequencing, as described in Example 5.

As shown in FIG. 12, approximately half of the tumor fraction estimates generated using copy number variation correlated well with their corresponding tumor fraction estimate generated using methylation patterns. These represent the points on the plot, such as point 1212, clustering around line 1210. Significantly, most of the tumor fractions estimates above 0.125 correlated well between the two methodologies. However, many tumor fraction estimate pairs—and particularly for samples where the tumor fraction estimates were below about 0.125—did not correlate well between the two methodologies. This was true both for samples from subjects with cancer and samples from subjects without cancer.

For instance, point 1202 represents a sample from a cancer patient, for which the methylation-based methodology generated a tumor fraction estimate of about 0.125, but the copy number variation-based methodology generated a tumor fraction estimate of about 0. Conversely, point 1204 represents a sample from a cancer patient, for which the methylation-based methodology generated a tumor fraction estimate of about 0, but the copy number variation-based methodology generated a tumor fraction estimate of about 0.2. Thus, both the copy number variation-based methodology and the methylation-based methodology provide false-negative results for samples from cancer patients for which the other methodology correctly identifies a significant tumor fraction.

Both methodologies also provided false positive predictions for the presence of cancer-derived DNA in blood samples of non-cancerous patients. For instance, point 1208 represents a sample from a subject without cancer, for which the methylation-based methodology generated a tumor fraction estimate of about 0, but the copy number variation-based methodology generated a tumor fraction estimate of about 0.125. Similarly, point 1206 represents a sample from a subject without cancer, for which the methylation-based methodology generated a tumor fraction estimate of about 0.3, but the copy number variation-based methodology generated a tumor fraction estimate of about 0. Thus, both the copy number variation-based methodology and the methylation-based methodology provide false-positive results for samples from subject without cancer for which the other methodology correctly identifies a lack of tumor fraction.

### Example 4—Selection of Differentially Methylated Regions (DMRs) by Kernel Density Estimation

A kernel density estimation approach was taken to select a set of differentially methylated regions for use in a component classifier described herein. Candidate regions were identified by either (i) selecting regions identified as differentially methylated in the literature, or (ii) identifying regions containing CpG islands. A training dataset was constructed from whole genome methylation sequencing of samples from a cohort of 92 healthy plasma samples and a cohort of 31 solid tumors from lung cancer patients, e.g., NSCLC patients. For each sample in the cohorts, aligned sequences (corresponding to DNA fragments in the samples) were mapped to the candidate regions. Fragments may only partially overlap. Therefore, for each mapped fragment the number of CpGs that are contained within the candidate region (n) and the number of methylated CpGs that are contained within the candidate region (m) are calculated. Per sample, candidate region, n, and m value a count of fragments was calculated. This is fragment count is normalized to counts per million (CPM) per sample to normalize any coverage differences.

For each candidate region and cohort a bivariate kernel density estimation (KDE) was calculated from the CPM values of all the samples within the cohort, where the independent variables are n and m. To reduce noise, the maximal CPM value from all healthy samples and the minimal CPM value from all solid tumor samples at each point in n,m matrix were used when calculating the KDEs. A bandwidth value of 1.5 was used when estimating the KDE to smooth any noise, however, the bandwidth value can be further optimized. Thus, a KDE represents the empirical likelihood that a fragment with n overlapping CpG sites and m methylated overlapping CpG sites will be found in the respective cohort. This method results in two KDE matrices: KDEₜᵤₘₒᵣ and KDE_{healthy}.

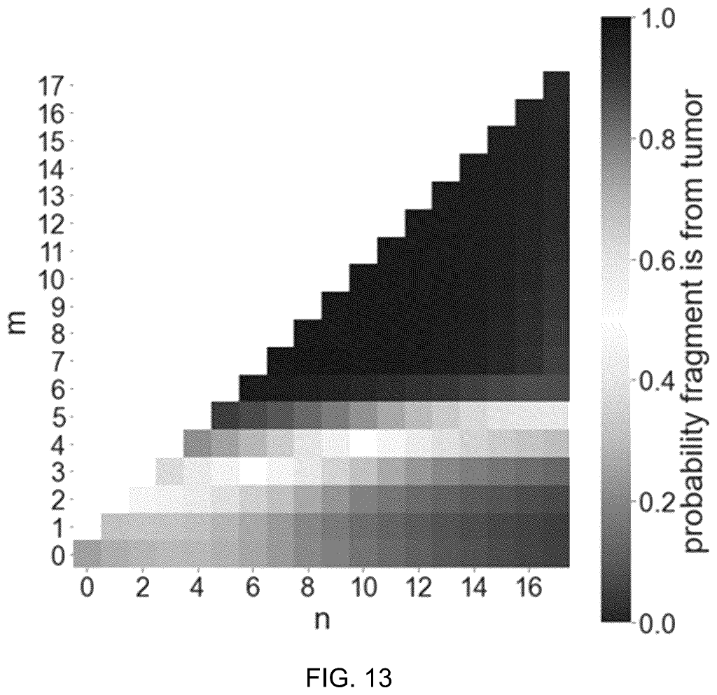

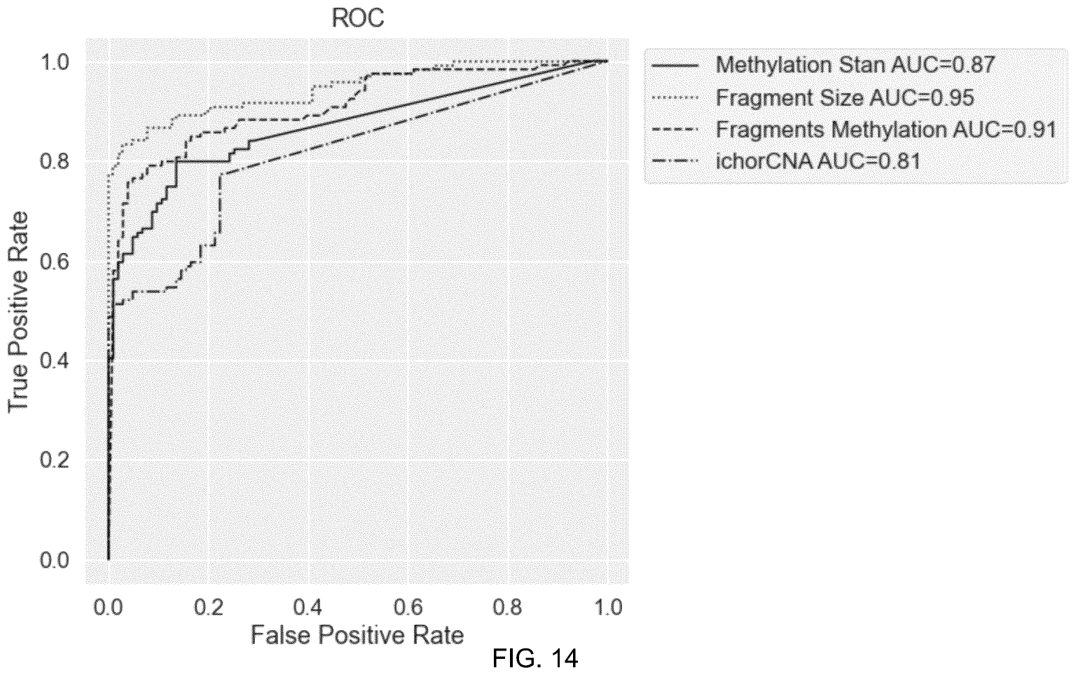

The likelihood values of the KDE were normalized to probabilities as: Normalized Probability=KDEₜᵤₘₒᵣ/(KDEₜᵤₘₒᵣ+KDE_{healthy}). The normalized probability can be interpreted as the “probability a fragment originates from tumor” based on the training data. FIG. 13 shows an example matrix of normalized probabilities. Therefore, in a query sample where a fragment with 14 CpG sites overlapping the candidate region is identified, and >7 of those CpG sites are methylated, there is a high probability that that fragment originated from tumor tissue.

Differentially methylated regions were then selected in two steps. First, DMRs where the median fraction fragment methylation (per fragment defined as m divided by n) in solid tumor samples was 0.2 higher than the median in healthy plasma samples were selected. Then, for each of these regions, the entropy, or Kullback-Liebeler Divergence (KLD) score comparing the KDEₜᵤₘₒᵣ and KDE_{healthy} was calculated. The KLD score is essentially a metric that summarize how different the two distributions are, with higher values representing more divergent distributions. The top 5% or 800 candidate regions with the highest KLD scores were then selected as a set of differentially methylated regions for use in the component models described herein.

### Example 5—Development of Probabilistic Model for Estimating Circulating Tumor Fraction Based on Methylation Patterns of Differentially Methylated Regions

Step 1: Identify features that are informative of DNAm degradation. Select about 100 features, roughly balanced between hypo- and hyper-methylated, that are biologically invariant, e.g., they should have the same methylation level across all tissues present in the samples. These features will help estimate the degree of degradation and batch effects independently of tumor fraction because all observed variability at these features would either come from DNAm degradation (presumably through the loss of methyl-groups) or incomplete enzymatic methyl-conversion. Use these features to estimate the parameters u and vi, the rate of unwanted methylated-to-unmethylated transition and unmethylated-to-methylated transition for sample j, respectively.

Step 2: Identify features that are informative of tumor fraction. Perform an epigenome-wide association analysis (EWAS), meaning that every feature is tested using logistic regression for association with the tumor fraction estimates provided by ichorCNA. The dependent variables are the observed counts of (un)methylated cytosines, the independent variables are the ichorCNA tumor fraction estimates and the parameters u and vi. The tumor fraction estimates are logit-transformed as this will retain the linear relation between dependent and independent variable.

From the results of the EWAS, select about 1000 features to be used as markers to estimate tumor fraction. These markers should
be significantly associated with ichorCNA tumor fraction
explain the observed variability in methylation levels well, assessed by high values for McFadden's R².
be roughly balanced between CpG sites that are hypo/hyper-methylated in tumor.

Step 3: Model definition and assumptions.
μⱼ represents the rate of originally methylated cytosines that are observed as unmethylated, either because of DNAm degradation or incomplete protection by TET2. This rate varies from sample to sample.
vⱼ represents the rate of originally unmethylated cytosines that are observed as methylated as the result of incomplete conversion by APOBEC. This rate varies from sample to sample.
Counts of (un)methylated cytosines, Uᵢⱼ and Mᵢⱼ for feature i and sample j, are generated by a Binomial distribution.
The success probability pᵢⱼ of the Binomial distribution (counting methylated cytosines as successes) depends on several factors.
   For “invariant” features, the success probability depends on the feature-specific methylation level invariant; and the degree of DNAm degradation and methyl-conversion

$${p}_{i j} = {invariant}_{i} \cdot ( 1 - {μ}_{j} ) + ( 1 - {invariant}_{i} ) \cdot {v}_{j}$$

$$P ( {U}_{ij} , {M}_{ij} ) = Binomial ( {U}_{ij} , {M}_{ij} \vert {p}_{i j} )$$

For the other features, the success probability is a mixture of DNA from tumor cells with a methylation level tumor and from normal cells with methylation level normalᵢ and mixture proportions tfᵢ.

$${{p}^{′}}_{i j} = {tumor}_{i} \cdot {tf}_{j} + {normal}_{i} \cdot ( 1 - {tf}_{j} )$$

$${p}_{i j} = {{p}^{′}}_{i j} \cdot ( 1 - {μ}_{j} ) + ( 1 - {{p}^{′}}_{i j} ) \cdot {v}_{j}$$

But a feature may not be informative in all samples. If it is not, it's success probability is qᵢⱼ=normalᵢ·(1−μⱼ)+(1-normalᵢ)·vⱼ.
The observed counts Uᵢⱼ and Mᵢⱼ are therefore generated by a mixture of two Binomial distributions with mixture proportion/weight wᵢ.

$$P ( {U}_{ij} , {M}_{ij} ) = {w}_{i} \cdot Binomial ( {U}_{ij} , {M}_{ij} \vert {p}_{i j} ) + {( 1 - w )}_{i} \cdot Binomial ( {U}_{ij} , {M}_{ij} \vert {q}_{i j} )$$

The data-generating process outlined above is described using the Stan framework. Stan is both a probabilistic programming language and a program that generates a Hamiltonian Monte Carlo sampler in C++ from a model described in Stan. Stan can be used via R or Python bindings.

Step 4: Model training. First, run the Hamiltonian Monte Carlo sampler on the training dataset (counts of (un)methylated cytosines for the features selected in step 1 and 2). Then, start several Markov chains and check for convergence. Finally, extract the mean posterior estimates for the following parameters: normalᵢ, tumorᵢ, wᵢ, invariantᵢ

Step 5: Model predictions for new samples. Use the invariant features selected in step 1 to estimate sample-specific parameters, μⱼ and vⱼ. Once these are determined, the circulating tumor fraction tfⱼ can be numerically estimated by choosing the value that maximizes the likelihood function

$$L = {\prod }_{i} \qquad Binomial ( {U}_{ij} , {M}_{ij} \vert {p}_{i j} )$$

$$with {{p}^{′}}_{i j} = {tumor}_{i} \cdot {tf}_{j} + {normal}_{i} \cdot ( 1 - {tf}_{j} ) and {p}_{i j} = {{p}^{′}}_{i j} \cdot ( 1 - {μ}_{j} ) + ( 1 - {{p}^{′}}_{i j} ) \cdot {v}_{j} .$$

### Example 6—Cancer Detection Using Ensemble Classifier

In order to test whether an ensemble classifier outperforms the various component classifiers described herein, cancer detection models, providing a binary cancer or no cancer assignment, were trained based on bin-level methylation features (Methylation Stan), bin-level fragment size features (Fragment Size), and fragment-level methylation (Fragments Methylation). The bin-level methylation classifier was trained as described in Example 5, to provide circulating tumor fraction estimate (ctFE). To convert this to a binary cancer classification (cancer or no cancer), samples with 95% percentile ctFE value of normal reference samples, within a fold, were classified as cancerous (clinical). The bin-level fragment size classifier was trained as a logistic regression model using 500 bins having 5 MB size (each). The bins were selected by ANOVA based on differential fragment size between cancer and non-cancer samples. The fragment-level classifier was trained as a probabilistic model, similar to that described in Li W., et al., Nucleic Acids Research, 46(15):e89 (2018). Finally, a logistic regression ensemble model incorporating the three component models described above, as well as a classifier based on IchorCNA copy number analysis, was trained using leave one out cross-validation.

Each of the four component models and the ensemble model were then used to classify subjects as having cancer or not, based on whole genome methylation sequencing of cfDNA from blood samples, in a validation study. The cohort included 119 NSCLC cancer patients and 103 non-cancer patients. A ROC curve of the performance of the four component models are presented in FIG. 15. As shown in Table 2, the ensemble model outperformed all of the component models, as evaluated using an accuracy statistic calculated as the sum of the number of true positives divided by the total number of predictions.

TABLE 2
Accuracy of cancer detection models evaluating whole
genome methylation sequencing of cfDNA fragments.

| Model | Accuracy |
|---|---|
| Methylation Stan | 0.801801802 |
| Fragments Methylation | 0.837837838 |
| Fragment Size | 0.896396396 |
| ichorCNA | 0.77027027 |
| ensemble | 0.915857605 |

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1-129. (canceled)

130. A method of monitoring a cancer patient for cancer recurrence, the method comprising:
A) obtaining, in electronic form, a first set of nucleic acid sequences from a methylation sequencing of a plurality of cell-free DNA fragments from a first liquid biopsy sample obtained from the cancer patient, wherein each respective nucleic acid sequence in the first set of nucleic acid sequences comprises a methylation pattern for a corresponding cell-free DNA fragment in the plurality of cell-free DNA fragments;
B) mapping each respective nucleic acid sequence, in the first set of nucleic acid sequences, to a location in a reference construct for the genome of the species of the cancer patient, thereby obtaining a first set of mapped nucleic acid sequences;
C) determining, from the set of mapped nucleic acid sequences, a first set of nucleic acid sequence metrics that is a plurality of methylation metrics for the first liquid biopsy sample, wherein the plurality of methylation metrics comprises a plurality of bin-level methylation metrics, a plurality of fragment-level methylation metrics, or a plurality of CpG-level methylation metrics;
D) making a minimal residual disease (MRD) status prediction for the cancer patient using the plurality of methylation metric;
E) obtaining, in electronic form, a second set of nucleic acid sequences from a sequencing of a plurality of cell-free DNA fragments from a second liquid biopsy sample obtained from the cancer patient;
F) mapping each respective nucleic acid sequence, in the second set of nucleic acid sequences, to a location in a reference construct for the genome of the species of the cancer patient, thereby obtaining a second set of mapped nucleic acid sequences;
G) determining, from the second set of mapped nucleic acid sequences, a second set of nucleic acid sequence metrics that comprises a single nucleotide variation (SNV) or insertion/deletion (INDEL) status at each locus in a plurality of loci; and
H) characterizing a cancer status of the cancer patient using the second set of nucleic acid sequence metrics.

131. The method of claim 130, wherein the methylation sequencing is whole genome methylation sequencing.

132. The method of claim 131, wherein the first set of nucleic acid sequences is at least 25,000 nucleic acid sequences.

133. The method of claim 131, wherein the first set of nucleic acid sequences is at least 250,000 nucleic acid sequences.

134. The method of claim 131, wherein the whole genome sequencing was performed at an average unique sequencing depth of from 0.25 to 3× across the entire genome of the species of the cancer patient.

135. The method of claim 131, wherein the whole genome sequencing was performed at an average unique sequencing depth of from 0.5× to 3× across the entire genome of the species of the cancer patient.

136. The method of claim 131, wherein the whole genome sequencing was performed at an average unique sequencing depth of from 1× to 3× across the entire genome of the species of the cancer patient.

137. The method of claim 131, wherein the whole genome methylation sequencing was performed at an average unique sequencing depth of no more than 3× across the entire genome of the species of the cancer patient.

138. The method of claim 130, wherein the methylation sequencing is targeted-panel methylation sequencing.

139. The method of claim 138, wherein the first set of nucleic acid sequences include sequence reads for 25 or more human genomic loci.

140. The method of claim 138, wherein the first set of nucleic acid sequences include sequence reads for 200 or more human genomic loci.

141. The method of claim 138, wherein the first set of nucleic acid sequences include sequence reads for 50, 100, 150, 250, 300, 350, 400, 500, 750, 1000, 2500, 5000 or more human genomic loci.

142. The method of claim 130, the method further comprises removing one or more germline variants from the plurality of loci prior to characterizing the cancer status.

143. The method of claim 130, wherein the sequencing of the plurality of cell-free DNA fragments from the second liquid biopsy is a targeted-panel sequencing.

144. The method of claim 143, wherein the targeted-panel methylation sequencing makes use of a capture probe for each of at least 10 genes listed in Table 1 thereby enriching the second set of mapped nucleic acid sequences for the at least 10 genes listed in Table 1.

145. The method of claim 143, wherein the targeted-panel methylation sequencing makes use of a capture probe for each of at least 50 genes listed in Table 1 thereby enriching the second set of mapped nucleic acid sequences for the at least 50 genes listed in Table 1.

146. The method of claim 130, wherein the first liquid biopsy sample is blood, whole blood, plasma, serum, urine, cerebrospinal fluid, fecal, saliva, sweat, tears, pleural fluid, pericardial fluid, or peritoneal fluid of the cancer patient.

147. The method of claim 130, wherein the first liquid biopsy sample is a first aliquot and the second liquid biopsy sample is a second aliquot of a common liquid biopsy obtained from the cancer patient.

148. A computer system, comprising one or more processors and memory, the memory storing instructions for performing a method of monitoring a cancer patient for cancer recurrence, the method comprising:
A) obtaining, in electronic form, a first set of nucleic acid sequences from a methylation sequencing of a plurality of cell-free DNA fragments from a first liquid biopsy sample obtained from the cancer patient, wherein each respective nucleic acid sequence in the first set of nucleic acid sequences comprises a methylation pattern for a corresponding cell-free DNA fragment in the plurality of cell-free DNA fragments;
B) mapping each respective nucleic acid sequence, in the first set of nucleic acid sequences, to a location in a reference construct for the genome of the species of the cancer patient, thereby obtaining a first set of mapped nucleic acid sequences;
C) determining, from the set of mapped nucleic acid sequences, a first set of nucleic acid sequence metrics that is a plurality of methylation metrics for the first liquid biopsy sample, wherein the plurality of methylation metrics comprises a plurality of bin-level methylation metrics, a plurality of fragment-level methylation metrics, or a plurality of CpG-level methylation metrics;
D) making a minimal residual disease (MRD) status prediction for the cancer patient using the plurality of methylation metric;
E) obtaining, in electronic form, a second set of nucleic acid sequences from a sequencing of a plurality of cell-free DNA fragments from a second liquid biopsy sample obtained from the cancer patient;
F) mapping each respective nucleic acid sequence, in the second set of nucleic acid sequences, to a location in a reference construct for the genome of the species of the cancer patient, thereby obtaining a second set of mapped nucleic acid sequences;
G) determining, from the second set of mapped nucleic acid sequences, a second set of nucleic acid sequence metrics that comprises a single nucleotide variation (SNV) or insertion/deletion (INDEL) status at each locus in a plurality of loci; and
H) characterizing a cancer status of the cancer patient using the second set of nucleic acid sequence metrics.

149. A non-transitory computer-readable medium storing one or more computer programs, executable by a computer, for making a minimal residual disease (MRD) status prediction for a cancer patient, the computer comprising one or more processors and a memory, the one or more computer programs collectively encoding computer executable instructions that perform a method comprising:
A) obtaining, in electronic form, a first set of nucleic acid sequences from a methylation sequencing of a plurality of cell-free DNA fragments from a first liquid biopsy sample obtained from the cancer patient, wherein each respective nucleic acid sequence in the first set of nucleic acid sequences comprises a methylation pattern for a corresponding cell-free DNA fragment in the plurality of cell-free DNA fragments;
B) mapping each respective nucleic acid sequence, in the first set of nucleic acid sequences, to a location in a reference construct for the genome of the species of the cancer patient, thereby obtaining a first set of mapped nucleic acid sequences;
C) determining, from the set of mapped nucleic acid sequences, a first set of nucleic acid sequence metrics that is a plurality of methylation metrics for the first liquid biopsy sample, wherein the plurality of methylation metrics comprises a plurality of bin-level methylation metrics, a plurality of fragment-level methylation metrics, or a plurality of CpG-level methylation metrics;
D) making a minimal residual disease (MRD) status prediction for the cancer patient using the plurality of methylation metric;
E) obtaining, in electronic form, a second set of nucleic acid sequences from a sequencing of a plurality of cell-free DNA fragments from a second liquid biopsy sample obtained from the cancer patient;
F) mapping each respective nucleic acid sequence, in the second set of nucleic acid sequences, to a location in a reference construct for the genome of the species of the cancer patient, thereby obtaining a second set of mapped nucleic acid sequences;
G) determining, from the second set of mapped nucleic acid sequences, a second set of nucleic acid sequence metrics that comprises a single nucleotide variation (SNV) or insertion/deletion (INDEL) status at each locus in a plurality of loci; and
H) characterizing a cancer status of the cancer patient using the second set of nucleic acid sequence metrics.
